(19) United States
(12) Patent Application Publication
(10) Pub. No.: US 2024/0207270 A1
(43) Pub. Date: Jun. 27, 2024

(54) COMPOUNDS AND COMPOSITIONS FOR MODULATING LIPID METABOLISM

(71) Applicant: WaveBreak Inc., Cambridge, MA (US)

(72) Inventors: Patrick Connelly, Harvard, MA (US); Daniel Kaganovich, Bloomington, IN (US); John A. Thomson, Plymouth, MA (US)

(21) Appl. No.: 18/285,120

(22) PCT Filed: Mar. 30, 2022
(86) PCT No.: PCT/US2022/022631

§ 371 Date: Sep. 29, 2023

Related U.S. Application Data
(60) Provisional application No. 63/168,023, filed on Mar. 30, 2021.

(51) Int. Cl. A61K 31/517; A61K 31/343; A61K 31/40; A61K 31/4458; A61K 31/575; A61K 38/10; G01N 33/50

(52) U.S. Cl. CPC A61K 31/517; A61K 31/343; A61K 31/40; A61K 31/4458; A61K 31/575; A61K 38/10; G01N 33/5008

(57) ABSTRACT

The present disclosure features methods and related compositions that, inter alia, modulate the mitochondrial lipid shunt pathway, including the modulating the cellular mechanisms by which lipids (e.g., fatty acids) are transported between the mitochondria, stress granules, and lipid droplets.

### CLAIM OF PRIORITY

This application claims priority to U.S. Application No. 63/168,023, filed Mar. 30, 2021, the entire contents of which are incorporated herein by reference.

### BACKGROUND

Neurodegenerative diseases are often caused by the malfunctioning of the neuronal and muscular cells in a subject, leading to cell deterioration and death. Currently, over 7 million individuals in the United States alone suffer from a neurodegenerative disease; this number is expected to rise as the average age of the population increases. Current treatment for these conditions is limited, in part due to the lack of understanding of the complex cell biology that underpins the root causes. Indeed, previous efforts to discover curative treatments have been unsuccessful. As such, there is an urgent need for a deeper understanding of the mechanisms of these diseases, as well as new methods and compositions for treatment.

### SUMMARY

The present disclosure features methods and related compositions that, inter alia, modulate lipid metabolism in a cell or subject. In an embodiment, the methods described herein relate to modulation of the mitochondrial lipid shunt pathway, e.g., the cellular mechanisms by which lipids (e.g., fatty acids) are transported between the mitochondria, stress granules, and lipid droplets. In one aspect, the present disclosure features a method of modulating the oxidation of a fatty acid in a cell or subject exhibiting aberrant fatty acid oxidation, comprising administering to the cell or subject a mitochondrial lipid shunt modulator, e.g., a mitochondrial lipid shunt modulator described herein (e.g., a compound of Formulas (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt or ester thereof, e.g., a compound selected from Compound (1)-(573)). In an embodiment, the cell or subject exhibits aberrant fatty acid oxidation prior to administration of the mitochondrial lipid shunt modulator. In an embodiment, the aberrant fatty acid oxidation comprises the prolongation of the elevation of fatty acid oxidation in response to starvation or metabolic stress. In an embodiment, the subject exhibiting aberrant fatty acid oxidation has a neurodegenerative disease, e.g., amyotrophic lateral sclerosis.

Without being bound by theory, the inventors have identified an association (e.g., a physical association) between stress granules, mitochondria, and lipid droplets, and physiological and biochemical evidence of a metabolic control system, whereby stress granules regulate metabolic remodeling in mitochondria and maintain a normal balance of glycolysis, fatty acid oxidation, and other pathways for ongoing energy supply following metabolic stress. Further, the inventors have found that stress granules are capable of down-regulating fatty acid import into mitochondria in normal, heathy cells, thereby limiting dangerous sequelae of excessive fatty acid oxidation during prolonged nutrient stress. The inventors have named this metabolic regulation system a “mitochondrial lipid shunt” and posit that (a) the mitochondrial lipid shunt is an important physiological regulation system in certain tissues, such as neurological and muscular tissues (e.g., neuromuscular junctions), and (b) biochemical failure or disruption of the mitochondrial lipid shunt, by any means, may lead to constitutive or dysregulated fatty acid oxidation upregulation and result in oxidative damage, lipid depletion, and neurodegeneration.

In another aspect, the present disclosure features a method of modulating the formation and/or activity of stress granules in a cell or subject exhibiting aberrant fatty acid oxidation, comprising administering to the cell or subject a mitochondrial lipid shunt modulator. In an embodiment, modulating comprises increasing the formation of (e.g., assisting in the formation of) stress granules (e.g., normal, healthy stress granules). In an embodiment, modulating comprises enhancing formation of stress granules. In an embodiment, increasing stress granule formation results in modulating (e.g., decreasing) VDAC activity or reducing aberrant fatty acid oxidation. In an embodiment, the modulating comprises modulating the association of stress granules with the mitochondria and/or lipid droplets.

In another aspect, the present disclosure features a method of modulating one or more of: (i) the number of stress granules; (ii) morphology of a stress granule, e.g., the size, shape, surface area to volume ratio; (iii) the protein or nucleic acid composition of a stress granule; (iv) the internal mobility or internal dynamics of a stress granule; and (v) the physicochemical phase of a stress granule, in a cell or subject exhibiting aberrant fatty acid oxidation, comprising administering to the cell or subject a mitochondrial lipid shunt modulator, e.g., described herein. In another aspect, the present disclosure features a method of modulating one or more of: (i) the number of lipid droplets; (ii) morphology of a lipid droplet, e.g., the size, shape, surface area to volume ratio; (iii) the protein or nucleic acid composition of a lipid droplet; (iv) the internal mobility or internal dynamics of a lipid droplet; (v) the physicochemical phase of a lipid droplet; and (vi) the agglomeration state of a lipid droplet, in a cell or subject exhibiting aberrant fatty acid oxidation, comprising administering to the cell or subject a mitochondrial lipid shunt modulator, e.g., described herein.

In any embodiment, the mitochondrial lipid shunt modulator comprises a VDAC modulator, a CPT1 modulator, or an ACSL modulator. In some embodiments, the mitochondrial lipid shunt modulator comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt thereof, e.g., as described herein. In some embodiments, the mitochondrial lipid shunt modulator comprises a compound selected from (1)-(573) or a pharmaceutically acceptable salt thereof, e.g., as described herein. In some embodiments, the mitochondrial lipid shunt modulator is selected from AG-17, erastin, erstin, efsevin, olesoxime, teglicar, perhexiline, amiodarone, 2-tetradecylglydate, and PA22, or an analog thereof. In some embodiments, the mitochondrial lipid shunt modulator is selected from AG-17, AG-825, AS-604850, BAY 43-9006, CAY 10561, CAY 10571, D4476, erastin, erstin, efsevin, olesoxime, teglicar, perhexiline, amiodarone, 2-tetradecylglydate, PA22, sulindac, sulindac sulfone (exisulind), itraconazole, torin-1, nintedanib, ruxolitinib, SB-505124, SB-203580, leelamine, LY294002, LY364947, ML-9, O-1918, PD169316, emodin, KN-93, bisindolylmaleimide IX, 17β-hydroxywortmannin, GSK1059615, (S)-H-1152, PD 166326, PI3-Kinase alpha inhibitor 2, piceatannol, pimasertib, RG-13022, roscovitine, SB-202190, SB-415286, SMI-4a, sphingosine inhibitor compound, TGX-221, U0126, and WEHI-9625 or an analog thereof. In an embodiment, the mitochondrial lipid shunt modulator is AG-17. In an embodiment, the mitochondrial lipid shunt modulator is AG-825. In an embodiment, the mitochondrial lipid shunt modulator is AS-604850. In an embodiment, the mitochondrial lipid shunt modulator is BAY 43-9006. In an embodiment, the mitochondrial lipid shunt modulator is CAY 10571. In an embodiment, the mitochondrial lipid shunt modulator is CAY 10561. In an embodiment, the mitochondrial lipid shunt modulator is D4476. In an embodiment, the mitochondrial lipid shunt modulator is erastin. In an embodiment, the mitochondrial lipid shunt modulator is erstin. In an embodiment, the mitochondrial lipid shunt modulator is efsevin. In an embodiment, the mitochondrial lipid shunt modulator is oleosoxime. In an embodiment, the mitochondrial lipid shunt modulator is teglicar. In an embodiment, the mitochondrial lipid shunt modulator is perhexiline. In an embodiment, the mitochondrial lipid shunt modulator is amiodarone. In an embodiment, the mitochondrial lipid shunt modulator is 2-tetradecylglydate. In an embodiment, the mitochondrial lipid shunt modulator is sulindac, sulindac sulfide, or sulindac sulfone (exisulind). In an embodiment, the mitochondrial lipid shunt modulator is itraconazole. In an embodiment, the mitochondrial lipid shunt modulator is torin-1. In an embodiment, the mitochondrial lipid shunt modulator is nintedanib. In an embodiment, the mitochondrial lipid shunt modulator is ruxolitinib. In an embodiment, the mitochondrial lipid shunt modulator is SB-505124. In an embodiment, the mitochondrial lipid shunt modulator is SB-203580. In an embodiment, the mitochondrial lipid shunt modulator is leelamine. In an embodiment, the mitochondrial lipid shunt modulator is LY294002. In an embodiment, the mitochondrial lipid shunt modulator is LY364947. In an embodiment, the mitochondrial lipid shunt modulator is ML-9. In an embodiment, the mitochondrial lipid shunt modulator is O-1918. In an embodiment, the mitochondrial lipid shunt modulator is PD169316. In an embodiment, the mitochondrial lipid shunt modulator is emodin. In an embodiment, the mitochondrial lipid shunt modulator is KN-93. In an embodiment, the mitochondrial lipid shunt modulator is bisindolylmaleimide IX. In an embodiment, the mitochondrial lipid shunt modulator is roscovitine. In an embodiment, the mitochondrial lipid shunt modulator is SB-202190. In an embodiment, the mitochondrial lipid shunt modulator is SB-415286. In an embodiment, the mitochondrial lipid shunt modulator is SMI-4a. In an embodiment, the mitochondrial lipid shunt modulator is sphingosine inhibitor compound. In an embodiment, the mitochondrial lipid shunt modulator is TGX-221. In an embodiment, the mitochondrial lipid shunt modulator is U0126. In an embodiment, the mitochondrial lipid shunt modulator is WEHI-9625. In an embodiment, the mitochondrial lipid shunt modulator is GSK1059615. In an embodiment, the mitochondrial lipid shunt modulator is (S)-H-1152. In an embodiment, the mitochondrial lipid shunt modulator is PD166326. In an embodiment, the mitochondrial lipid shunt modulator is PI3-Kinase alpha inhibitor 2. In an embodiment, the mitochondrial lipid shunt modulator is piceatannol. In an embodiment, the mitochondrial lipid shunt modulator is pimasertib. In an embodiment, the mitochondrial lipid shunt modulator is RG13022. In an embodiment, the mitochondrial lipid shunt modulator is 17p-hydroxywortmannin. In an embodiment, the mitochondrial lipid shunt modulator is PA22. In some embodiments, the mitochondrial lipid shunt modulator modulates the binding of VDAC to GSK3, hexokinase, or tubulin.

In another aspect, the present disclosure features a method of treating a neurodegenerative disease, e.g., amyotrophic lateral sclerosis, in a subject, comprising administering to the subject a mitochondrial lipid shunt modulator described herein. In an embodiment, the subject has or is identified as having a mutation in a gene, e.g., selected from TDP43, FUS, and C9orf72. In an embodiment, the subject has or is identified as having amyotrophic lateral sclerosis. In an embodiment, the subject has amyotrophic lateral sclerosis. In an embodiment, the subject is a mammal, e.g., a human.

The details of one or more embodiments of the disclosure are set forth herein. Other features, objects, and advantages of the disclosure will be apparent from the Detailed Description, the Examples, the Figures, and the Claims.

### BRIEF DESCRIPTIONS OF THE DRAWINGS

FIG. 1 is a schematic overview of the mitochondrial lipid shunt pathway, illustrating exemplary pathway components such as stress granules, lipid droplets, mitochondria, VDAC1, VDAC2, VDAC3, CPT1, ACSL, GSK3, hexokinase or tubulin.

FIGS. 2A-2D are figures that illustrate an exemplary protocol for generating induced pluripotent stem cell (iPSC)-derived neuronal proxy cells with an ALS-like phenotype; each of FIGS. 2A-2D is described in further in the Description and Examples herein.

FIGS. 3A-3C are figures that illustrate an exemplary small molecule screen to identify the correlation between stress granule and lipid droplet formation; each of FIGS. 3A-3C is described in detail in further in the Description and Examples herein.

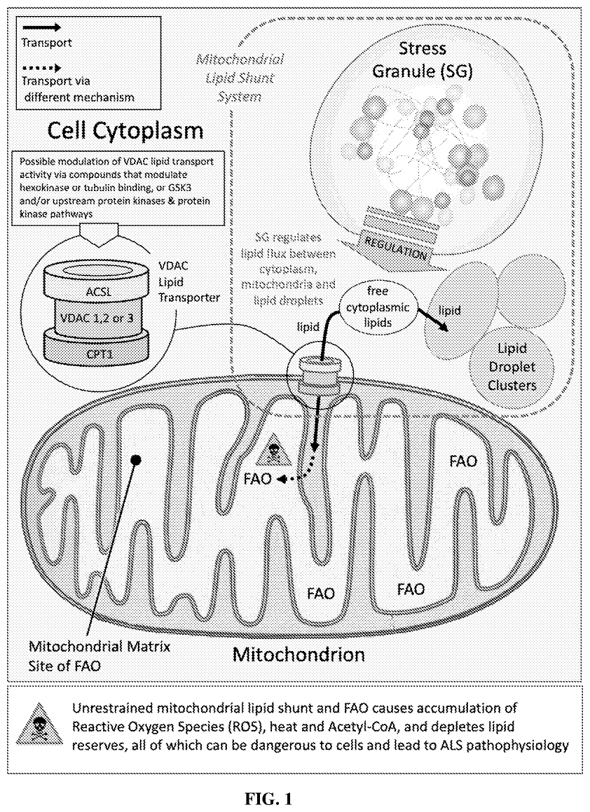

### DETAILED DESCRIPTION

The present disclosure features methods and related compositions for modulating lipid metabolism, as well as related methods of use. In an embodiment, the methods described herein relate to modulation of the mitochondrial lipid shunt pathway, in particular, the interaction between the mitochondria, stress granules, and lipid droplets, by administration of a mitochondrial lipid shunt modulator. In some embodiments, the mitochondrial lipid shunt modulator is a compound described herein (e.g., a compound of Formulas (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt or ester thereof). In some embodiments, the mitochondrial lipid shunt modulator comprises a compound selected from (1)-(573) or a pharmaceutically acceptable salt thereof, e.g., as described herein.

### Definitions

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., microscopy to acquire cellular viability data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a mitochondrial lipid shunt modulator refers to an amount sufficient to elicit the desired biological response, i.e., treating a disease, disorder, or condition. As will be appreciated by those of ordinary skill in this art, an effective amount of a mitochondrial lipid shunt modulator may vary depending on such factors as the desired biological endpoint, the pharmacokinetics of the compound, the condition being treated, the mode of administration, and the age and health of the subject. An effective amount encompasses therapeutic and prophylactic treatment. For example, in treating amyotrophic lateral sclerosis, an effective amount of an inventive compound may decrease the onset of the disease or improve a symptom, e.g., decrease muscle fasciculations.

A “therapeutically effective amount” of a mitochondrial lipid shunt modulator is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a mitochondrial lipid shunt modulator prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a mitochondrial lipid shunt modulator. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₈, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₅alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group.

Exemplary heteroalkyl groups include, but are not limited to: —CH₂—CH₂O—CH₃, —CH₂—CH₂—NH—CH₃, —CH₂—CH₂—N(CH₃)—CH₃, —S—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent. Exemplary heteroaryl groups include pyrrolyl, furanyl and thiophenyl, imidazolyl, pyrazolyl, oxazolyl, isoxazolyl, thiazolyl, isothiazolyl, triazolyl, oxadiazolyl, thiadiazolyl, pyridinyl, pyridazinyl, pyrimidinyl, pyrazinyl, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, and quinazolinyl.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. Exemplary heterocyclyl groups azirdinyl, oxiranyl, thiorenyl. azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dione, dioxolanyl, oxasulfuranyl, disulfuranyl, oxazolidin-2-one, triazolinyl, oxadiazolinyl, and thiadiazolinyl.

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′- and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, “oxo” refers to a carbonyl, i.e., —C(O)—.

The symbol “ [symbol] ” as used herein in relation to a particular mitochondrial lipid shunt modulator (e.g., any one of the Formulas provided herein) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

Compound described herein may also comprise one or more isotopic substitutions. For example, H may be in any isotopic form, including ¹H, ²H (D or deuterium), and ³H (T or tritium); C may be in any isotopic form, including ²C, ³C, and ⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The mitochondrial lipid shunt modulators described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of a electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Mitochondrial Lipid Shunt Pathway

The present disclosure features methods for modulating the mitochondrial lipid shunt pathway. The mitochondrial lipid shunt pathway refers to, inter alia, the complex cellular mechanisms by which cytoplasmic stress granules interact with or otherwise coordinate the activity of mitochondrial outer membrane transport mechanisms to regulate, e.g., the transport of lipids and other vital components of fatty acid beta-oxidation into mitochondria. In this way, cytoplasmic lipids appear to be gated, to either be stored in lipid droplets or transported into mitochondria, where they can be metabolized via beta-oxidation to supply ATP in response to perceived metabolic needs. In some embodiments, genetic and environmental factors can, individually or in combination, disrupt the normal functioning of the mitochondrial lipid shunt pathway. In some embodiments, disruption of the mitochondrial lipid shunt pathway results in a persistent metabolic imbalance. In some embodiments, disruption of the mitochondrial lipid shunt pathway leads to the development of a neurodegenerative disorder, such as amyotrophic lateral sclerosis (ALS). The methods described herein provide for modulation of the mitochondrial lipid shunt pathway and potential treatment of neurodegenerative diseases related to the aberrant functioning thereof, such as ALS.

Exemplary members of the mitochondrial lipid shunt pathway include voltage-dependent anion channel (VDAC) proteins, often termed “pore forming proteins” or “porins.” There are at least 3 human VDACs (VDAC1, VDAC2, VDAC3), which exhibit a high level of genetic and amino acid similarity (Messina et al (Biochim Biophys Acta (2102) 1818, 1466-1476.); Raghavan et al (Biochim Biophys Acta (2012) 1818, 1477-1485.)). Each of VDAC1, VDAC2, and VDAC3 carry out multiple functions in the cell, including electrochemical sensing and trans-membrane transport functions, which can be regulated by complex interactions with other proteins (Naghdi & Hajnoczky (Biochim Biophys Acla (2016) 1863, 2503-2514.); Lee et al (J Biol Chem (2011) 286, 25655-25662.); Sander et al (Int. J. Mol. Sci. (2021) 22, 946. https://doi.org/10.3390/ijms22020946)). Although their tissue-specific expression and possible differential functions and regulation are still incompletely understood, VDAC2 may be more highly expressed and/or play a larger role than the other VDACs in the transport of lipid beta-oxidation raw materials in neuronal cells (Raghavan et al (Biochim Biophys Acta (2012) 1818, 1477-1485.); Naghdi & Hajnoczky (Biochim Biophys Acta (2016) 1863, 2503-2514.)). Although the VDAC proteins appear to be the major central conduit structure for transporting lipids across the mitochondrial outer membrane, they appear to interact in permanent or transient associations with a number of other molecular entities, wherein the entire molecular ensemble, or multi-protein complex, fulfills a common function.

Without being bound by theory, in order for long chain fatty acids to be transported into the mitochondria by VDACs, at least two enzymatic processing steps are usually required. First, the free cytosolic fatty acid may be activated by long chain acyl-CoA synthetase (ACSL), to form the corresponding fatty acyl-CoA adduct. Next, the activated fatty acyl-CoA may be converted to a fatty acylcarnitine adduct by the enzyme carnitine palmitoyltransferase I (CPT1). This latter enzymatic conversion appears to be mechanistically linked to the actual transfer of the fatty acid adduct across the mitochondrial outer membrane by VDACs. This latter linked process comprises the rate-determining step in the overall process of mitochondrial fatty acid beta-oxidation, starting from free cytosolic fatty acids (Lee et al (J Biol Chem (2011) 286, 25655-25662.); Shriver & Manchester (Sci. Rep. (2011) 1, 79. https://doi.org/10.1038/srep00079)). Previous studies provide evidence that ACSL and CPT1 can form a stable, multifunctional complex with any of the known VDACs (VDAC1, VDAC2 and VDAC3), with the multi-protein complex situated in the mitochondrial outer membrane (Lee et al (J Biol Chem (2011) 286, 25655-25662.)). Therefore, the inventors hypothesize that the multi-protein complex acts as a co-regulated ensemble in mitochondrial fatty acid import. As such, it is contemplated that compounds that modulate any of the constituent activities of this ensemble, by binding to VDACs, ACSL or CPT1, may be of pharmacological benefit, for example, by modulating the activity of the mitochondrial lipid shunt pathway. In addition, these mitochondrial lipid shunt modulators may act as effective treatments for subjects having a neurodegenerative disease, such as ALS.

In some embodiments, the methods described herein comprise identification and/or evaluation of a mitochondrial lipid shunt modulator, for modulating the activity of one or more a VDAC, e.g., VDAC2. In an embodiment, modulation of VDAC, e.g., VDAC2, comprises altering the expression of a VDAC (e.g., VDAC2).

Compounds that modulate the mitochondrial lipid shunt pathway may elicit a desired pharmacological outcome by either directly binding to one or more of the VDACs, ACSL or CPT1, or by indirectly interacting with the mitochondrial lipid shunt pathway, for example, by binding to another cellular entity (e.g., a protein kinase or tubulin). As one example, the transport activities of VDACs also appear to be regulated via direct phosphorylation by a number of protein kinases, including Glycogen Synthase Kinase 3 (GSK3) (Sheldon et al (PLoS One (2011) 6, e25539. https://doi.org/10.1371/journal.pone.0025539); Pastorino & Hoek (J. Bioenerg. Biomembr. (2008) 40, 171-182.)). Therefore, a mitochondrial lipid shunt modulator described herein may elicit a desired pharmacological effect by directly binding to and modulating the protein kinase activity of either GSK3 or the upstream protein kinases that, in turn, regulate the activity of GSK3. In another example, binding of hexokinases (HKI & HKII) to VDACs may also affect the lipid transport functions of such VDACs (Pastorino & Hoek (J. Bioenerg. Biomembr. (2008) 40, 171-182.); Dubey et al (Cell Death Discovery (2016) 2, 16085 https://doi.org/10.1038/cddiscovery.2016.85); Ben-Hail et al (J Biol Chem (2016) 291, 24986-25003.); Abu-Hamad et al (J Biol Chem (2008) 283, 13482-13490.)). Moreover, binding of VDACs to hexokinases can also be regulated by GSK3 and other protein kinases (Pastorino & Hoek (J. Bioenerg. Biomembr. (2008) 40, 171-182.)). Therefore, in one embodiment, a mitochondrial lipid shunt modulator described herein may elicit desired pharmacological effects by modulating the binding of hexokinases to VDACs. In another example, binding of free tubulin to VDACs may also affect the lipid transport functions of such VDACs (Naghdi & Hajnoczky (Biochim Biophys Acta (2016) 1863, 2503-2514.); Sheldon et al (PLoS One (2011) 6, e25539; https://doi.org/10.1371/journal.pone.0025539)). As such, in another embodiment, a mitochondrial lipid shunt modulator may elicit desired pharmacological effects by modulating the binding of tubulin to VDACs.

### Mitochondrial Lipid Shunt Modulators

Described herein are mitochondrial lipid shunt modulators as well as related compositions thereof. It is envisioned that a mitochondrial lipid shunt modulator may affect a component in the mitochondrial lipid shunt pathway in the cell. For example, a mitochondrial lipid shunt modulator may modulate the size, number, or morphology of a stress granule or lipid droplet, or modulate the interaction of one or more proteins involved in fatty acid import into mitochondria.

The mitochondrial lipid shunt modulator described herein may comprise a long-chain acyl CoA synthetase (ACSL) modulator, a carnitine palmitoyltransferase I (CPT1) modulator, or a voltage-dependent anion channel (VDAC) modulator. In an embodiment, the mitochondrial lipid shunt modulator comprises an ACSL modulator. In an embodiment, the ACSL modulator results in a decrease in ASCL activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the ACSL modulator results in an increase in ASCL activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard.

In an embodiment, the mitochondrial lipid shunt modulator comprises a CPT1 modulator. In an embodiment, the CPT1 modulator results in a decrease in CPT1 activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the CPT1 modulator results in an increase in CPT1 activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the CPT1 modulator is a L-CPT1 modulator.

In an embodiment, the mitochondrial lipid shunt modulator comprises a VDAC modulator. In an embodiment, the VDAC modulator results in a decrease in VDAC activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the VDAC modulator results in an increase in VDAC activity, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the VDAC modulator is a VDAC1, VDAC2, or a VDAC3 modulator.

In an embodiment, the administration of a mitochondrial lipid shunt modulator to a cell or subject results in an increase in stress granule number and/or size in the cell or subject. In an embodiment, the increase in stress granule number and/or size in the cell or subject is, e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard. In an embodiment, the administration of a mitochondrial lipid shunt modulator to a cell or subject results in an increase in lipid droplet number and/or size in the cell or subject. In an embodiment, the increase in lipid droplet number and/or size in the cell or subject is, e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard.

The mitochondrial lipid shunt modulator described herein may comprise compound that interacts with another member of the mitochondrial lipid shunt pathway, such as glycogen synthase kinase 3 (GSK3), a hexokinase (HK), such as hexokinase I (HKI) or hexokinase II (HKII), or tubulin. In some embodiments, the mitochondrial lipid shunt modulator interacts (e.g., directly or indirectly) with GSK3. In some embodiments, the mitochondrial lipid shunt modulator interacts (e.g., directly or indirectly) with a hexokinase, e.g., HKI or HKII. In some embodiments, the mitochondrial lipid shunt modulator interacts (e.g., directly or indirectly) with tubulin.

In some embodiments, the mitochondrial lipid shunt modulator is a compound disclosed in U.S. Pat. No. 7,879,845 (e.g., a compound of Formula (I) or a salt or derivative thereof), incorporated herein by reference in its entirety. In some embodiments, the mitochondrial lipid shunt modulator is a compound of Formula (I):

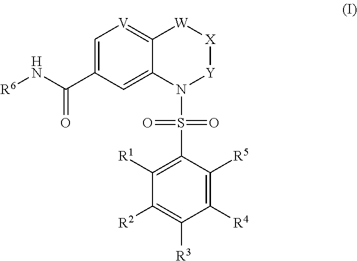

wherein V is C(R⁷)—; W is a single bond; X is S, SO or SO₂; Y is —C(R¹¹R¹²)C(R¹³R¹⁴) or —C(R¹¹)═C(R¹²)—; R¹, R², R³, R⁴ and R⁵ independently from each other are hydrogen, halogen, cyano, hydroxy, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, NH₂—S(O)₂, N(H,lower-alkyl)-S(O)₂ or N(lower-alkyl)₂-S(O)₂, NH₂—C(O), N(H,lower-alkyl)-C(O), N(lower-alkyl)₂-C(O), COOH or lower-alkoxy-C(O), wherein lower-alkyl is optionally substituted with hydroxy, NH₂, N(H,lower-alkyl) or N(lower-alkyl)₂; R⁶ is an aryl or heteroaryl group, which aryl or heteroaryl group is optionally substituted by 1 to 4 substituents selected from the group consisting of halogen, hydroxy, cyano, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, NH₂—S(O)₂, N(H,lower-alkyl)-S(O)₂, N(lower-alkyl)₂-S(O)₂, NH₂—C(O), N(H,lower-alkyl)-C(O), N(lower-alkyl)₂-C(O), lower-alkoxy-C(O), COOH, 1H-tetrazol-5-yl, 5-oxo-4H-[1,2,4]oxadiazol-3-yl, 5-oxo-4H-[1,2,4]thiadiazol-3-yl, 5-thioxo-4H-[1,2,4]oxadiazol-3-yl, 2-oxo-3H-[1,2,3,5]oxathiadiazol-4-yl, SO₃H, 3-hydroxy-isooxazol-5-yl, 6-oxo-6H-pyran-3-yl, 6-oxo-6H-pyran-2-yl, 2-oxo-2H-pyran-3-yl, 2-oxo-2H-pyran-4-yl and P(OXOCH₂CH₃)OH, wherein lower-alkyl is optionally substituted with COOH, hydroxy, NH₂, N(H,lower-alkyl) or N(lower-alkyl)₂, and wherein fluoro-lower-alkyl is optionally substituted with hydroxy; R⁷ is hydrogen, halogen, lower-alkyl, lower-alkoxy, fluoro-lower-alkyl, fluoro-lower-alkoxy, hydroxy or hydroxyl-lower-alkyl; R¹¹, R¹², R¹³ and R¹⁴ independently from each other are hydrogen, halogen, hydroxy, lower alkyl, lower-alkoxy, fluoro-lower-alkyl, fluoro-lower-alkoxy, hydroxyl-lower-alkyl, aryl, COOH, C(O)O-lower-alkyl or cyano; and pharmaceutically acceptable salts and esters thereof.

In some embodiments, the mitochondrial lipid shunt modulator of Formula (I) is a CPT1 inhibitor. In some embodiments, the mitochondrial lipid shunt modulator of Formula (I) is selected from the group consisting of:
4-{[4-(3-Chloro-benzenesulfonyl)-3,4-dihydro-2H-benzo[1,4]thiazine-6-carbonyl]-amino}-benzoic acid (1)
4-{[4-(3-Chloro-benzenesulfonyl)-1-oxo-1,2,3,4-tetrahydro-benzo[1,4]thiazine-6-carbonyl]-amino}-benzoic acid (2)
4-{[4-(3-Chloro-benzenesulfonyl)-1,1-dioxo-1,2,3,4-tetrahydro-benzo[1,4]thiazine-6-carbonyl]-amino}-benzoic acid (3)
4-{[4-(3,5-Dimethyl-benzenesulfonyl)-3,4-dihydro-2H-benzo[1,4]thiazine-6-carbonyl]amino}-benzoic acid (4)
4-{[4-(3,5-Dimethyl-benzenesulfonyl)-1-oxo-1,2,3,4-tetrahydro-benzo[1,4]thiazine-6-carbonyl]-amino}-benzoic acid (5)
4-{[4-(3,5-Dimethyl-benzenesulfonyl)-1,1-dioxo-1,2,3,4-tetrahydro-benzo[1,4]thiazine-6-carbonyl]-amino}-benzoic acid (6)
4-{[4-(5-Chloro-2-methoxy-benzenesulfonyl)-1-oxo-1,2,3,4-tetrahydro-benzo[1,4]thiazine-6-carbonyl]amino}-benzoic acid (7)
(4-{[4-(5-Chloro-2-methoxy-benzenesulfonyl)-1-oxo-1,2,3,4-tetrahydro-benzo[1,4]thiazine-6-carbonyl]-amino}-phenyl)-acetic acid,
(4-{[4-(5-Chloro-2-methoxy-benzenesulfonyl)-1,1-dioxo-1,2,3,4-tetrahydro-benzo[1,4]thiazine-6-carbonyl]amino}-phenyl)-acetic acid (8)
4-{[4-(5-Chloro-2-methoxy-benzenesulfonyl)-3,4-dihydro-2H-benzo[1,4]thiazine-6-carbonyl]-amino}-2-fluoro-benzoic acid (9)
4-{[4-(5-Chloro-2-methoxy-benzenesulfonyl)-1,1-dioxo-1,2,3,4-tetrahydro-benzo[1,4]thiazine-6-carbonyl]-amino}-2-fluoro-benzoic acid (10)
2-Chloro-4-{[4-(5-chloro-2-methoxy-benzenesulfonyl)-3,4-dihydro-2H-benzo[1,4]thiazine-6-carbonyl]-amino}-benzoic acid (11), and
2-Chloro-4-{[4-(5-chloro-2-methoxy-benzenesulfonyl)-1,1-dioxo-1,2,3,4-tetrahydro-benzo [1,4 thiazine-6-carbonyl]amino}-benzoic acid (12),
and pharmaceutically acceptable salts and esters thereof.

In some embodiments, the mitochondrial lipid shunt modulator is a compound disclosed in U.S. Pat. No. 7,713,996 (e.g., a compound of Formula (II) or a salt or derivative thereof), incorporated herein by reference in its entirety. In some embodiments, the mitochondrial lipid shunt modulator is a compound of Formula (II):

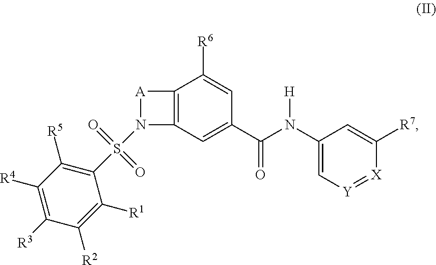

wherein X is N or CR⁸; Y is N or CR⁹; A is —C(R¹⁰R¹¹)C(R¹²R¹³)—, —C(R¹⁰R¹¹) C(R¹²R¹³) C(R¹⁴R¹⁵)—, —C(R¹⁰R¹¹)C(R¹²R¹³)C(R¹⁴R¹⁵)C(R¹⁶R¹⁷)—, C(R¹⁰R¹¹)C(R¹²R¹³)C(R¹⁴R¹⁵) C(R¹⁶R¹⁷)C(R¹⁸R¹⁹)— or —C(R¹¹)═C(R¹¹)—; R¹, R², R³, R⁴ and R⁵ independently from each other are hydrogen, halogen, cyano, hydroxy, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, NH₂—S(O)₂, N(H, lower-alkyl)-S(O)₂ or N(lower-alkyl)₂-S(O)₂, NH₂—C(O), N(H, lower-alkyl)-C(O), N(lower-alkyl)₂-C(O) or lower-alkoxy-C(O), wherein lower-alkyl is optionally substituted with hydroxy, lower-alkoxy, NH₂, N(H, lower-alkyl) or N(lower-alkyl)₂; R⁶ is hydrogen, halogen, lower-alkyl, lower-alkoxy, fluoro-lower-alkyl, fluoro-lower-alkoxy, hydroxy or hydroxy-lower-alkyl; R⁷ is hydrogen, halogen, hydroxy, cyano, lower-alkyl, lower-alkoxy, fluoro-lower-alkyl, fluoro-lower-alkoxy or hydroxy-lower-alkyl; R⁸ is hydrogen, halogen, cyano, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, NH₂—S(O)₂, N(H, lower-alkyl)-S(O)₂, N(lower-alkyl)₂-S(O)₂, NH₂—C(O), N(H, lower-alkyl)-C(O), N(lower-alkyl)₂-C(O), lower-alkoxy-C(O), COOH, 1H-tetrazolyl, 4H-[1,2,4]oxadiazol-3-yl-5-one, 4H-[1,2,4]thiadiazol-3-yl-5-one, 4H-[1,2,4]oxadiazol-3-yl-5-thione, 3H-[1,2,3,5]oxathiadiazol-4-yl-2-oxide, SO₃H, 3-hydroxy-isooxazolyl, 3-hydroxy-pyran-4-one-yl or P(OXOCH₂CH₃)OH, wherein lower-alkyl is optionally substituted with hydroxy, NH₂, N(H, lower-alkyl) or N(lower-alkyl)₂, and wherein fluoro-lower-alkyl is optionally substituted with hydroxy; R⁹ is hydrogen, halogen, hydroxy, cyano, lower-alkyl, lower-alkoxy, fluoro-lower-alkyl, fluoro-lower-alkoxy or hydroxy-lower-alkyl; R¹⁰, R¹¹, R¹², R¹³, R¹⁴, R¹⁵, R¹⁶, R¹⁷, R¹⁸, R¹⁹ independently from each other are hydrogen, halogen, hydroxy, lower-alkyl, lower-alkoxy, fluoro-lower-alkyl, fluoro-lower-alkoxy, hydroxy-lower-alkyl or cyano; and pharmaceutically acceptable salts and esters thereof.

In some embodiments, the mitochondrial lipid shunt modulator of Formula (II) is a CPT1 inhibitor. In some embodiments, the mitochondrial lipid shunt modulator of Formula (II) is selected from the group consisting of:
2-Chloro-4-{[1-(3-chloro-benzenesulfonyl)-2,3-dihydro-1H-indole-6-carbonyl]-amino}-benzoic acid (13)
2-Chloro-4-{[1-(5-chloro-2-methoxy-benzenesulfonyl)-2,3-dihydro-1H-indole-6-carbonyl]-amino}-benzoic acid (14)
2-Chloro-4-{[1-(3-trifluoromethyl-benzenesulfonyl)-2,3-dihydro-1H-indole-6-carbonyl]-amino}-benzoic acid (15)
2-Chloro-4-{[1-(3,4-dichloro-benzenesulfonyl)-2,3-dihydro-1H-indole-6-carbonyl]-amino}-benzoic acid (16)
2-Chloro-4-{[1-(3,5-dichloro-benzenesulfonyl)-2,3-dihydro-1H-indole-6-carbonyl]-amino}-benzoic acid (17)
2-Chloro-4-{[1-(3-fluoro-benzenesulfonyl)-2,3-dihydro-1H-indole-6-carbonyl]-amino}-benzoic acid (18)
2-Chloro-4-{[1-(2-methoxy-5-methyl-benzenesulfonyl)-2,3-dihydro-1H-indole-6-carbonyl]-amino}-benzoic acid (19)
2-Chloro-4-{[1-(4-difluoromethoxy-benzenesulfonyl)-2,3-dihydro-1H-indole-6-carbonyl]-amino}-benzoic acid (20)
4-{[1-(3-Chloro-benzenesulfonyl)-2,3-dihydro-1H-indole-6-carbonyl]-amino}-2-fluoro-benzoic acid (21)
2-Fluoro-4-{[1-(3-trifluoromethyl-benzenesulfonyl)-2,3-dihydro-1H-indole-6-carbonyl]-amino}-benzoic acid (22)
4-{[1-(3,4-Dichloro-benzenesulfonyl)-2,3-dihydro-1H-indole-6-carbonyl]-amino}-2-fluoro-benzoic acid (23)
4-{[1-(5-Chloro-2-methoxy-benzenesulfonyl)-2,3-dihydro-1H-indole-6-carbonyl]-amino}-2-fluoro-benzoic acid (24)
1-(3-Chloro-benzenesulfonyl)-2,3-dihydro-1H-indole-6-carboxylic acid [4-(1H-tetrazol-5-yl)-3-trifluoromethyl-phenyl]-amide (25)
2-Fluoro-4-{[4-methoxy-1-(3-trifluoromethyl-benzenesulfonyl)-2,3-dihydro-1H-indole-6-carbonyl]-amino}-benzoic acid (26)
4-{[1-(3-Chloro-benzenesulfonyl)-4-methoxy-2,3-dihydro-1H-indole-6-carbonyl]-amino}-2-fluoro-benzoic acid (27)
4-{[1-(3,4-Dichloro-benzenesulfonyl)-4-methoxy-2,3-dihydro-1H-indole-6-carbonyl]-amino}-2-fluoro-benzoic acid (28)
4-{[1-(5-Chloro-2-methoxy-benzenesulfonyl)-4-methoxy-2,3-dihydro-1H-indole-6-carbonyl]-amino}-2-fluoro-benzoic acid (29)
4-{[1-(3,5-Dichloro-benzenesulfonyl)-4-methoxy-2,3-dihydro-1H-indole-6-carbonyl]-amino}-2-fluoro-benzoic acid (30)
2-Fluoro-4-{[1-(3-fluoro-benzenesulfonyl)-4-methoxy-2,3-dihydro-1H-indole-6-carbonyl]-amino}-benzoic acid (31)
2-Fluoro-4-{[4-methoxy-1-(2-methoxy-5-methyl-benzenesulfonyl)-2,3-dihydro-1H-indole-6-carbonyl]-amino}-benzoic acid (32)
2-Chloro-4-{[4-methoxy-1-(3-trifluoromethyl-benzenesulfonyl)-2,3-dihydro-1H-indole-6-carbonyl]-amino}-benzoic acid (33)
2-Chloro-4-{[1-(5-chloro-2-methoxy-benzenesulfonyl)-4-methoxy-2,3-dihydro-1H-indole-6-carbonyl]-amino}-benzoic acid (34)
2-Chloro-4-{[1-(3,5-dichloro-benzenesulfonyl)-4-methoxy-2,3-dihydro-1H-indole-6-carbonyl]-amino}-benzoic acid (35)
2-Chloro-4-{[1-(3-fluoro-benzenesulfonyl)-4-methoxy-2,3-dihydro-1H-indole-6-carbonyl]-amino}-benzoic acid (36)
2-Chloro-4-{[1-(3-chloro-benzenesulfonyl)-4-methoxy-2,3-dihydro-1H-indole-6-carbonyl]-amino}-benzoic acid (37)
4-{[1-(3-Chloro-benzenesulfonyl)-4-methoxy-2,3-dihydro-1H-indole-6-carbonyl]-amino}-benzoic acid (38)
4-{[1-(5-Chloro-2-methoxy-benzenesulfonyl)-4-methoxy-2,3-dihydro-1H-indole-6-carbonyl]-amino}-benzoic acid (39)
4-{[4-Methoxy-1-(2-methoxy-5-methyl-benzenesulfonyl)-2,3-dihydro-1H-indole-6-carbonyl]-amino}-benzoic acid (40)
4-{[1-(3-Fluoro-benzenesulfonyl)-4-methoxy-2,3-dihydro-1H-indole-6-carbonyl]-amino}-benzoic acid (41)
4-{[1-(3,5-Dichloro-benzenesulfonyl)-2,3-dihydro-1H-indole-6-carbonyl]-amino}-benzoic acid (42)
4-{[1-(5-Chloro-2-methoxy-benzenesulfonyl)-2,3-dihydro-1H-indole-6-carbonyl]-amino}-benzoic acid (43)
4-{[1-(3-Trifluoromethoxy-benzenesulfonyl)-2,3-dihydro-1H-indole-6-carbonyl]-amino}-benzoic acid (44)
4-{[1-(3-Fluoro-benzenesulfonyl)-2,3-dihydro-1H-indole-6-carbonyl]-amino}-benzoic acid (45)
1-(5-Chloro-2-methoxy-benzenesulfonyl)-2,3-dihydro-1H-indole-6-carboxylic acid (4-chloro-phenyl)-amide (46)
1-(5-Chloro-2-methoxy-benzenesulfonyl)-2,3-dihydro-1H-indole-6-carboxylic acid phenylamide (47)
4-[(1-Benzenesulfonyl-2,3-dihydro-1H-indole-6-carbonyl)-amino]-benzoic acid (48)
1-(5-Chloro-2-methoxy-benzenesulfonyl)-2,3-dihydro-1H-indole-6-carboxylic acid [4-(1H-tetrazol-5-yl)-phenyl]-amide (49)
1-(5-Chloro-2-methoxy-benzenesulfonyl)-2,3-dihydro-1H-indole-6-carboxylic acid [4-(5-oxo-4,5-dihydro-[1,2,4]oxadiazol-3-yl)-phenyl]-amide (50)
1-(5-Chloro-2-methoxy-benzenesulfonyl)-2,3-dihydro-1H-indole-6-carboxylic acid [4-(2,2,2-trifluoro-1-hydroxy-ethyl)-phenyl]-amide (51)
2-Chloro-4-{[1-(3-fluoro-benzenesulfonyl)-1,2,3,4-tetrahydro-quinoline-7-carbonyl]-amino}-benzoic acid (52)
2-Chloro-4-{[1-(3,4-dichloro-benzenesulfonyl)-1,2,3,4-tetrahydro-quinoline-7-carbonyl]-amino}-benzoic acid (53)
2-Chloro-4-{[1-(3,5-dichloro-benzenesulfonyl)-1,2,3,4-tetrahydro-quinoline-7-carbonyl]-amino}-benzoic acid (54)
2-Chloro-4-{[1-(3-trifluoromethyl-benzenesulfonyl)-1,2,3,4-tetrahydro-quinoline-7-carbonyl]-amino}-benzoic acid (55)
2-Chloro-4-{[1-(4-difluoromethoxy-benzenesulfonyl)-1,2,3,4-tetrahydro-quinoline-7-carbonyl]-amino}-benzoic acid (56)
2-Chloro-4-{[1-(5-chloro-2-methoxy-benzenesulfonyl)-1,2,3,4-tetrahydro-quinoline-7-carbonyl]-amino}-benzoic acid (57)
2-Chloro-4-{[1-(2-methoxy-5-methyl-benzenesulfonyl)-1,2,3,4-tetrahydro-quinoline-7-carbonyl]-amino}-benzoic acid (58)
2-Chloro-4-{[1-(3-chloro-benzenesulfonyl)-1,2,3,4-tetrahydro-quinoline-7-carbonyl]-amino}-benzoic acid (59)
2-Fluoro-4-{[1-(3-fluoro-benzenesulfonyl)-1,2,3,4-tetrahydro-quinoline-7-carbonyl]-amino}-benzoic acid (60)
4-{[1-(3-Chloro-benzenesulfonyl)-1,2,3,4-tetrahydro-quinoline-7-carbonyl]-amino}-2-fluoro-benzoic acid (61)
2-Fluoro-4-{[1-(2-methoxy-5-methyl-benzenesulfonyl)-1,2,3,4-tetrahydro-quinoline-7-carbonyl]-amino}-benzoic acid (62)
4-{[1-(5-Chloro-2-methoxy-benzenesulfonyl)-1,2,3,4-tetrahydro-quinoline-7-carbonyl]-amino}-2-fluoro-benzoic acid (63)
4-{[1-(4-Difluoromethoxy-benzenesulfonyl)-1,2,3,4-tetrahydro-quinoline-7-carbonyl]-amino}-2-fluoro-benzoic acid (64)
4-{[1-(3,5-Dichloro-benzenesulfonyl)-1,2,3,4-tetrahydro-quinoline-7-carbonyl]-amino}-2-fluoro-benzoic acid (65)
2-Fluoro-4-{[1-(3-trifluoromethyl-benzenesulfonyl)-1,2,3,4-tetrahydro-quinoline-7-carbonyl]-amino}-benzoic acid (66)
4-{[1-(3,4-Dichloro-benzenesulfonyl)-1,2,3,4-tetrahydro-quinoline-7-carbonyl]-amino}-2-fluoro-benzoic acid (67)
1-(3-Chloro-benzenesulfonyl)-1,2,3,4-tetrahydro-quinoline-7-carboxylic acid [4-(1H-tetrazol-5-yl)-3-trifluoromethyl-phenyl]-amide (68)
4-{[1-(5-Chloro-2-methoxy-benzenesulfonyl)-1,2,3,4-tetrahydro-quinoline-7-carbonyl]-amino}-benzoic acid (69)
4-{[1-(3-Chloro-benzenesulfonyl)-1,2,3,4-tetrahydro-quinoline-7-carbonyl]-amino}-benzoic acid (70)
4-{[1-(3-Trifluoromethyl-benzenesulfonyl)-1,2,3,4-tetrahydro-quinoline-7-carbonyl]-amino}-benzoic acid (71)
4-{[1-(3-fluoro-benzenesulfonyl)-1,2,3,4-tetrahydro-quinoline-7-carbonyl]-amino}-benzoic acid (72)
4-{[1-(5-Chloro-2-methoxy-benzenesulfonyl)-1H-indole-6-carbonyl]-amino}-benzoic acid (73)
2-Chloro-4-{[1-(3,4-dichloro-benzenesulfonyl)-2,3-dihydro-1H-indole-6-carbonyl]-amino}-benzoic acid (74)
2-Chloro-4-{[1-(3,5-dichloro-benzenesulfonyl)-2,3-dihydro-1H-indole-6-carbonyl]-amino}-benzoic acid (75)
2-Fluoro-4-{[1-(3-trifluoromethyl-benzenesulfonyl)-2,3-dihydro-1H-indole-6-carbonyl]-amino}-benzoic acid (76)
4-{[1-(5-Chloro-2-methoxy-benzenesulfonyl)-2,3-dihydro-1H-indole-6-carbonyl]-amino}-benzoic acid (77)
1-(5-Chloro-2-methoxy-benzenesulfonyl)-2,3-dihydro-1H-indole-6-carboxylic acid [4-(1H-tetrazol-5-yl)-phenyl]-amide (78)
2-Chloro-4-{[1-(5-chloro-2-methoxy-benzenesulfonyl)-1,2,3,4-tetrahydro-quinoline-7-carbonyl]-amino}-benzoic acid (79)
2-Fluoro-4-{[1-(2-methoxy-5-methyl-benzenesulfonyl)-1,2,3,4-tetrahydro-quinoline-7-carbonyl]-amino}-benzoic acid (80)
4-{[1-(5-Chloro-2-methoxy-benzenesulfonyl)-1,2,3,4-tetrahydro-quinoline-7-carbonyl]-amino}-2-fluoro-benzoic acid (81)
4-{[1-(3,5-Dichloro-benzenesulfonyl)-1,2,3,4-tetrahydro-quinoline-7-carbonyl]-amino}-2-fluoro-benzoic acid (82)
4-{[1-(5-Chloro-2-methoxy-benzenesulfonyl)-1,2,3,4-tetrahydro-quinoline-7-carbonyl]-amino}-benzoic acid (83)
4-{[1-(5-Chloro-2-methoxy-benzenesulfonyl)-1H-indole-6-carbonyl]-amino}-benzoic acid (84)
4-{[1-(5-Chloro-2-methoxy-benzenesulfonyl)-4-methyl-2,3-dihydro-1H-indole-6-carbonyl]-amino}-benzoic acid (85)
4-{[1-(5-Chloro-2-methoxy-benzenesulfonyl)-4-methyl-2,3-dihydro-1H-indole-6-carbonyl]-amino}-2-fluoro-benzoic acid (86)
2-Chloro-4-{[1-(5-chloro-2-methoxy-benzenesulfonyl)-4-methyl-2,3-dihydro-1H-indole-6-carbonyl]-amino)benzoic acid (87)
4-{[1-(5-Chloro-2-methoxy-benzenesulfonyl)-2,3,4,5-tetrahydro-1H-benzo[b]azepine-8-carbonyl]-amino}-benzoic acid (88)
4-{[1-(5-Chloro-2-methoxy-benzenesulfonyl)-2,3,4,5-tetrahydro-1H-benzo[b]azepine-8-carbonyl]-amino}-2-fluoro-benzoic acid (89)
2-Chloro-4-{[1-(5-chloro-2-methoxy-benzenesulfonyl)-2,3,4,5-tetrahydro-1H-benzo[b]azepine-8-carbonyl]-amino}-benzoic acid (90)
4-{[1-(5-Chloro-2-methoxy-benzenesulfonyl)-3-methyl-2,3-dihydro-1H-indole-6-carbonyl]-amino}-benzoic acid (91)
4-{[1-(5-Chloro-2-methoxy-benzenesulfonyl)-3-methyl-2,3-dihydro-1H-indole-6-carbonyl]-amino}-2-fluoro-benzoic acid (92)
2-Chloro-4-{[1-(5-chloro-2-methoxy-benzenesulfonyl)-3-methyl-2,3-dihydro-1H-indole-6-carbonyl]-amino}-benzoic acid (93)
4-{[1-(2-Methoxy-5-methyl-benzenesulfonyl)-1H-indole-6-carbonyl]-amino}-benzoic acid (94)
4-{[1-(3-Chloro-benzenesulfonyl)-1H-indole-6-carbonyl]-amino}-benzoic acid (95); and
4-{[1-(3,5-Dimethyl-benzenesulfonyl)-1H-indole-6-carbonyl]-amino}-benzoic acid (96)
   and pharmaceutically acceptable salts and esters thereof.

In some embodiments, the mitochondrial lipid shunt modulator is a compound disclosed in U.S. Pat. No. 7,723,538 (e.g., a compound of Formula (III) or a salt or derivative thereof), incorporated herein by reference in its entirety. In some embodiments, the mitochondrial lipid shunt modulator is a compound of Formula (III):

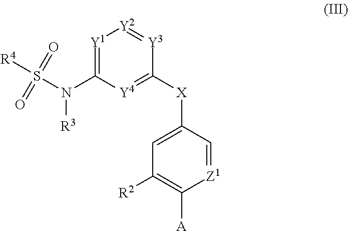

wherein A is —C(O)OR¹ or selected from the group consisting of tetrazol-5-yl, 5-thioxo-4,5-dihydro-[1,2,4]oxadiazol-3-yl, 2-oxo-2,3-dihydro-[1,2,3,5]oxathiadiazol-4-yl and 5-oxo-4,5-dihydro-[1,2,4]oxadiazol-3-yl; X is —N(R⁵)C(O)— or —C(O)N(R⁵)—; Y¹ is N or C(R6); Y² is N or C(R7); Y³ is N or C(H); Y⁴ is N or C(R⁸); Z¹ is N or C(R); R¹ is hydrogen or lower-alkyl; R² is hydrogen, halogen, hydroxy, cyano, lower-alkyl, fluoro-lower-alkyl, lower-alkoxy, fluoro-lower-alkoxy; NH₂, N(H,lower-alkyl), N(lower-alkyl)₂, or lower-alkyl-C(O)—O—, wherein lower-alkyl is optionally substituted with hydroxy, halogen, NH₂, N(H,lower-alkyl), N(lower-alkyl)₂ or lower-alkoxy; R³ is hydrogen, lower-alkyl or lower-alkoxy-lower-alkyl; R⁴ is aryl or heteroaryl, which aryl or heteroaryl is optionally substituted with to 3 substituents independently selected from the group consisting of halogen, cyano, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, S(O)₂, N(H,lower-alkyl)-S(O)₂, N(lower-alkyl)₂-S(O)₂, NH₂—C(O), N(H,lower-alkyl)-C(O), N(lower-alkyl)₂-C(O), lower-alkoxy-C(O) and heteroaryl which is optionally substituted with lower-alkyl, halogen, thio-lower-alkoxy, or fluoro-lower-alkyl, wherein lower-alkyl is optionally substituted with hydroxy, NH₂, N(H,lower-alkyl) or N(lower-alkyl)₂; R⁵ is hydrogen, lower-alkyl or lower-alkoxy-lower-alkyl; R⁶, R⁷ and R⁸ independently from each other are selected from the group consisting of hydrogen, halogen, hydroxy, cyano, lower-alkyl, fluoro-lower-alkyl, lower-alkoxy, fluoro-lower-alkoxy; NH₂, N(H,lower-alkyl), N(lower-alkyl)₂, and lower-alkyl-C(O)—O—, wherein lower-alkyl is optionally substituted with hydroxy, halogen, NH₂, N(H,lower-alkyl), N(lower-alkyl)₂ or lower-alkoxy; R⁹ is hydrogen, halogen, hydroxy, cyano, lower-alkyl, fluoro-lower-alkyl, lower-alkoxy, fluoro-lower-alkoxy; NH₂, N(H,lower-alkyl), N(lower-alkyl)₂, or lower-alkyl-C(O)—O—, wherein lower-alkyl is optionally substituted with hydroxy, halogen, NH₂, N(H,lower-alkyl), N(lower-alkyl)₂ or lower-alkoxy; or a pharmaceutically acceptable salt or ester thereof.

In some embodiments, the mitochondrial lipid shunt modulator of Formula (III) is a CPT1 inhibitor. In some embodiments, the mitochondrial lipid shunt modulator of Formula (III) is selected from the group consisting of:
4-(3-Benzenesulfonylamino-benzoylamino)-benzoic acid (97)
4-[3-(4-Methoxy-benzenesulfonylamino)-benzoylamino]-benzoic acid (98)
4-[3-(3-Fluoro-benzenesulfonylamino)-benzoylamino]-benzoic acid (99)
4-[3-(Naphthalene-2-sulfonylamino)-benzoylamino]-benzoic acid (100)
4-[3-(3,4-Dichloro-benzenesulfonylamino)-benzoylamino]-benzoic acid (101)
4-[3-(2-Methoxy-benzenesulfonylamino)-benzoylamino]-benzoic acid (102)
4-[3-(3-Methoxy-benzenesulfonylamino)-benzoylamino]-benzoic acid (103)
4-[3-(2-Fluoro-benzenesulfonylamino)-benzoylamino]-benzoic acid (104)
4-[3-(3-Chloro-benzenesulfonylamino)-benzoylamino]-benzoic acid (105)
4-[3-(3-Trifluoromethyl-benzenesulfonylamino)-benzoylamino]-benzoic acid (106)
4-[3-(3,5-Dichloro-benzenesulfonylamino)-benzoylamino]-benzoic acid (107)
4-[3-(5-Chloro-2-methoxy-benzenesulfonylamino)-benzoylamino]-benzoic acid (108)
4-[3-(3-Difluoromethoxy-benzenesulfonylamino)-benzoylamino]-benzoic acid (109)
4-[3-(5-Isoxazol-3-yl-thiophene-2-sulfonylamino)-benzoylamino]-benzoic acid (110)
4-{3-[5-(2-Methylsulfanyl-pyrimidin-4-yl)-thiophene-2-sulfonylamino]-benzoylamino}-benzoic acid (111)
4-{3-[5-(2-Methyl-5-trifluoromethyl-2H-pyrazol-3-yl)-thiophene-2-sulfonylamino]-benzoylamino)-benzoic acid (112)
2-Methoxy-4-[3-(3-trifluoromethyl-benzenesulfonylamino)-benzoylamino]-benzoic acid (113)
4-[3-(3,5-Dichloro-benzenesulfonylamino)-benzoylamino]-2-methoxy-benzoic acid (114)
4-[3-(3,4-Dichloro-benzenesulfonylamino)-benzoylamino]-2-methoxy-benzoic acid (115)
4-[3-(3-Chloro-benzenesulfonylamino)-benzoylamino]-2-methoxy-benzoic acid (116)
4-[4-Chloro-3-(3-chloro-benzenesulfonylamino)-benzoylamino]-benzoic acid (117)
4-[4-Chloro-3-(3,5-dichloro-benzenesulfonylamino)-benzoylamino]-benzoic acid (118)
4-[4-Chloro-3-(3-trifluoromethyl-benzenesulfonylamino)-benzoylamino]-benzoic acid (119)
4-[4-Chloro-3-(3-trifluoromethoxy-benzenesulfonylamino)-benzoylamino]-benzoic acid (120)
4-[4-Chloro-3-(5-chloro-2-methoxy-benzenesulfonylamino)-benzoylamino]-benzoic acid (121)
4-[4-Chloro-3-(3,4-dichloro-benzenesulfonylamino)-benzoylamino]-benzoic acid (122)
4-[4-Chloro-3-(benzenesulfonylamino)-benzoylamino]-benzoic acid (123)
4-[4-Chloro-3-(2,5-dichloro-benzenesulfonylamino)-benzoylamino]-benzoic acid (124)
4-[4-Chloro-3-(3-fluoro-benzenesulfonylamino)-benzoylamino]-benzoic acid (125)
4-[4-Chloro-3-(2,5-dimethoxy-benzenesulfonylamino)-benzoylamino]-benzoic acid (126)
4-[3-(3-Chloro-benzenesulfonylamino)-4,5-dimethoxy-benzoylamino]-benzoic acid (127)
4-[3-(3,5-Dichloro-benzenesulfonylamino)-4,5-dimethoxy-benzoylamino]-benzoic acid (128)
4-[3,4-Dimethoxy-5-(3-trifluoromethyl-benzenesulfonylamino)-benzoylamino]-benzoic acid (129)
4-[3-(5-Chloro-2-methoxy-benzenesulfonylamino)-4,5-dimethoxy-benzoylamino]-benzoic acid (130)
4-[3-(3,4-Dichloro-benzenesulfonylamino)-4,5-dimethoxy-benzoylamino]-benzoic acid (131)
4-[3-(benzenesulfonylamino)-4,5-dimethoxy-benzoylamino]-benzoic acid (132)
4-[3-(2,5-Dichloro-benzenesulfonylamino)-4,5-dimethoxy-benzoylamino]-benzoic acid (133)
4-[3-(3-Chloro-benzenesulfonylamino)-benzoylamino]-2-fluoro-benzoic acid (134)
5-[3-(3-Chloro-benzenesulfonylamino)-benzoylamino]-pyridine-2-carboxylic acid (135)
4-[3-(3,4-Dichloro-benzenesulfonylamino)-benzoylamino]-benzoic acid (136)
4-[3-(3-Chloro-benzenesulfonylamino)-benzoylamino]-benzoic acid (137)
4-[3-(3,5-Dichloro-benzenesulfonylamino)-benzoylamino]-benzoic acid (138)
4-[3-(5-Chloro-2-methoxy-benzenesulfonylamino)-benzoylamino]-benzoic acid (139)
4-[4-Chloro-3-(3-chloro-benzenesulfonylamino)-benzoylamino]-benzoic acid (140)
4-[4-Chloro-3-(3,5-dichloro-benzenesulfonylamino)-benzoylamino]-benzoic acid (141)
4-[4-Chloro-3-(5-chloro-2-methoxy-benzenesulfonylamino)-benzoylamino]-benzoic acid (142)
4-[3-(3,5-Dichloro-benzenesulfonylamino)-4,5-dimethoxy-benzoylamino]-benzoic acid (143)
4-[3-(5-Chloro-2-methoxy-benzenesulfonylamino)-4,5-dimethoxy-benzoylamino]-benzoic acid (144)
3-(3-Chloro-benzenesulfonylamino)-[4-(tetrazol-5-yl)-phenyl]-benzamide (145)
3-(5-Chloro-2-methoxy-benzenesulfonylamino)-[4-(tetrazol-5-yl)-phenyl]-benzamide (146)
3-(5-Chloro-2-methoxy-benzenesulfonylamino)-N-[4-(5-thioxo-4,5-dihydro-[1,2,4]oxadiazol-3-yl)-phenyl]-benzamide (147)
3-(5-Chloro-2-methoxy-benzenesulfonylamino)-N-[4-(2-oxo-2,3-dihydro-[1,2,3,5]oxathiadiazol-4-yl)-phenyl]-benzamide (148), and
3-(5-Chloro-2-methoxy-benzenesulfonylamino)-N-[4-(5-oxo-4,5-dihydro-[1,2,4]oxadiazol-3-yl)-phenyl]-benzamide (149)
   and a pharmaceutically acceptable salts and esters thereof.

In some embodiments, the mitochondrial lipid shunt modulator is a compound as disclosed in U.S. Pat. No. 7,858,645 (e.g., a compound of Formula (IV) or a salt or derivative thereof), incorporated herein by reference in its entirety. In some embodiments, the mitochondrial lipid shunt modulator is a compound of Formula (IV):

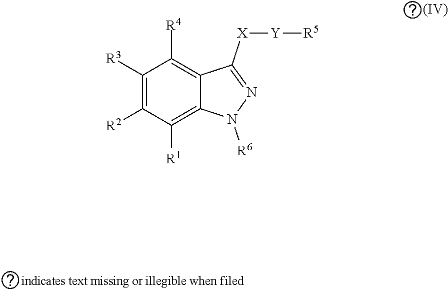

wherein X is —C(O)—NH—, —NH—C(O)— or —(CR⁷R⁸)ₘ—S—; Y is —(CR⁹R¹⁰)ₙ—; R¹, R², R³ and R⁴ are hydrogen independently of each other, halogen, alkyl, fluoro-alkyl, alkoxyl group, fluoro-alkoxyl group, formamyl, -alkyl-NH—C(O)—NH, aryl-alkyl-NH—C(O)—NH or alkyl-SO²—NH— is rudimentary-alkyl; R⁵ is phenyl or heteroaryl, described heteroaryl is selected from thienyl, pyridyl, furyl and thiazolyl, described phenyl or heteroaryl are optionally replaced by 1 to 3 substitutions, wherein each substitution is independently selected from halogen, and alkyl, fluoro-alkyl, alkoxyl group, fluoro-alkoxyl group, hydroxyl, HO—SO₂, NH₂—SO₂, N(H, alkyl)-SO₂, N (alkyl)₂-SO₂, -alkyl-SO₂—NH, carboxyl, carboxyl-alkyl, carboxyl-alkoxyl group, NO₂, CN, NH₂, N (H, alkyl), N(alkyl)₂, NH₂C (O), N (H, alkyl) C(O), N(alkyl)₂C(O), -alkyl-C(O) NH, 1H-tetrazolium-5-base and 5-oxo-4,5-dihydro-[1,2,4] oxadiazole-3-bases; R⁶ is C₂₋₇-alkyl or R⁷ and R⁸ are hydrogen independently of each other, alkyl, fluoro-alkyl, alkoxyl group or fluoro-alkoxyl group; R⁹ and R¹⁰ is hydrogen independently of each other, low alkyl group, fluoro-alkyl, alkoxyl group or fluoro-alkoxyl group; R¹¹ and R¹² are hydrogen independently of each other, -alkyl, fluoro-alkyl, alkoxyl group or fluoro-alkoxyl group; R¹³, R¹⁴ and R¹⁵ are hydrogen independently of each other, alkyl, halogen, fluoro-alkyl, alkoxyl group, hydroxyl, fluoro-alkoxyl group, NO₂ or NH₂—C(O); R¹⁶ is hydrogen or alkoxyl group; M is 0 or 1; N is 0 or 1; and pharmaceutical salts and ester thereof.

In some embodiments, the mitochondrial lipid shunt modulator of Formula (IV) is a CPT1 inhibitor. In some embodiments, the mitochondrial lipid shunt modulator of Formula (IV) is selected from the group consisting of:
N-[1-(4-difluoro-methoxy-benzyl)-1H-indazole-3-yl]-to phthalamic acid (150)
N-[1-(4-difluoro-methoxy-benzyl)-1H-indazole-3-yl]-benzamide (151)
Thiophene-2-carboxylic acid [1-(4-difluoro-methoxy-benzyl)-1H-indazole-3-yl]-acid amide (152)
N-[1-(4-difluoro-methoxy-benzyl)-1H-indazole-3-yl]-3-fluoro-benzamide (153)
N-[1-(4-difluoro-methoxy-benzyl)-1H-indazole-3-yl]-3-methoxyl group-benzamide (154)
N-[1-(4-difluoro-methoxy-benzyl)-1H-indazole-3-yl]-Isonicotinamide (155)
N-[1-(4-difluoro-methoxy-benzyl)-5-fluoro-1H-indazole-3-yl]-phthalamic acid (156)
N-[1-(4-difluoro-methoxy-benzyl)-5-fluoro-1H-indazole-3-yl]-benzamide (157)
N-[1-(4-difluoro-methoxy-benzyl)-5-fluoro-1H-indazole-3-yl]-3-fluoro-benzamide (158)
Thiophene-2-carboxylic acid [1-(4-difluoro-methoxy-benzyl)-5-fluoro-1H-indazole-3-yl]-acid amide (159)
N-[1-(4-difluoro-methoxy-benzyl)-5-fluoro-1H-indazole-3-yl]-3-methoxyl group-benzamide (160)
N-[1-(4-difluoro-methoxy-benzyl)-5-fluoro-1H-indazole-3-yl]-Isonicotinamide (161)
N-[1-(4-difluoro-methoxy-benzyl)-5-fluoro-1H-indazole-3-yl]-niacinamide (162)
N-[1-(3-chloro-benzyl)-5-fluoro-1H-indazole-3-yl]-phthalamic acid (163)
N-[1-(3-chloro-benzyl)-5-fluoro-1H-indazole-3-yl]-benzamide (164)
N-[1-(3-chloro-benzyl)-5-fluoro-1H-indazole-3-yl]-3-fluoro-benzamide (165)
N-[1-(3-chloro-benzyl)-5-fluoro-1H-indazole-3-yl]-3-methoxyl group-benzamide (166)
Thiophene-2-carboxylic acid [1-(3-chloro-benzyl)-5-fluoro-1H-indazole-3-yl]-acid amide (167)
Furan-2-carboxylic acid [1-(3-chloro-benzyl)-5-fluoro-1H-indazole-3-yl]-acid amide (168)
N-[1-(4-difluoro-methoxy-benzyl)-7-fluoro-1H-indazole-3-yl]-to phthalamic acid (169)
N-[1-(3-chloro-benzyl)-7-fluoro-1H-indazole-3-yl]-phthalamic acid (170)
Thiophene-2-carboxylic acid [1-(3-chloro-benzyl)-7-fluoro-1H-indazole-3-yl]-acid amide (171)
N-[1-(3-chloro-benzyl)-6-fluoro-1H-indazole-3-yl]-phthalamic acid (172)
Thiophene-2-carboxylic acid [1-(4-difluoro-methoxy-benzyl)-6-fluoro-1H-indazole-3-yl]-acid amide (173)
Thiophene-2-carboxylic acid (1-benzyl-1H-indazole-3-yl)-acid amide (174)
1-ethyl-5-(3-propyl group-urea groups)-1H-indazole-3-carboxylic acid (thiophene-2-ylmethyl)-acid amide (175)
1-ethyl-5-(3-sec-propyl-urea groups)-1H-indazole-3-carboxylic acid (thiophene-2-ylmethyl)-acid amide (176)
1-ethyl-5-(3-styroyl-urea groups)-1H-indazole-3-carboxylic acid (thiophene-2-ylmethyl)-acid amide (177)
1-ethyl-5-(3-ethyl-urea groups)-1H-indazole-3-carboxylic acid (thiophene-2-ylmethyl)-acid amide (178)
1-(4-nitro-benzyl)-1H-indazole-3-carboxylic acid (pyridin-3-yl methyl)-acid amide (179)
1-(4-difluoro-methoxy-benzyl)-1H-indazole-3-carboxylic acid (thiophene-2-ylmethyl)-acid amide (180)
1-(3-trifluoromethyl-benzyl)-1H-indazole-3-carboxylic acid (thiophene-2-ylmethyl)-acid amide (181)
4-({[1-(4-difluoro-methoxy-benzyl)-5-fluoro-1H-indazole-3-carbonyl]-amino}-methyl)-phenylformic acid (182)
(2-{[1-(4-difluoro-methoxy-benzyl)-5-fluoro-1H-indazole-3-carbonyl]-amino}-thiazole-4-yl)-acetate (183)
1-(4-difluoro-methoxy-benzyl)-5-fluoro-1H-indazole-3-carboxylic acid [4-(1H-tetrazolium-5-yl)-phenyl]-acid amide (184)
1-(4-difluoro-methoxy-benzyl)-5-fluoro-1H-indazole-3-carboxylic acid 4-(1H-tetrazolium-5-yl)-benzyl acid amide (185)
(4-{[1-(4-difluoro-methoxy-benzyl)-5-fluoro-1H-indazole-3-carbonyl]-amino}-2-methyl-phenoxy group)-acetate (186)
1-(4-difluoro-methoxy-benzyl)-5-fluoro-1H-indazole-3-carboxylic acid [4-(5-oxo-4, the 5-dihydro-[1,2,4] oxadiazole-3-yls)-phenyl]-acid amide (187)
4-{[5-fluoro-1-(3-trifluoromethyl-benzyl)-1H-indazole-3-carbonyl]-amino}-phenylformic acid (188)
5-fluoro-1-(3-trifluoromethyl-benzyl)-1H-indazole-3-carboxylic acid [4-(1H-tetrazolium-5-yl)-phenyl]-acid amide (189)
5-fluoro-1-(3-trifluoromethyl-benzyl)-1H-indazole-3-carboxylic acid 4-(1H-tetrazolium-5-yl)-benzyl acid amide (190)
1-(4-formamyl-benzyl)-1H-indazole-3-carboxylic acid 4-(1H-tetrazolium-5-yl)-benzyl acid amide (191)
1-(4-formamyl-benzyl)-5-fluoro-1H-indazole-3-carboxylic acid 4-(1H-tetrazolium-5-yl)-benzyl acid amide (192)
1-(5-chloro-2-methoxyl group-benzyl)-5-fluoro-3-[4-(1H-tetrazolium-5-yl)-thiophenyl methyl]-the 1H-indazole (193)
1-(4-difluoro-methoxy-benzyl)-1H-indazole-3,6-dicarboxylic acid 6-acid amides 3-[4-(1H-tetrazolium-5-yl)-benzyl acid amide] (194)
1-(4-difluoro-methoxy-benzyl)-6-(methylsulfonyl amino-methyl)-1H-indazole-3-carboxylic acid (195)
[4-(1H-tetrazolium-5-yl)-phenyl]-acid amide (196)
N-[1-(4-difluoro-methoxy-benzyl)-1H-indazole-3-yl]-phthalamic acid (197)
N-[1-(4-difluoro-methoxy-benzyl)-5-fluoro-1H-indazole-3-yl]-phthalamic acid (198)
N-[1-(4-difluoro-methoxy-benzyl)-5-fluoro-1H-indazole-3-yl]-benzamide (199)
N-[1-(4-difluoro-methoxy-benzyl)-5-fluoro-1H-indazole-3-yl]-3-fluoro-benzamide (200)
Thiophene-2-carboxylic acid [1-(4-difluoro-methoxy-benzyl)-5-fluoro-1H-indazole-3-yl]-acid amide (201)
N-[1-(4-difluoro-methoxy-benzyl)-5-fluoro-1H-indazole-3-yl]-3-methoxyl group-benzamide (202)
N-[1-(3-chloro-benzyl)-5-fluoro-1H-indazole-3-yl]-phthalamic acid (203)
Thiophene-2-carboxylic acid [1-(3-chloro-benzyl)-5-fluoro-1H-indazole-3-yl]-acid amide (204)
1-(3-trifluoromethyl-benzyl)-1H-indazole-3-carboxylic acid (thiophene-2-ylmethyl)-acid amide (205)
1-(4-formamyl-benzyl)-1H-indazole-3-carboxylic acid 4-(1H-tetrazolium-5-yl)-benzyl acid amide (206)
   and pharmaceutical salts and esters thereof.

In some embodiments, the mitochondrial lipid shunt modulator is a compound as disclosed in U.S. Pat. No. 7,645,776 (e.g., a compound of Formula (V) or a salt or derivative thereof), incorporated herein by reference in its entirety. In some embodiments, the mitochondrial lipid shunt modulator is a compound of Formula (V):

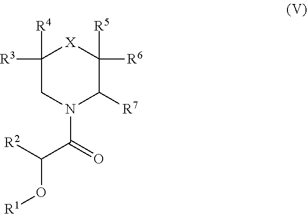

wherein X is C(R⁸R⁹) or NR¹⁰; R¹ is optionally phenyl that is replaced by 1 to 3 substituents, wherein each substituent is selected from halogen, hydroxyl, C₁₋₇-alkyl, hydroxyl-C₁₋₇-alkyl, and C₁₋₇-alkoxyl group; R² is hydrogen or C₁₋₇-alkyl; R³ and R⁴ are independently hydrogen, halogen, C₁₋₇-alkyl or C₁₋₇-alkoxyl; R⁵ and R⁶ are independently hydrogen, halogen, C₁₋₇-alkyl or C₁₋₇-alkoxyl; R⁷ is oxadiazolyl or triazolyl, wherein oxadiazolyl or triazolyl are substituted by R¹ or R¹²; R⁸ and R⁹ are independently hydrogen, halogen, hydroxyl, C₁₋₇-alkyl, or C₁₋₇-alkoxyl; R¹⁰ is hydrogen, or C₁₋₇-alkyl; R¹¹ is aryl, wherein aryl is optionally substituted with 1 to 3 substituents, wherein each substituent is selected from C₁₋₇-alkyl, hydroxyl, hydroxyl-C₁₋₇-alkyl, fluoro-C₁₋₇-alkyl, C₁₋₇-alkoxyl, halogen, N₂, and NR¹¹; R¹² is hydrogen or C₁₋₇-alkyl; R¹⁵ and R¹⁶ are independently hydrogen or aryl; and pharmaceutical salts and esters thereof.

In some embodiments, the mitochondrial lipid shunt modulator of Formula (V) is a CPT1 inhibitor. In some embodiments, the mitochondrial lipid shunt modulator of Formula (V) is selected from the group consisting of:
(R)-1-{2-[3-(4-methoxyl group-phenyl)-[1,2,4] oxadiazole-5-yl]-piperidin-1-yl}-2-phenoxy group-ethyl ketone (207)
(R)-1-{2-[3-(4-methoxyl group-phenyl)-[1,2,4] oxadiazole-5-yl]-piperidin-1-yl}-2-phenoxy group-third-1-ketone (208)
(R)-1-{2-[3-(4-bromo-phenyl) [1,2,4] oxadiazole-5-yl]-piperidin-1-yl}-2-phenoxy group-ethyl ketone (209)
(R)-2-(4-hydroxyl-phenoxy group)-1-{2-[3-(4-methoxyl group-phenyl)-[1,2,4]oxadiazole-5-yl]-piperidin-1-yl}-ethyl ketone (210)
(R)-2-(4-chloro-phenoxy group)-1-{2-[3-(4-methoxyl group-phenyl)-[1,2,4] oxadiazole-5-yl]-piperidin-1-yl}-ethyl ketone (211)
(R)-2-(4-methylol-phenoxy group)-1-{2-[3-(4-methoxyl group-phenyl)-[1,2,4]oxadiazole-5-yl]-piperidin-1-yl}-ethyl ketone (212)
(R)-2-(3-chloro-phenoxy group)-1-{2-[3-(4-methoxyl group-phenyl)-[1,2,4] oxadiazole-5-yl]-piperidin-1-yl}-ethyl ketone (213)
(R)-2-(4-fluoro-phenoxy group)-1-{2-[3-(4-methoxyl group-phenyl)-[1,2,4] oxadiazole-5-yl]-piperidin-1-yl}-ethyl ketone (214)
(R)-1-{2-[3-(4-fluoro-phenyl)-[1,2,4] oxadiazole-5-yl]-piperidin-1-yl}-2-phenoxy group-ethyl ketone (215)
(R)-1-{2-[3-(3-nitro-phenyl)-[1,2,4] oxadiazole-5-yl]-piperidin-1-yl}-2-phenoxy group-ethyl ketone (216),
(R)-1-{2-[3-(4-nitro-phenyl)-[1,2,4] oxadiazole-5-yl]-piperidin-1-yl}-2-phenoxy group-ethyl ketone (217),
(R)-1-{2-[3-(3-methylol-phenyl)-[1,2,4] oxadiazole-5-yl]-piperidin-1-yl}-2-phenoxy group-ethyl ketone (218)
(R)-2-phenoxy group-1-{2-[3-(4-trifluoromethyl-phenyl)-[1,2,4] oxadiazole-5-yl]-piperidin-1-yl}-ethyl ketone (219)
(R)-1-{2-[3-(4-chloro-phenyl)-[1,2,4] oxadiazole-5-yl]-piperidin-1-yl}-2-phenoxy group-ethyl ketone (220)
(R)-1-{2-[3-(4-methyl-3-nitro-phenyl)-[1,2,4] oxadiazole-5-yl]-piperidin-1-yl}-2-phenoxy group-ethyl ketone (221)
(R)-1-{2-[3-(4-methoxyl group-3-nitro-phenyl)-[1,2,4] oxadiazole-5-yl]-piperidin-1-yl}-2-phenoxy group-ethyl ketone (222)
(R)-1-{2-[3-(4-fluoro-3-trifluoromethyl-phenyl)-[1,2,4] oxadiazole-5-yl]-piperidin-1-yl}-2-phenoxy group-ethyl ketone (223)
(R)-2-phenoxy group-1-[2-(5-phenyl-2H-[1,2,4] triazole-3-yl)-piperidin-1-yl]-ethyl ketone (224)
(R)- and 1-{2-[5-(3,4-dimethoxy-phenyl)-2H-[1,2,4] triazole-3-yl]-piperidin-1-yl}-2-phenoxy group-ethyl ketone (225)
(R)- and 1-{2-[5-(3,4-, two chloro-phenyl)-2H-[1,2,4] triazole-3-yl]-piperidin-1-yl}-2-phenoxy group ethyl ketone (226)
(R)- and 1-{2-[5-(4-fluoro-phenyl)-2H-[1,2,4] triazole-3-yl]-piperidin-1-yl}-2-phenoxy group-ethyl ketone (227)
(R)- and 2-phenoxy group-1-{2-[5-(3-trifluoromethyl-phenyl)-2H-[1,2,4] triazole-3-yl]-piperidin-1-yl}-ethyl ketone (228)
(R)- and 1-{2-[5-(4-methoxyl group-phenyl)-2H-[1,2,4] triazole-3-yl]-piperidin-1-yl}-2-phenoxy group-ethyl ketone (229)
(R)- and 1-{2-[5-(3-nitro-phenyl)-2H-[1,2,4] triazole-3-yl]-piperidin-1-yl}-2-phenoxy group-ethyl ketone (230)
(R)- and 1-{2-[5-(4-fluoro-3-trifluoromethyl-phenyl)-2H-[1,2,4] triazole-3-yl]-piperidin-1-yl}-2-phenoxy group-ethyl ketone (231)
1-{2-[3-(4-methoxyl group-phenyl)-[1,2,4] oxadiazole-5-yl]-piperazine-1-yl}-2-phenoxy group-ethyl ketone (232)
1-{2-[3-(2,4-, two chloro-phenyl)-[1,2,4] oxadiazole-5-yl]-piperazine-1-yl}-2-phenoxy group-ethyl ketone (233)
1-{2-[3-(4-nitro-phenyl)-[1,2,4] oxadiazole-5-yl]-piperazine-1-yl}-2-phenoxy group-ethyl ketone (234)
1-{2-[3-(3-methylol-phenyl)-[1,2,4] oxadiazole-5-yl]-piperazine-1-yl}-2-phenoxy group-ethyl ketone (235)
1-{2-[3-(4-chloro-phenyl)-[1,2,4] oxadiazole-5-yl]-piperazine-1-yl}-2-phenoxy group-ethyl ketone (236)
1-{2-[3-(4-methyl-3-nitro-phenyl)-[1,2,4] oxadiazole-5-yl]-piperazine-1-yl}-2-phenoxy group-ethyl ketone (237)
1-{2-[3-(4-methoxyl group-3-nitro-phenyl)-[1,2,4] oxadiazole-5-yl]-piperazine-1-yl}-2-phenoxy group-ethyl ketone (238)
1-{2-[3-(4-chloro-3-nitro-phenyl)-[1,2,4] oxadiazole-5-yl]-piperazine-1-yl}-2-phenoxy group-ethyl ketone (239)
(R)-1-{2-[3-(4-methyl-3-nitro-phenyl)-[1,2,4] oxadiazole-5-yl]-piperazine-1-yl}-2-phenoxy group-ethyl ketone (240)
(R)-1-{2-[3-(3-nitro-phenyl)-[1,2,4] oxadiazole-5-yl]-piperazine-1-yl}-2-phenoxy group-ethyl ketone (241)
(R)-1-{2-[3-(4-fluoro-phenyl)-[1,2,4] oxadiazole-5-yl]-piperazine-1-yl}-2-phenoxy group-ethyl ketone (242)
(R)- and 1-{2-[5-(4-fluoro-phenyl)-2H-[1,2,4] triazole-3-yl]-piperazine-1-yl}-2-phenoxy group-ethyl ketone (243)
(R)- and 2-phenoxy group-1-{2-[5-(3-trifluoromethyl-phenyl)-2H-[1,2,4] triazole-3-yl]-piperazine-1-yl}-ethyl ketone (244)
1-{2-[5-(4-fluoro-3-trifluoromethyl-phenyl)-2H-[1,2,4] triazole-3-yl]-piperazine-1-yl}-2-phenoxy group-ethyl ketone (245)
2-phenoxy group-1-{2-[5-(3-trifluoromethyl-phenyl)-2H-[1,2,4] triazole-3-yl]-piperazine-1-yl}-ethyl ketone (246)
2-phenoxy group-1-[2-(the p-tolyl-2H-[1 of 5-, 2,4] triazole-3-yl)-piperazine-1-yl]-ethyl ketone (247)
2-phenoxy group-1-{2-[5-(4-trifluoromethyl-phenyl)-2H-[1,2,4] triazole-3-yl]-piperazine-1-yl}-ethyl ketone (248)
1-{2-[5-(4-methoxyl group-phenyl)-2H-[1,2,4] triazole-3-yl]-piperazine-1-yl}-2-phenoxy group-ethyl ketone (249)
1-{2-[5-(4-fluoro-phenyl)-2H-[1,2,4] triazole-3-yl]-piperazine-1-yl}-2-phenoxy group-ethyl ketone (250)
1-{2-[5-(4-fluoro-phenyl)-2H-[1,2,4] triazole-3-yl]-piperazine-1-yl}-2-phenoxy group-ethyl ketone (251)
1-{2-[5-(3,4-dimethoxy-phenyl)-2H-[1,2,4] triazole-3-yl]-piperazine-1-yl}-2-phenoxy group-ethyl ketone (252)
1-{2-[5-(3,4-, two chloro-phenyl)-2H-[1,2,4] triazole-3-yl]-piperazine-1-yl}-2-phenoxy group-ethyl ketone (253)
1-{2-[5-(2-fluoro-phenyl)-2H-[1,2,4] triazole-3-yl]-piperazine-1-yl}-2-phenoxy group-ethyl ketone (254)
(R)-1-{2-[3-(4-fluoro-phenyl)-[1,2,4] oxadiazole-5-yl]-4-methyl-piperazine-1-yl}-2-phenoxy group-ethyl ketone (255)
(R)- and 1-{4-methyl-2-[5-(3-trifluoromethyl-phenyl)-2H-[1,2,4] triazole-3-yl]-piperazine-1-yl}-2-phenoxy group-ethyl ketone (256)
(R)- and 1-{2-[5-(4-methoxyl group-phenyl)-2H-[1,2,4] triazole-3-yl]-4-methyl-piperazine-1-yl}-2-phenoxy group-ethyl ketone (257)
(R)- and 1-{2-[5-(4-fluoro-phenyl)-2H-[1,2,4] triazole-3-yl]-4-methyl-piperazine-1-yl}-2-phenoxy group-ethyl ketone (258)
(R)-1-{2-[3-(3-amino-phenyl)-[1,2,4] oxadiazole-5-yl]-piperidin-1-yl}-2-phenoxy group-ethyl ketone (259)
(R)-1-{2-[3-(4-amino-phenyl)-[1,2,4] oxadiazole-5-yl]-piperidin-1-yl}-2-phenoxy group-ethyl ketone (260)
(R)- and 1-{2-[5-(3-amino-phenyl)-2H-[1,2,4] triazole-3-yl]-piperidin-1-yl}-2-phenoxy group-ethyl ketone (261)
(R)- and 1-{2-[5-(4-fluoro-phenyl)-2H-[1,2,4] triazole-3-yl]-piperidin-1-yl}-2-phenoxy group-ethyl ketone (262)
(R)- and 2-phenoxy group-1-{2-[5-(3-trifluoromethyl-phenyl)-2H-[1,2,4] triazole-3-yl]-piperidin-1-yl}-ethyl ketone (263)
1-{(R)-2-[3-(3-hydroxyl-phenyl)-[1,2,4] oxadiazole-5-yl]-piperidin-1-yl}-2-phenoxy group-ethyl ketone (264)
1-{(R)-2-[3-(4-hydroxyl-phenyl)-[1,2,4] oxadiazole-5-yl]-piperidin-1-yl}-2-phenoxy group-ethyl ketone (265)
1-{(R)-2-[5-(2-fluoro-phenyl)-2H-[1,2,4] triazole-3-yl]-piperidin-1-yl}-2-phenoxy group-ethyl ketone (266)
1-{(R)-2-[5-(3,5-, two fluoro-phenyl)-2H-[1,2,4] triazole-3-yl]-piperidin-1-yl}-2-phenoxy group-ethyl ketone (267)
1-{(R)-2-[5-(3-fluoro-phenyl)-2H-[1,2,4] triazole-3-yl]-piperidin-1-yl}-2-phenoxy group-ethyl ketone (268)
1-{(R)-2-[5-(3,4-, two fluoro-phenyl)-2H-[1,2,4] triazole-3-yl]-piperidin-1-yl}-2-phenoxy group-ethyl ketone (269), and
2-phenoxy group-1-{(R)-2-[5-(2,4,5-, three fluoro-phenyl)-[1,2,4] oxadiazole-3-yl]-piperidin-1-yl )-ethyl ketone (270), and pharmaceutical salts and esters thereof.

In some embodiments, the mitochondrial lipid shunt modulator is a compound as disclosed in U.S. Pat. No. 7,799,933 (e.g., a compound of Formula (VI) or a salt or derivative thereof), incorporated herein by reference in its entirety. In some embodiments, the mitochondrial lipid shunt modulator is a compound of Formula (VI):

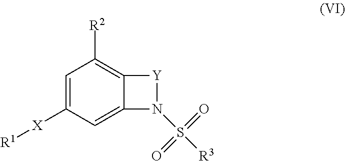

wherein X is —NHC(O)— or —C(O)NH—; Y is —C(R⁴R⁵)—, —C(R⁴R⁵)C(R⁶R⁷)—, C(R⁴R⁵)C(R⁶R⁷) C(R⁸R⁹)—, —C(R⁴R⁵)C(R⁶R⁷)C(R⁸R⁹)C(R¹⁰R¹¹)— or —CR⁴═CR⁶—; R¹ is aryl or heteroaryl, which aryl or heteroaryl is substituted with —C(R¹²R¹³)[C(R¹⁴R¹⁵)]ₙC(O)OR¹⁶, and which aryl or heteroaryl in addition is optionally substituted with 1 to 2 substitutents independently selected from the group consisting of lower-alkyl, hydroxy, halogen, lower-alkoxy, fluoro-lower-alkyl and fluoro-lower-alkoxy; R² is hydrogen, lower-alkyl, hydroxy, halogen, lower-alkoxy, fluoro-lower-alkyl or fluoro-lower-alkoxy; R³ is aryl which is optionally substituted with 1 to 3 substituents independently selected form the group consisting of halogen, cyano, hydroxy, lower-alkyl, fluoro-lower-alkyl, lower-alkoxy, fluoro-lower-alkoxy, lower-alkyl-C(O), lower-alkyl-C(O)—NH, lower-alkyl-C(O)—N(lower-alkyl), lower-alkyl-S(O)₂, NH₂—S(O)₂, N(H,lower-alkyl)-S(O)₂, N(lower-alkyl)₂-S(O)₂, NH₂—C(O), N (H, lower-alkyl)-C(O), N(lower-alkyl)₂-C(O) and lower-alkoxy-C(O), wherein lower-alkyl is optionally substituted with hydroxy, lower-alkoxy, NH₂, N(H,lower-alkyl) or N(lower-alkyl)₂; R⁴, RV, R⁶, R⁷, R⁸, R⁹, R¹⁰ and R¹¹ independently from each other are hydrogen, halogen, lower-alkyl, lower-alkoxy, fluoro-lower-alkyl, fluoro-lower-alkoxy, hydroxy or hydroxy-lower-alkyl; R¹², R³, R¹⁴ and R¹⁵ independently from each other are hydrogen, halogen, lower-alkyl, lower-alkoxy, fluoro-lower-alkyl, fluoro-lower-alkoxy, hydroxy, hydroxy-lower-alkyl; or R³ is H and R¹² is —(CH₂)₁₋₃— and forms a bridge to the aryl or heteroaryl, to which the —C(R¹²R¹³)[C(R¹⁴R¹⁵)]ₙC(O)OR¹⁶ is bound; R¹⁶ is hydrogen or lower-alkyl; n is 0 or 1; and pharmaceutically acceptable salts and esters thereof..

In some embodiments, the mitochondrial lipid shunt modulator of Formula (VI) is a CPT1 inhibitor. In some embodiments, the mitochondrial lipid shunt modulator of Formula (VI) is selected from the group consisting of:
(4-{[1-(5-chloro-2-methoxyl group-benzenesulfonyl)-1H-indoles-6-carbonyl]-amino}-phenyl)-acetate (271)
(4-{[1-(2-methoxyl group-5-methyl-benzenesulfonyl)-1H-indoles-6-carbonyl]-amino}-phenyl)-acetate (272)
(4-{[1-(3,5-dimethyl-benzenesulfonyl)-1H-indoles-6-carbonyl]-amino}-phenyl)-acetate (273)
(4-{[1-(3-chloro-benzenesulfonyl)-1H-indoles-6-carbonyl]-amino}-phenyl)-acetate (274)
(4-{[1-(2-methoxyl group-5-methyl-benzenesulfonyl)-2,3-dihydro-1H-indoles-6-carbonyl]-amino}-phenyl)-acetate (275)
(4-{[1-(3,5-dimethyl-benzenesulfonyl)-2,3-dihydro-1H-indoles-6-carbonyl]-amino}-phenyl)-acetate (276)
(4-{[1-(3-chloro-benzenesulfonyl)-2,3-dihydro-1H-indoles-6-carbonyl]-amino}-phenyl)-acetate (277)
(2-{[1-(3,5-dimethyl-benzenesulfonyl)-2,3-dihydro-1H-indoles-6-carbonyl]-amino}-thiazole-4-yl)-acetate (278)
(2-{[1-(3-chloro-benzenesulfonyl)-2,3-dihydro-1H-indoles-6-carbonyl]-amino}-thiazole-4-yl)-acetate (279)
(4-{[1-(5-chloro-2-methoxyl group-benzenesulfonyl)-2,3-dihydro-1H-indoles-6-carbonyl]-amino}-phenyl)-acetate (280)
(2-{[1-(5-chloro-2-methoxyl group-benzenesulfonyl)-2,3-dihydro-1H-indoles-6-carbonyl]-amino}-thiazole-4-yl)-acetate (281)
(2-{[1-(5-chloro-2-methoxyl group-benzenesulfonyl)-2,3-dihydro-1H-indoles-6-carbonyl]-amino}-5-methyl-thiazole-4-yl)-acetate (281)
(3-{[1-(5-chloro-2-methoxyl group-benzenesulfonyl)-2,3-dihydro-1H-indoles-6-carbonyl]-amino}-pyrazol-1-yl)-acetate (282)
(4-{[1-(5-chloro-2-methoxyl group-benzenesulfonyl)-4-methyl-2,3-dihydro-1H-indoles-6-carbonyl]-amino}-phenyl)-acetate (283)
(2-{[1-(5-chloro-2-methoxyl group-benzenesulfonyl)-4-methyl-2,3-dihydro-1H-indoles-6-carbonyl]-amino}-thiazole-4-yl)-acetate (284)
(2-{[1-(5-chloro-2-methoxyl group-benzenesulfonyl)-2,3,4,5-tetrahydrochysene-1H-benzo [b] azepine-8-carbonyl]-amino}-thiazole-4-yl)-acetate (285)
(4-{[1-(2-methoxyl group-5-methyl-benzenesulfonyl)-1,2,3,4-tetrahydrochysene-quinoline-7-carbonyl]-amino}-phenyl)-acetate (286)
(5-{[1-(2-methoxyl group-5-methyl-benzenesulfonyl)-1,2,3,4-tetrahydrochysene-quinoline-7-carbonyl]-amino}-[1,2,4] thiadiazoles-3-yl)-acetate (287) hydroxyl-(4-{[1-(2-methoxyl group-5-methyl-benzenesulfonyl)-1,2,3,4-tetrahydrochysene-quinoline-7-carbonyl]-amino}-phenyl)-acetate (288)
2-{[1-(2-methoxyl group-5-methyl-benzenesulfonyl)-1,2,3,4-tetrahydrochysene-quinoline-7-carbonyl]-amino}-5,6-dihydro-4H-cyclopenta thiazole-4-carboxylic acid (289)
(2-{[1-(2-methoxyl group-5-methyl-benzenesulfonyl)-1,2,3,4-tetrahydrochysene-quinoline-7-carbonyl]-amino}-thiazole-4-yl)-acetate (290)
2-(4-{[1-(2-methoxyl group-5-methyl-benzenesulfonyl)-1,2,3,4-tetrahydrochysene-quinoline-7-carbonyl]-amino}-phenyl)-propionic acid (291)
2-{[1-(2-methoxyl group-5-methyl-benzenesulfonyl)-1,2,3,4-tetrahydrochysene-quinoline-7-carbonyl]-amino-4,5,6,7-tetrahydrochysene-benzothiazole-4-carboxylic acid (292)
4-[1-(5-chloro-2-methoxyl group-benzenesulfonyl)-1,2,3,4-tetrahydrochysene-quinoline-7-base formamyl]-phenyl}-acetate (293)
(2-{[1-(3,5-dimethyl-benzenesulfonyl)-2,3-dihydro-1H-indoles-6-carbonyl]-amino}-thiazole-4-yl)-acetate (294)
(2-{[1-(3-chloro-benzenesulfonyl)-2,3-dihydro-1H-indoles-6-carbonyl]-amino}-thiazole-4-yl)-acetate (295)
(4-{[1-(5-chloro-2-methoxyl group-benzenesulfonyl)-2,3-dihydro-1H-indoles-6-carbonyl]-amino}-phenyl)-acetate (296)
(2-{[1-(5-chloro-2-methoxyl group-benzenesulfonyl)-2,3-dihydro-1H-indoles-6-carbonyl]-amino}-thiazole-4-yl)-acetate (297)
(3-{[1-(5-chloro-2-methoxyl group-benzenesulfonyl)-2,3-dihydro-1H-indoles-6-carbonyl]-amino}-pyrazol-1-yl)-acetate (298)
(4-{[1-(5-chloro-2-methoxyl group-benzenesulfonyl)-4-methyl-2,3-dihydro-1H-indoles-6-carbonyl]-amino}-phenyl)-acetate (299)
(2-{[1-(5-chloro-2-methoxyl group-benzenesulfonyl)-4-methyl-2,3-dihydro-1H-indoles-6-carbonyl]-amino}-thiazole-4-yl)-acetate (300)
(4-{[1-(2-methoxyl group-5-methyl-benzenesulfonyl)-1,2,3,4-tetrahydrochysene-quinoline-7-carbonyl]-amino}-phenyl)-acetate (301)
(2-{[1-(2-methoxyl group-5-methyl-benzenesulfonyl)-1,2,3,4-tetrahydrochysene-quinoline-7-carbonyl]-amino}-thiazole-4-yl)-acetate (302)
   and pharmaceutical salts and esters thereof.

In some embodiments, the mitochondrial lipid shunt modulator is a compound as disclosed in U.S. Pat. No. 8,697,677 (e.g., perhexiline or a salt or derivative thereof), incorporated herein by reference in its entirety. In some embodiments, the mitochondrial lipid shunt modulator is a compound of Formula (VII):

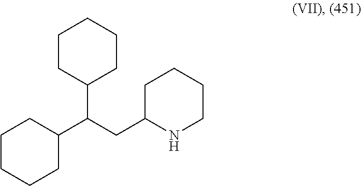 [SMILES: C1CCC(C(CC2CCCCN2)C2CCCCC2)CC1]

In some embodiments, the mitochondrial lipid shunt modulator is a compound as disclosed in Kennedy et al (Biochem Pharmacol (1996) 52(2):273-280, e.g., amiodarone or a salt or derivative thereof), incorporated herein by reference in its entirety. In some embodiments, the mitochondrial lipid shunt modulator is a compound of Formula (VIII):

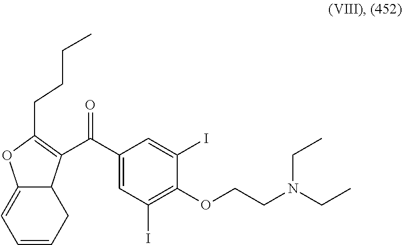 [SMILES: CCCCC1=C(C(=O)c2cc(I)c(OCCN(CC)CC)c(I)c2)C2CC=CC=C2O1]

In some embodiments, the mitochondrial lipid shunt modulator is a compound as disclosed in US Patent Publication No. 2011/0048980 (e.g., etomoxir or a salt or derivative thereof), incorporated herein by reference in its entirety. In some embodiments, mitochondrial lipid shunt modulator is a compound of Formula (IX):

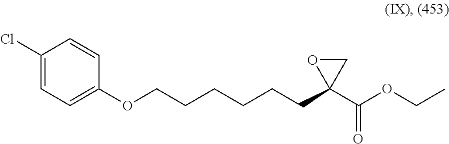 [SMILES: CCOC(=O)[C@@]1(CCCCCCOc2ccc(Cl)cc2)CO1]

In some embodiments, the mitochondrial lipid shunt modulator is a compound as disclosed in Sepa-Kishiet et al (Am J Physiol Reg Integr Comp Physiol (2016) 311:R77-R787, e.g., oxfenicine or a salt or derivative thereof), incorporated herein by reference in its entirety. In some embodiments, the mitochondrial lipid shunt modulator is a compound of Formula (X):

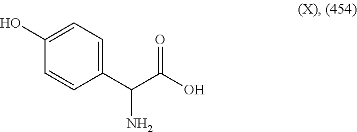 [SMILES: NC(C(=O)O)c1ccc(O)cc1]

In some embodiments, the mitochondrial lipid shunt modulator is a compound as disclosed in U.S. Patent Publication No. 2010/0210695 (e.g., a compound of Formula (I), Formula (II), Formula (III), Formula (IV), or a salt or derivative thereof), incorporated herein by reference in its entirety. In some embodiments, the mitochondrial lipid shunt modulator is a compound of Formula (XI):

X+—CH₂—CH(Z)—CH₂—Y— (XI)

wherein: X⁺ is N⁺(R₁, R₂, R₃) or P⁺(R₁, R₂, R₃); (R₁, R₂, R₃), being the same or different, are selected from the group consisting of hydrogen, a C₁-C₉ straight or branched alkyl group, —CH═NH(NH₂), —NH₂, and —OH; or one or more of R₁, R₂ and R₃, together with the nitrogen atom to which they are linked, form a saturated or unsaturated, monocyclic or bicyclic heterocyclic system; with the proviso that at least one of the R₁, R₂ and R₃ is different from hydrogen; Z is selected from —OR₄, —OCOOR₄, —OCONH₄, —OCSNHR₄, —OCSOR₄, —NHR₄, —NHCOR₄, —NHCSR₄, —NHCOOR₄, —NHCSOR₄, —NHCONHR₄, —NHCSNHR₄, —NHSOR₄, —NHSONHR₄, —NHSO₂R₄, —NHSO₂NHR₄, and —SR₄, —R₄ is a C₁-C₂₀ saturated or unsaturated, straight or branched alkyl group, optionally substituted with an A₁ group, A₁ is selected from the group consisting of a halogen atom, or an aryl, heteroaryl, aryloxy or heteroaryloxy group, said aryl, heteroaryl, aryloxy or heteroaryloxy groups being optionally substituted with one or more C₁-C₂₀ saturated or unsaturated, straight or branched alkyl or alkoxy group and/or halogen atom; Y⁻ is selected from the group consisting of —COO⁻, PO₃H⁻, —OPO₃H⁻, and tetrazolate-5-yl their (R,S) racemic mixtures, their single R or S enantiomers, their pharmaceutically acceptable salts and prodrugs thereof.

In some embodiments, the mitochondrial lipid shunt modulator is R-4-trimethylammonium-3-[tetradecylcarbamoyl)-aminobutyrate (ST1326) (455).

In some embodiments, the mitochondrial lipid shunt modulator disclosed in U.S. Patent Publication No. 2010/0210695 is a compound of Formula (XII):

(CH₃)₃N⁺OCH₂CH(ZR)CH₂COO— (XII),

wherein: Z=ureido, carbamate, sulfonamide, or sulfamide moieties; and R═C₇ to C₁₄ linear alkyl chains, their (R,S) racemic mixtures, their single R or S enantiomers, their pharmaceutically acceptable salts and prodrugs thereof.

In some embodiments, the mitochondrial lipid shunt modulator disclosed in U.S. Patent Publication No. 2010/0210695 is a compound of Formula (XIII):

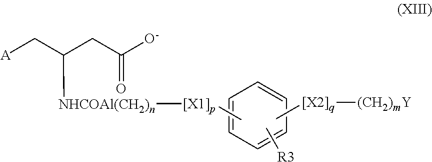

wherein A is selected between —N⁺(RR₁R₂), —P⁺(RR₁R₂), in which R, R₁, R₂ are the same or different and are selected from the group consisting of (C₁-C₂) alkyl, phenyl and phenyl-(C₁-C₂) alkyl; Al is O or NH or is absent; n is an integer number ranging from 0 to 20; p is 0 or 1: q is 0, 1; X1 is O or S; X2 is O or S; m is an integer number ranging from 1 to 20; Y selected among H, phenyl and phenoxy; R3 is selected among H, halogen, linear or branched (C₁-C₄) alkyl and (C₁-C₄) alkoxy, their (R,S) racemic mixtures, their single R or S enantiomers, their pharmaceutically acceptable salts and prodrugs thereof.

In some embodiments, the mitochondrial lipid shunt modulator disclosed in U.S. Patent Publication No. 2010/0210695 is a compound of Formula (XIV):

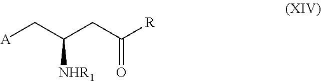

wherein A is selected among —N(R₂R₃), —N(R₂R₃R₄) and —C(R₂R₃R₄), in which the same or different R₂, R₃, R₄ are selected among H, alkyl C₁-C₂, phenyl, phenyl-alkyl C₁-C₂; R is selected among —OH, —O^{θ}, linear or branched alkoxy C₁-C₄, optionally replaced by a carboxy or alkoxycarbonyl group C₁-C₄, or the group Y—Z, in which: Y═—O—(CH₂)ₙ—O—, —O—(CH₂)ₙ—NH—, —S—(CH₂)ₙ—O—, —S—(CH₂)ₙ—NH—, where n is selected among 1, 2 and 3, or —O—(CH₂)ₙ—NH—, where n is selected among 0, 1, 2 and 3; and

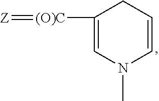

R₁ is selected among —COOR₅, —CONHR₅, —SOR₅, —SONHR₅, —SO₂R₅ and —SO₂NHR₅, in which R₅ is a saturated or unsaturated, linear of branched alkyl C₁-C₂₀, replaced by aryl C₆-C₁₀, aryloxy heteroaryl C₄-C₁₀ containing 1 or more atoms selected among N, O and S, heteroaryloxy C₄-C₁₀ containing 1 or more atoms selected among N, O and S, in turn replaced by saturated or unsaturated, linear or branched alkyl or alkoxy C₁-C₂₀; their (R,S) racemic mixtures, their single R or S enantiomers, their pharmaceutically acceptable salts and prodrugs thereof.

In some embodiments, the mitochondrial lipid shunt modulator is a compound disclosed in U.S. Patent Publication No. 2016/0271137 (e.g., a compound of Formula (VI), (VIIa), (VIIb), (VIII), (IX), or a salt or derivative thereof), incorporated herein by reference in its entirety. In some embodiments, the mitochondrial lipid shunt modulator is a compound of Formula (XV):

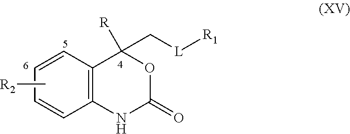

wherein L is selected from carbamate, urea, or amide including, for example,

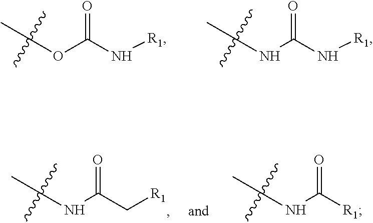

and wherein R is selected from halo; CF₃; cyclopropyl; optionally substituted C₁₋₅ alkyl, wherein the C₁₋₅ alkyl is substituted with halo, oxo, —OH, —CN, —NH₂, CO₂H, and C₁₋₃ alkoxy; wherein R₁ is selected from substituted phenyl where the substituents are selected from F, CF₃, Me, OMe, or isopropyl; wherein R₂ is Cl, Ph, 1-(2-pyridone), 4-isoxazol, 3-pyrazol, 4-pyrazol, 1-pyrazol, 5-(1,2,4-triazol), 1-(1,2,4-triazol), 2-imidazolo, 1-(2-pyrrolidone), 3-(1,3-oxazolidin-2-one); and wherein the chiral center at C4 is racemic, (S), (R), or any ratio of enantiomers.

In some embodiments, the mitochondrial lipid shunt modulator is a compound of Formula (XVI-a) or (XVI-b):

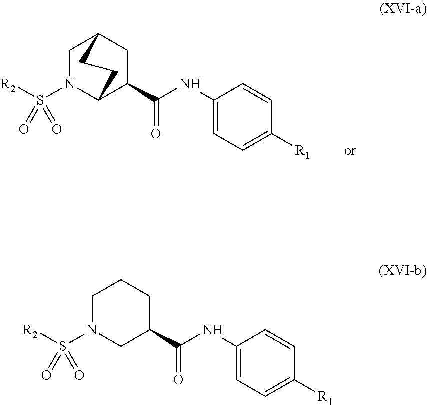

wherein R₁ is selected from OMe, OiPr, OCF₃, OPh, CH₂Ph, F, CH₃, CF₃, and benzyl; and wherein R₂ is selected from C₁₋₄ alkyl; phenyl; substituted phenyl where substitutents are selected from OMe, CF₃, F, tBu, iPr and thio; 2-pyridine; 3-pyridine; and N-methy imidazole.

In some embodiments, the mitochondrial lipid shunt modulator is a compound of Formula (XVII):

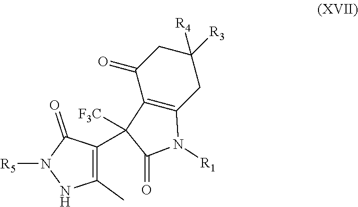

wherein R₁ is selected from H, unsubstituted phenyl; substituted phenyl where substituents are selected from F, Me, Et, Cl, OMe, OCF₃, and CF₃; C₁₋₆ alkyl; and C₃₋₆ cycloalkyl; wherein R₃ and R₄ are independently selected from H; C₁₋₃ alkyl; and phenyl; or R₃ and R₄ taken together form a cycloalkyl of formula —(CH₂)ₙ— where n=2, 3, 4 and 5; wherein R₅ is selected from methyl; CF₃; cyclopropyl; unsubstituted phenyl; mono- and disubstituted phenyl where substitutents are selected from F, Me, Et, CN, iPr, Cl, OMe, OPh, OCF₃, and CF₃; unsubstituted heteroaromatic groups; and imidazole.

In some embodiments, the mitochondrial lipid shunt modulator is a compound of Formula (XVIII):

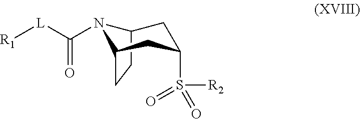

wherein L is selected from urea, amide,

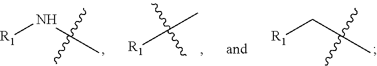

wherein R₁ is selected form 2-, 3-, and 4-pyridine; pyrimidine; unsubstituted heteroaryls such as isoxazol, pyrazol, triazol, imidazole; and unsubstituted phenyl; ortho, meta or para-substituted phenyl where substitutents are F, Me, Et, Cl, OMe, OCF₃, and CF₃, Cl, iPr and phenyl; and wherein R₂ is selected from Cl; iPr; phenyl; ortho, meta or para-substituted phenyl where substitutents are F, Me, Et, C, OMe, OCF₃, and CF₃; and heteroaryls such as 2-, 3-, and 4-pyridine, pyrimidine, and isoxazole, pyrazole, triazole, and imidazole.

In some embodiments, the mitochondrial lipid shunt modulator is a compound of selected from:

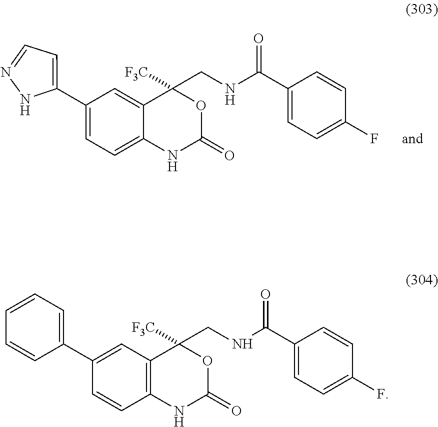 [SMILES: O=C1Nc2ccc(-c3ccn[nH]3)cc2[C@](CNC(=O)c2ccc(F)cc2)(C(F)(F)F)O1 ; O=C1Nc2ccc(-c3ccccc3)cc2[C@](CNC(=O)c2ccc(F)cc2)(C(F)(F)F)O1]

and pharmaceutically acceptable salts and esters thereof.

In some embodiments, the mitochondrial lipid shunt modulator is a compound selected from:

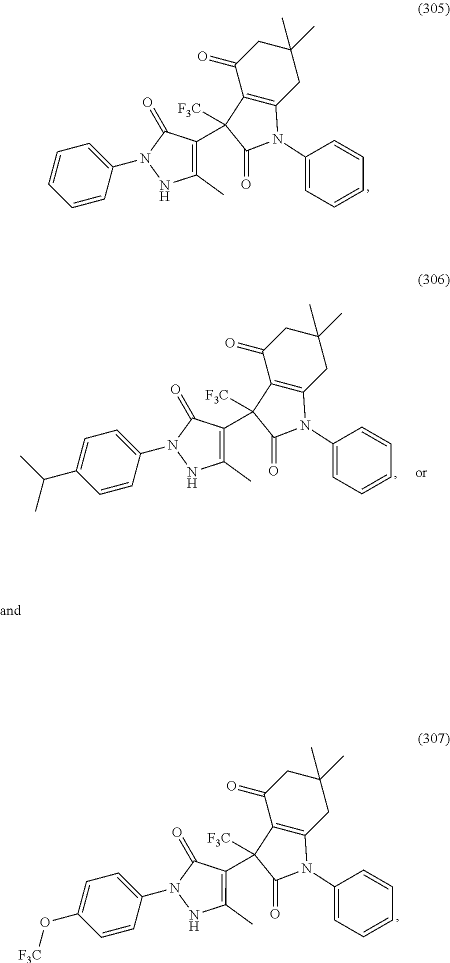 [SMILES: Cc1[nH]n(-c2ccccc2)c(=O)c1C1(C(F)(F)F)C(=O)N(c2ccccc2)C2=C1C(=O)CC(C)(C)C2 ; Cc1[nH]n(-c2ccc(C(C)C)cc2)c(=O)c1C1(C(F)(F)F)C(=O)N(c2ccccc2)C2=C1C(=O)CC(C)(C)C2 ; Cc1[nH]n(-c2ccc(OC(F)(F)F)cc2)c(=O)c1C1(C(F)(F)F)C(=O)N(c2ccccc2)C2=C1C(=O)CC(C)(C)C2]

and pharmaceutically acceptable salts and esters thereof.

In some embodiments, the mitochondrial lipid shunt modulator is a compound selected from:

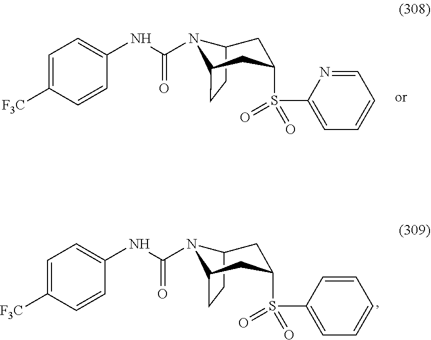

and pharmaceutically acceptable salts and esters thereof.

In some embodiments, the mitochondrial lipid shunt modulator is a compound selected from:

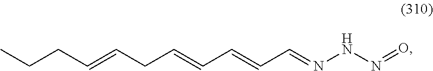

and pharmaceutically acceptable salts and esters thereof.

In some embodiments, the mitochondrial lipid shunt modulator is a compound selected from:

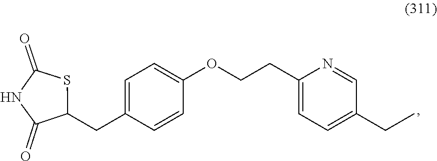 [SMILES: CCc1ccc(CCOc2ccc(CC3SC(=O)NC3=O)cc2)nc1]

and pharmaceutically acceptable salts and esters thereof.

In some embodiments, the mitochondrial lipid shunt modulator is a compound disclosed in Bordet et al (Pharmaceuticals (2010) 3(2):345-368, e.g., olesoxime, or a salt or derivative thereof), incorporated herein by reference in its entirety. In some embodiments, the mitochondrial lipid shunt modulator is:

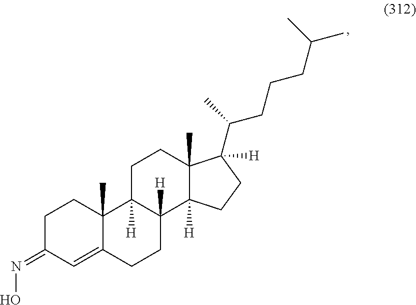

and pharmaceutically acceptable salts and esters thereof.

In some embodiments, the mitochondrial lipid shunt modulator is a compound as disclosed in Dolma et al (Cancer Cell (2003) 3(3):285-296, e.g., erastin, or a salt or derivative thereof), incorporated herein by reference in its entirety. In some embodiments, the mitochondrial lipid shunt modulator is:

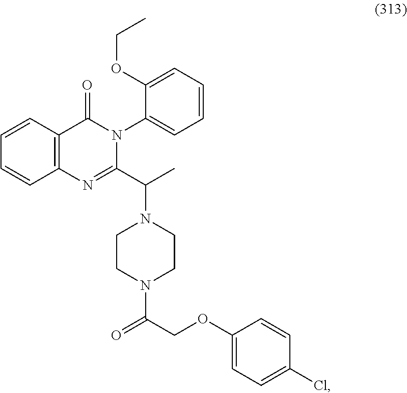 [SMILES: CCOc1ccccc1-n1c(C(C)N2CCN(C(=O)COc3ccc(Cl)cc3)CC2)nc2ccccc2c1=O]

and pharmaceutically acceptable salts and esters thereof.

In some embodiments, the mitochondrial lipid shunt modulator is a compound as disclosed in WO 2006/081337, incorporated herein by reference in its entirety. In some embodiments, the mitochondrial lipid shunt modulator is selected from:

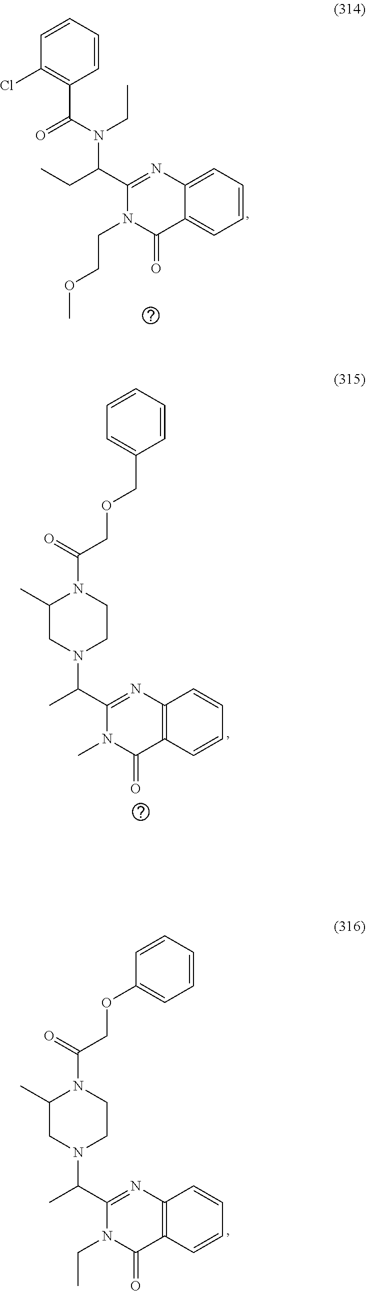 [SMILES: CCC(c1nc2ccccc2c(=O)n1CCOC)N(CC)C(=O)c1ccccc1Cl ; CC(c1nc2ccccc2c(=O)n1C)N1CCN(C(=O)COCc2ccccc2)C(C)C1 ; CCn1c(C(C)N2CCN(C(=O)COc3ccccc3)C(C)C2)nc2ccccc2c1=O]

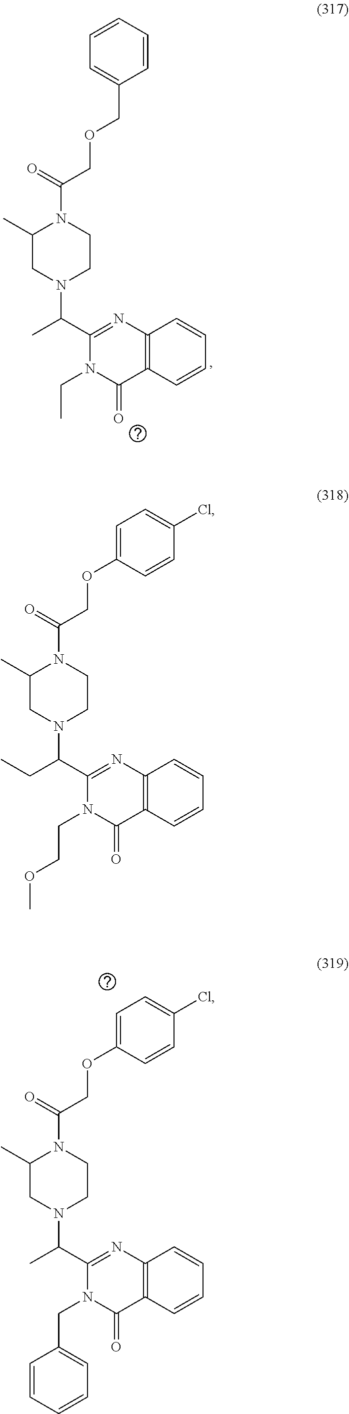 [SMILES: CCn1c(C(C)N2CCN(C(=O)COCc3ccccc3)C(C)C2)nc2ccccc2c1=O ; CCC(c1nc2ccccc2c(=O)n1CCOC)N1CCN(C(=O)COc2ccc(Cl)cc2)C(C)C1 ; CC(c1nc2ccccc2c(=O)n1Cc1ccccc1)N1CCN(C(=O)COc2ccc(Cl)cc2)C(C)C1]

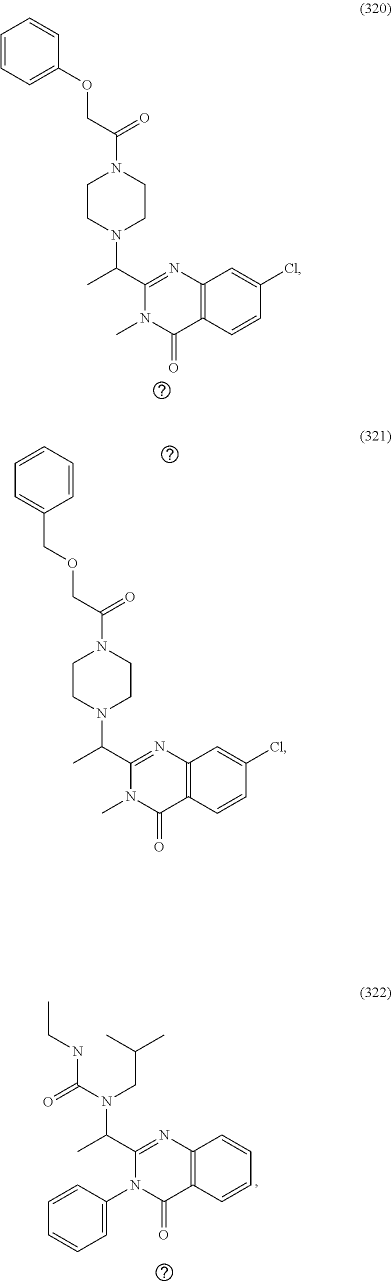 [SMILES: CC(c1nc2cc(Cl)ccc2c(=O)n1C)N1CCN(C(=O)COc2ccccc2)CC1 ; CC(c1nc2cc(Cl)ccc2c(=O)n1C)N1CCN(C(=O)COCc2ccccc2)CC1 ; CCNC(=O)N(CC(C)C)C(C)c1nc2ccccc2c(=O)n1-c1ccccc1]

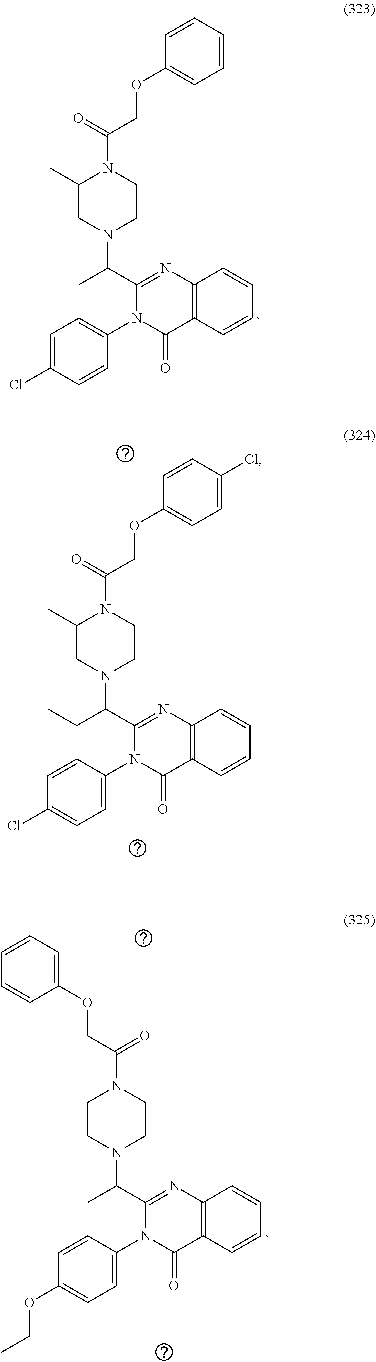 [SMILES: CC(c1nc2ccccc2c(=O)n1-c1ccc(Cl)cc1)N1CCN(C(=O)COc2ccccc2)C(C)C1 ; CCC(c1nc2ccccc2c(=O)n1-c1ccc(Cl)cc1)N1CCN(C(=O)COc2ccc(Cl)cc2)C(C)C1 ; CCOc1ccc(-n2c(C(C)N3CCN(C(=O)COc4ccccc4)CC3)nc3ccccc3c2=O)cc1]

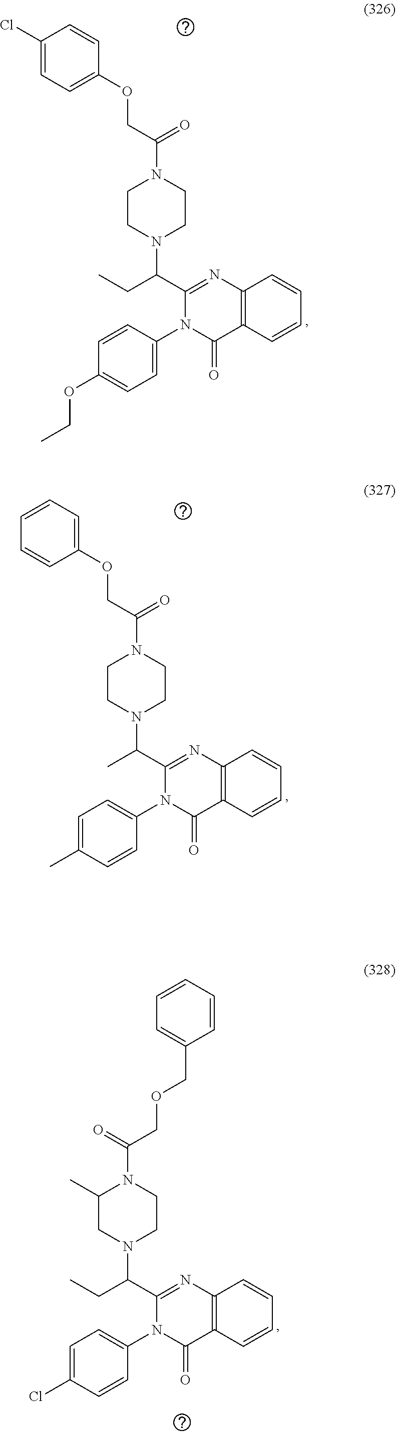 [SMILES: CCOc1ccc(-n2c(C(CC)N3CCN(C(=O)COc4ccc(Cl)cc4)CC3)nc3ccccc3c2=O)cc1 ; Cc1ccc(-n2c(C(C)N3CCN(C(=O)COc4ccccc4)CC3)nc3ccccc3c2=O)cc1 ; CCC(c1nc2ccccc2c(=O)n1-c1ccc(Cl)cc1)N1CCN(C(=O)COCc2ccccc2)C(C)C1]

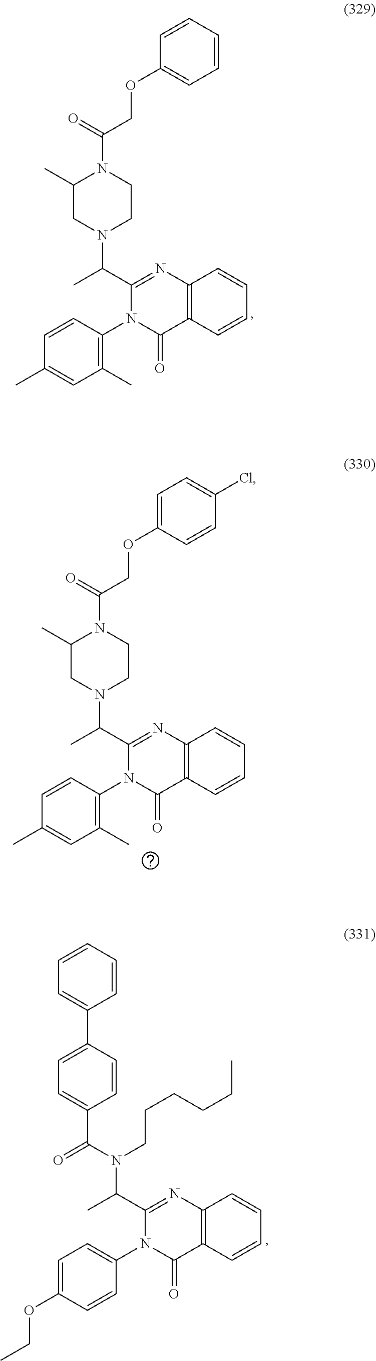 [SMILES: Cc1ccc(-n2c(C(C)N3CCN(C(=O)COc4ccccc4)C(C)C3)nc3ccccc3c2=O)c(C)c1 ; Cc1ccc(-n2c(C(C)N3CCN(C(=O)COc4ccc(Cl)cc4)C(C)C3)nc3ccccc3c2=O)c(C)c1 ; CCCCCCN(C(=O)c1ccc(-c2ccccc2)cc1)C(C)c1nc2ccccc2c(=O)n1-c1ccc(OCC)cc1]

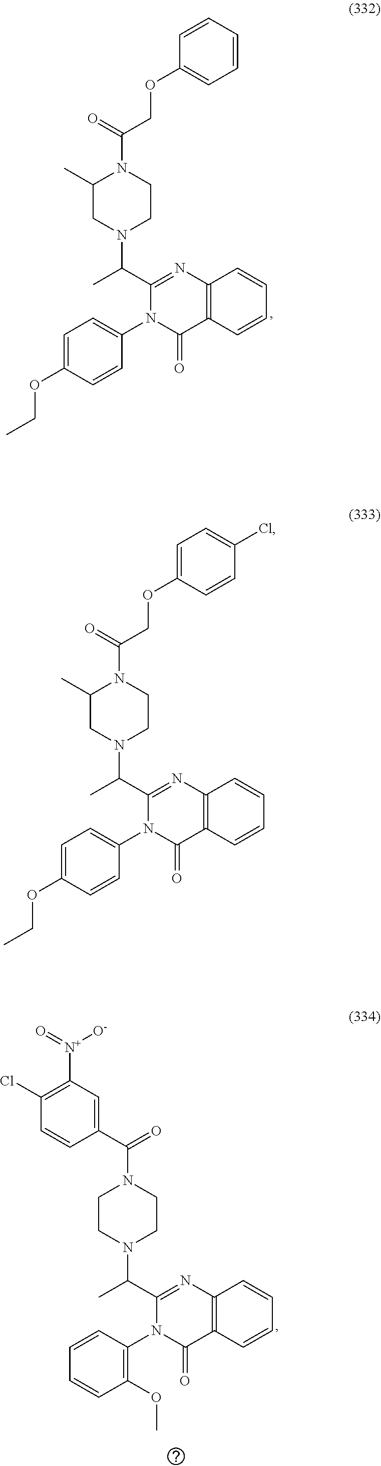 [SMILES: CCOc1ccc(-n2c(C(C)N3CCN(C(=O)COc4ccccc4)C(C)C3)nc3ccccc3c2=O)cc1 ; CCOc1ccc(-n2c(C(C)N3CCN(C(=O)COc4ccc(Cl)cc4)C(C)C3)nc3ccccc3c2=O)cc1 ; COc1ccccc1-n1c(C(C)N2CCN(C(=O)c3ccc(Cl)c([N+](=O)[O-])c3)CC2)nc2ccccc2c1=O]

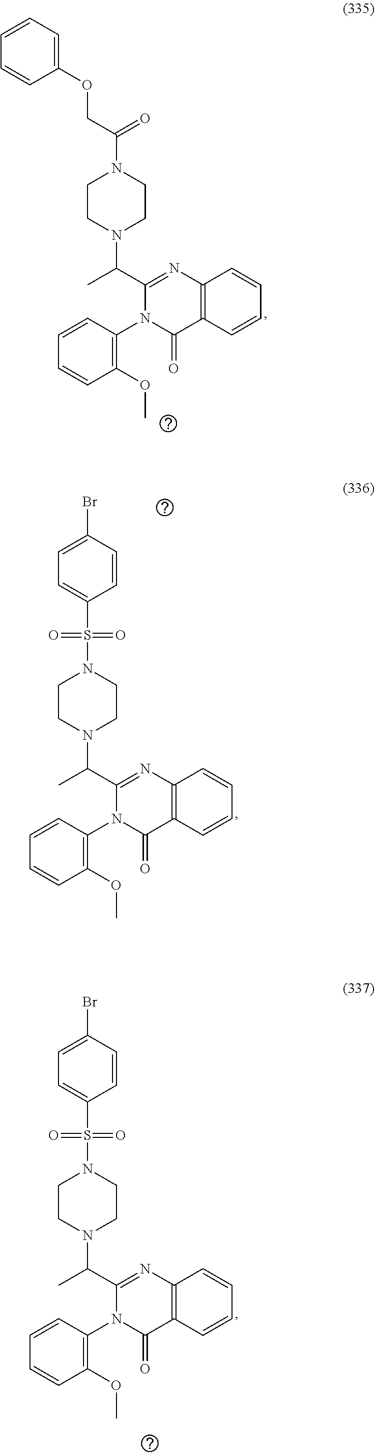 [SMILES: COc1ccccc1-n1c(C(C)N2CCN(C(=O)COc3ccccc3)CC2)nc2ccccc2c1=O ; COc1ccccc1-n1c(C(C)N2CCN(S(=O)(=O)c3ccc(Br)cc3)CC2)nc2ccccc2c1=O ; COc1ccccc1-n1c(C(C)N2CCN(S(=O)(=O)c3ccc(Br)cc3)CC2)nc2ccccc2c1=O]

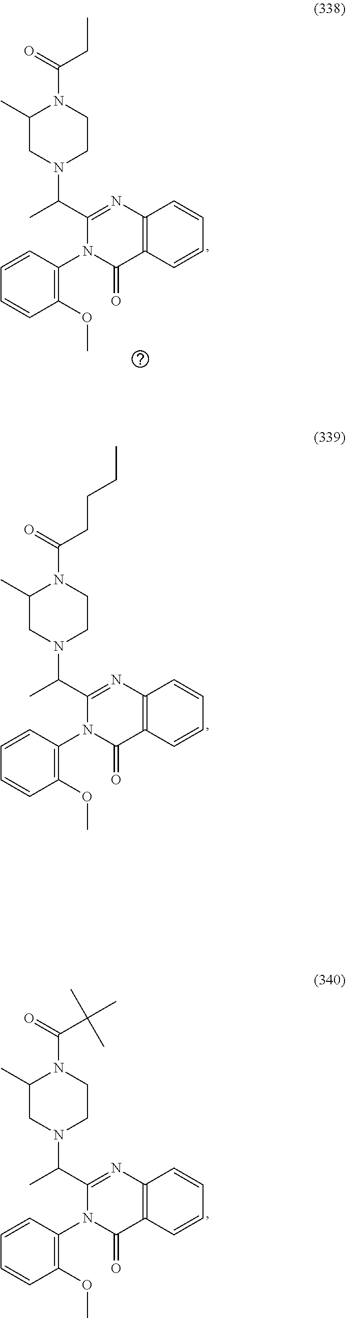 [SMILES: CCC(=O)N1CCN(C(C)c2nc3ccccc3c(=O)n2-c2ccccc2OC)CC1C ; CCCCC(=O)N1CCN(C(C)c2nc3ccccc3c(=O)n2-c2ccccc2OC)CC1C ; COc1ccccc1-n1c(C(C)N2CCN(C(=O)C(C)(C)C)C(C)C2)nc2ccccc2c1=O]

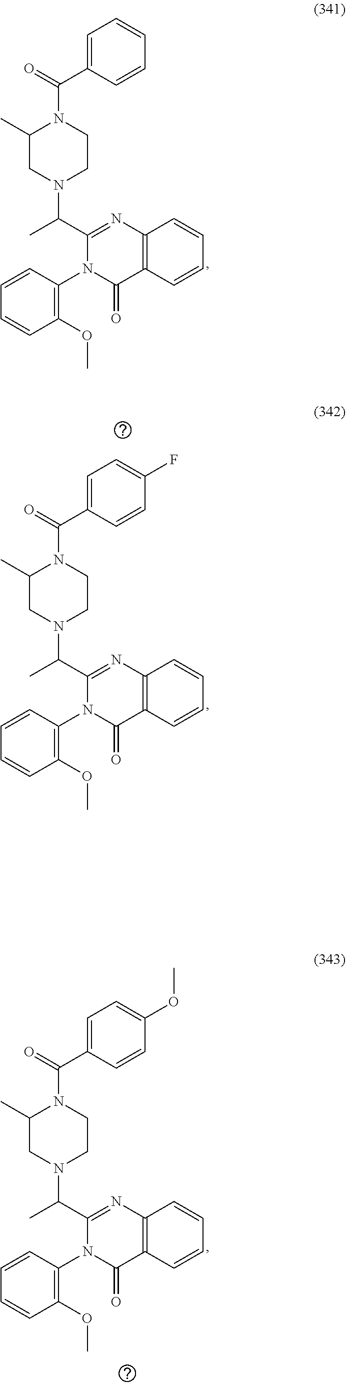 [SMILES: COc1ccccc1-n1c(C(C)N2CCN(C(=O)c3ccccc3)C(C)C2)nc2ccccc2c1=O ; COc1ccccc1-n1c(C(C)N2CCN(C(=O)c3ccc(F)cc3)C(C)C2)nc2ccccc2c1=O ; COc1ccc(C(=O)N2CCN(C(C)c3nc4ccccc4c(=O)n3-c3ccccc3OC)CC2C)cc1]

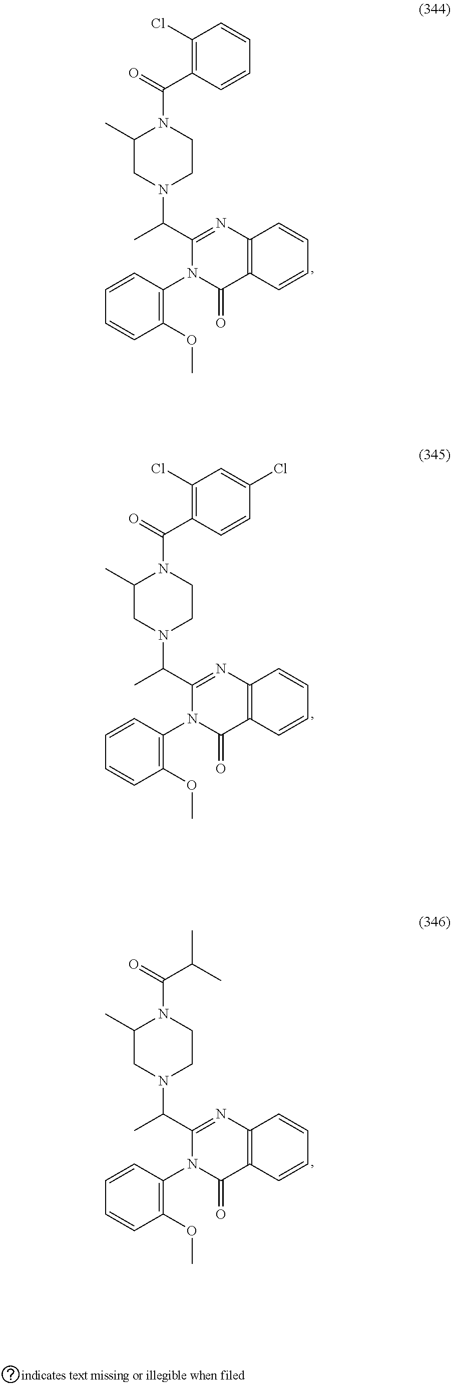 [SMILES: COc1ccccc1-n1c(C(C)N2CCN(C(=O)c3ccccc3Cl)C(C)C2)nc2ccccc2c1=O ; COc1ccccc1-n1c(C(C)N2CCN(C(=O)c3ccc(Cl)cc3Cl)C(C)C2)nc2ccccc2c1=O ; COc1ccccc1-n1c(C(C)N2CCN(C(=O)C(C)C)C(C)C2)nc2ccccc2c1=O]

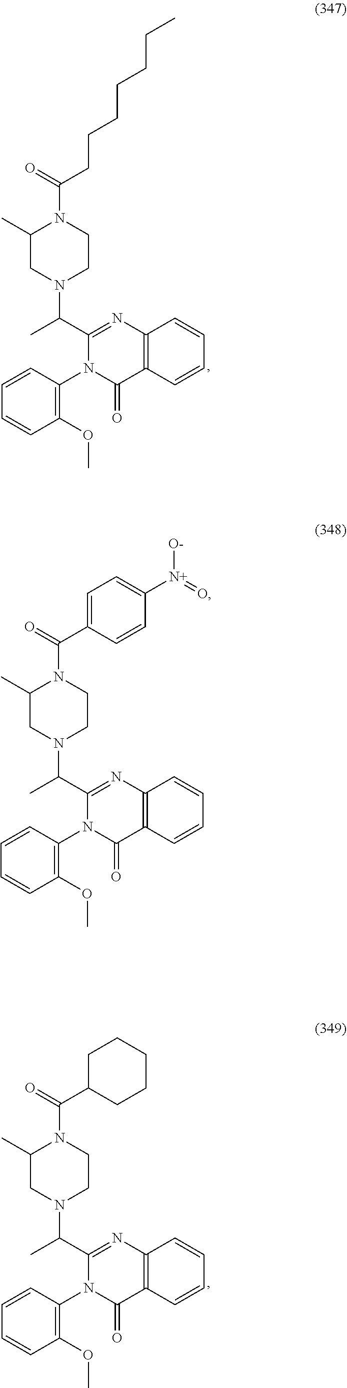 [SMILES: CCCCCCCC(=O)N1CCN(C(C)c2nc3ccccc3c(=O)n2-c2ccccc2OC)CC1C ; COc1ccccc1-n1c(C(C)N2CCN(C(=O)c3ccc([N+](=O)[O-])cc3)C(C)C2)nc2ccccc2c1=O ; COc1ccccc1-n1c(C(C)N2CCN(C(=O)C3CCCCC3)C(C)C2)nc2ccccc2c1=O]

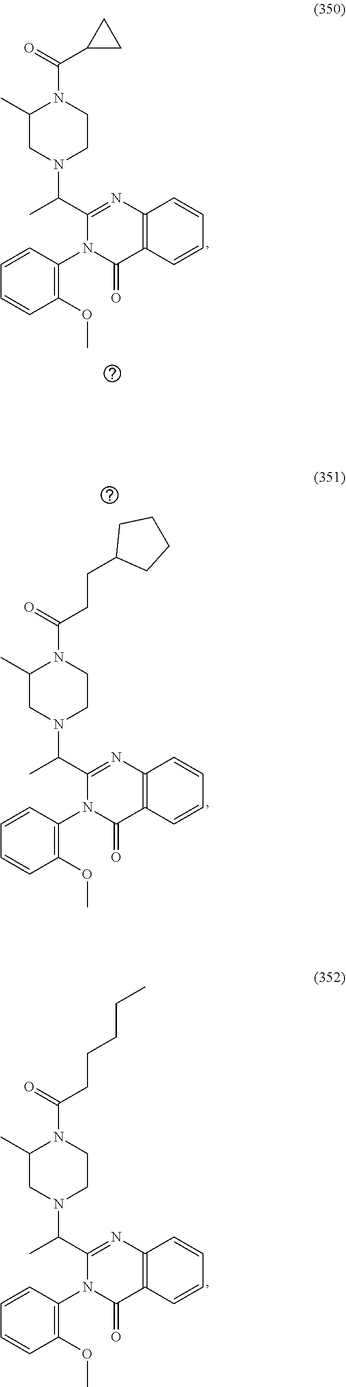 [SMILES: COc1ccccc1-n1c(C(C)N2CCN(C(=O)C3CC3)C(C)C2)nc2ccccc2c1=O ; COc1ccccc1-n1c(C(C)N2CCN(C(=O)CCC3CCCC3)C(C)C2)nc2ccccc2c1=O ; CCCCCC(=O)N1CCN(C(C)c2nc3ccccc3c(=O)n2-c2ccccc2OC)CC1C]

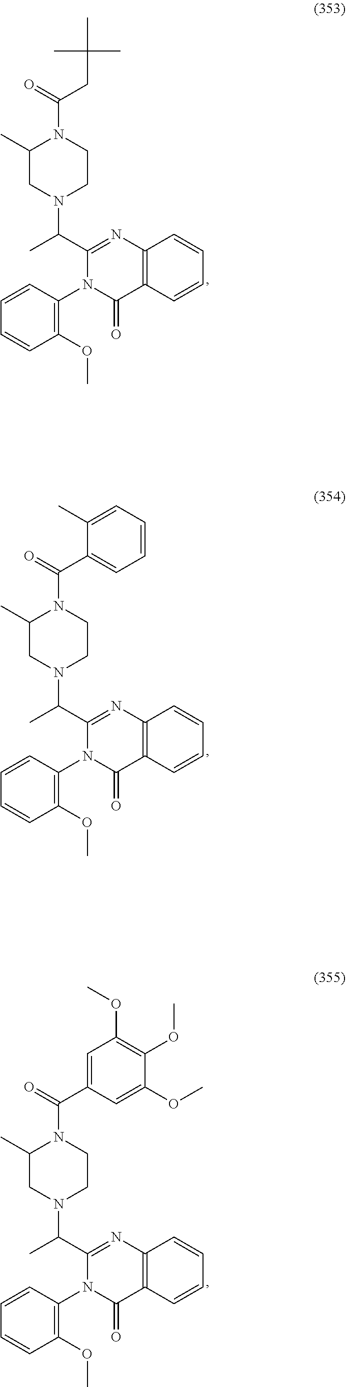 [SMILES: COc1ccccc1-n1c(C(C)N2CCN(C(=O)CC(C)(C)C)C(C)C2)nc2ccccc2c1=O ; COc1ccccc1-n1c(C(C)N2CCN(C(=O)c3ccccc3C)C(C)C2)nc2ccccc2c1=O ; COc1ccccc1-n1c(C(C)N2CCN(C(=O)c3cc(OC)c(OC)c(OC)c3)C(C)C2)nc2ccccc2c1=O]

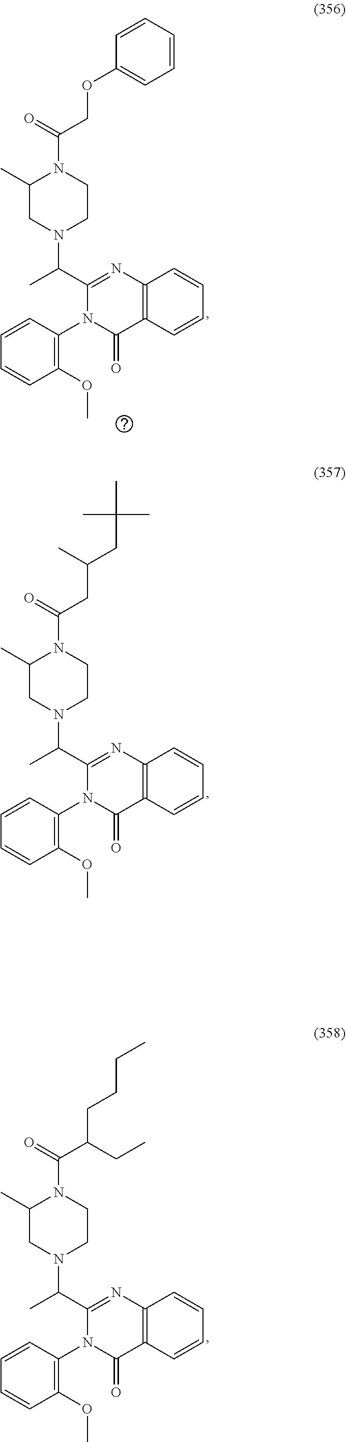 [SMILES: COc1ccccc1-n1c(C(C)N2CCN(C(=O)COc3ccccc3)C(C)C2)nc2ccccc2c1=O ; COc1ccccc1-n1c(C(C)N2CCN(C(=O)CC(C)CC(C)(C)C)C(C)C2)nc2ccccc2c1=O ; CCCCC(CC)C(=O)N1CCN(C(C)c2nc3ccccc3c(=O)n2-c2ccccc2OC)CC1C]

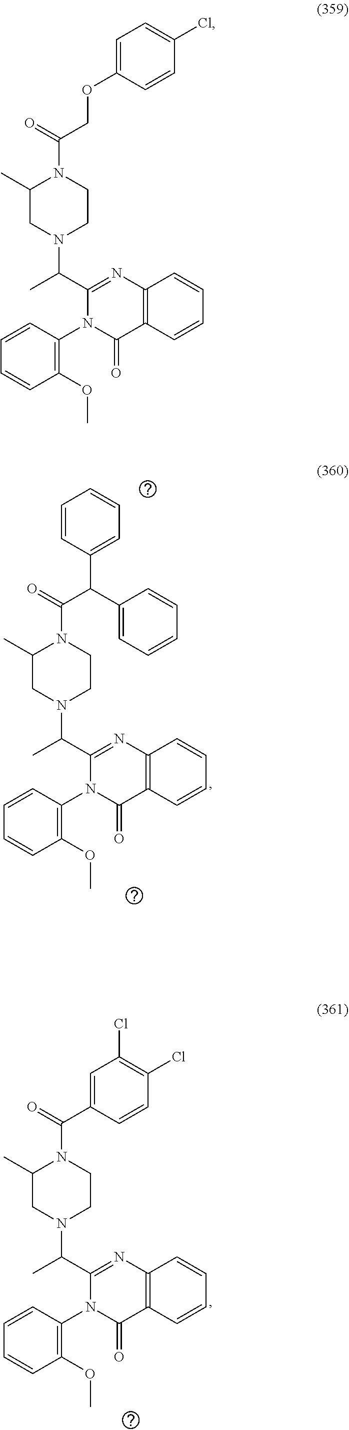 [SMILES: COc1ccccc1-n1c(C(C)N2CCN(C(=O)COc3ccc(Cl)cc3)C(C)C2)nc2ccccc2c1=O ; COc1ccccc1-n1c(C(C)N2CCN(C(=O)C(c3ccccc3)c3ccccc3)C(C)C2)nc2ccccc2c1=O ; COc1ccccc1-n1c(C(C)N2CCN(C(=O)c3ccc(Cl)c(Cl)c3)C(C)C2)nc2ccccc2c1=O]

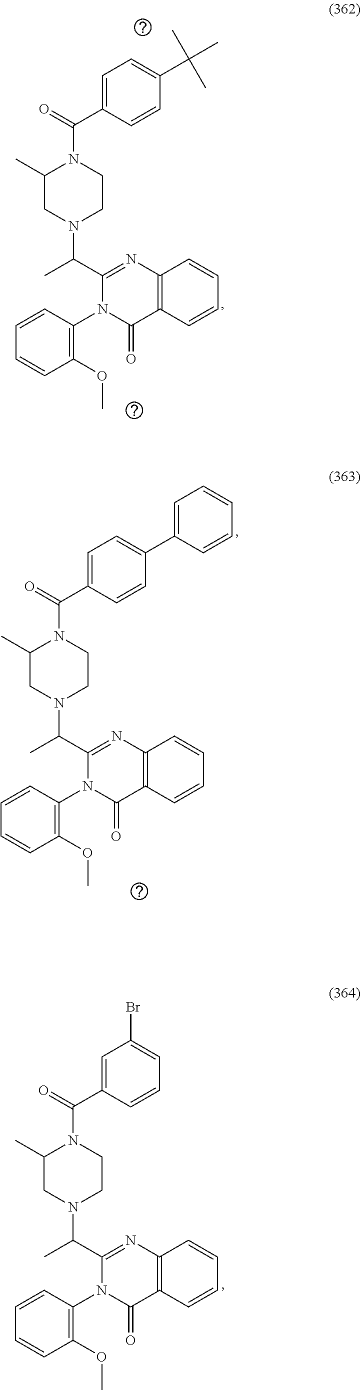 [SMILES: COc1ccccc1-n1c(C(C)N2CCN(C(=O)c3ccc(C(C)(C)C)cc3)C(C)C2)nc2ccccc2c1=O ; COc1ccccc1-n1c(C(C)N2CCN(C(=O)c3ccc(-c4ccccc4)cc3)C(C)C2)nc2ccccc2c1=O ; COc1ccccc1-n1c(C(C)N2CCN(C(=O)c3cccc(Br)c3)C(C)C2)nc2ccccc2c1=O]

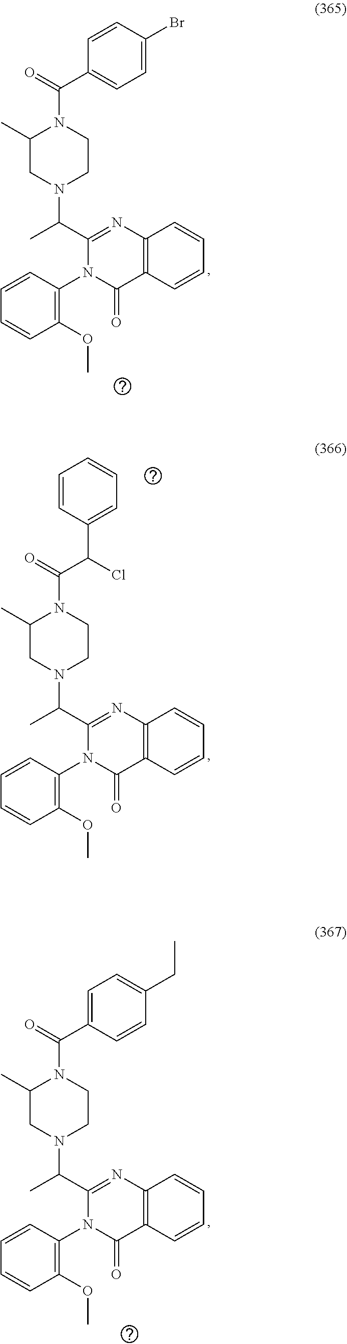 [SMILES: COc1ccccc1-n1c(C(C)N2CCN(C(=O)c3ccc(Br)cc3)C(C)C2)nc2ccccc2c1=O ; COc1ccccc1-n1c(C(C)N2CCN(C(=O)C(Cl)c3ccccc3)C(C)C2)nc2ccccc2c1=O ; CCc1ccc(C(=O)N2CCN(C(C)c3nc4ccccc4c(=O)n3-c3ccccc3OC)CC2C)cc1]

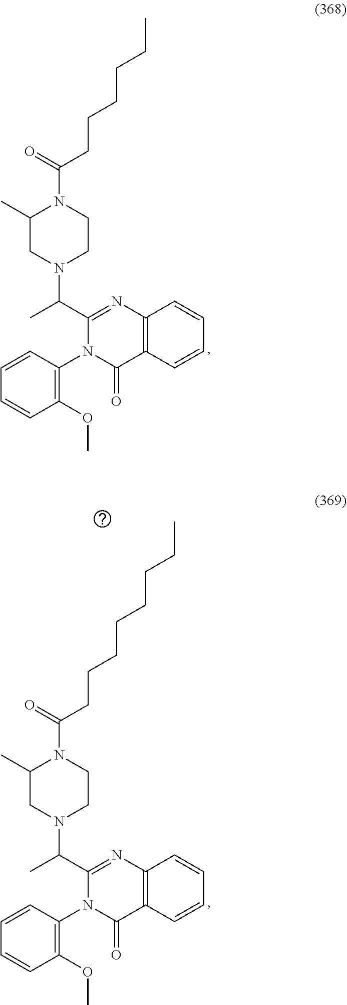 [SMILES: CCCCCCC(=O)N1CCN(C(C)c2nc3ccccc3c(=O)n2-c2ccccc2OC)CC1C ; CCCCCCCCC(=O)N1CCN(C(C)c2nc3ccccc3c(=O)n2-c2ccccc2OC)CC1C]

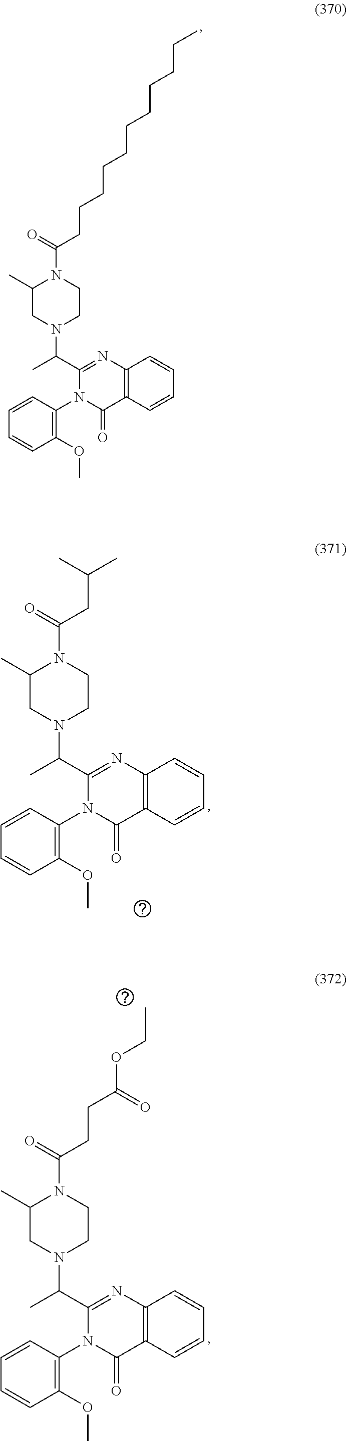 [SMILES: CCCCCCCCCCCC(=O)N1CCN(C(C)c2nc3ccccc3c(=O)n2-c2ccccc2OC)CC1C ; COc1ccccc1-n1c(C(C)N2CCN(C(=O)CC(C)C)C(C)C2)nc2ccccc2c1=O ; CCOC(=O)CCC(=O)N1CCN(C(C)c2nc3ccccc3c(=O)n2-c2ccccc2OC)CC1C]

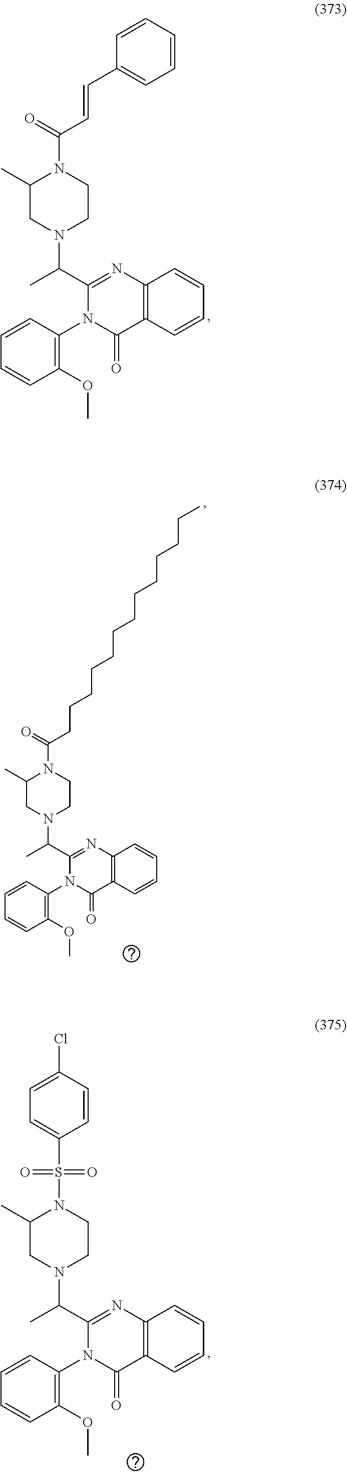 [SMILES: COc1ccccc1-n1c(C(C)N2CCN(C(=O)/C=C/c3ccccc3)C(C)C2)nc2ccccc2c1=O ; CCCCCCCCCCCCCC(=O)N1CCN(C(C)c2nc3ccccc3c(=O)n2-c2ccccc2OC)CC1C ; COc1ccccc1-n1c(C(C)N2CCN(S(=O)(=O)c3ccc(Cl)cc3)C(C)C2)nc2ccccc2c1=O]

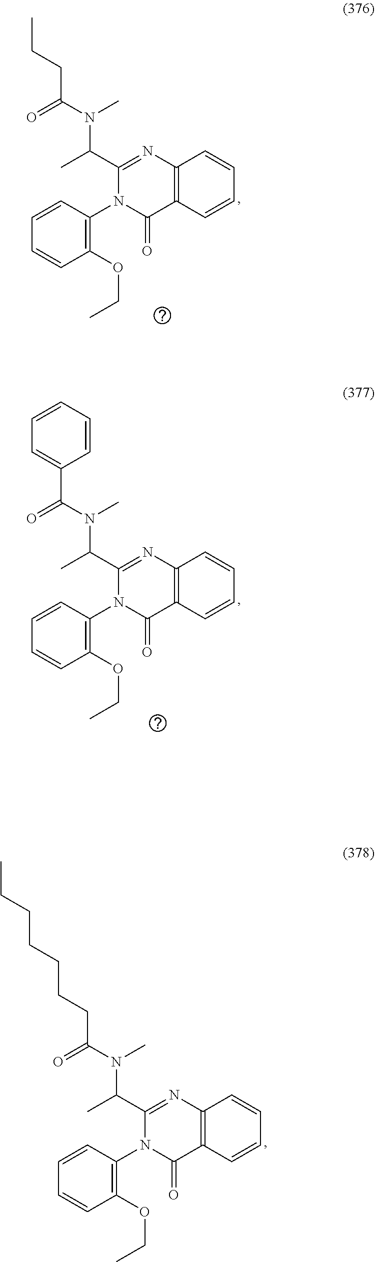 [SMILES: CCCC(=O)N(C)C(C)c1nc2ccccc2c(=O)n1-c1ccccc1OCC ; CCOc1ccccc1-n1c(C(C)N(C)C(=O)c2ccccc2)nc2ccccc2c1=O ; CCCCCCCC(=O)N(C)C(C)c1nc2ccccc2c(=O)n1-c1ccccc1OCC]

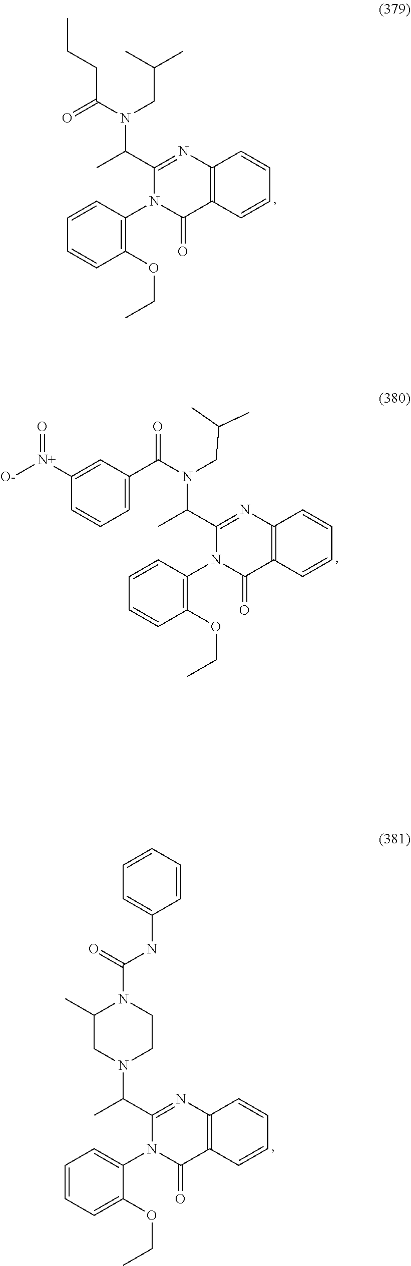 [SMILES: CCCC(=O)N(CC(C)C)C(C)c1nc2ccccc2c(=O)n1-c1ccccc1OCC ; CCOc1ccccc1-n1c(C(C)N(CC(C)C)C(=O)c2cccc([N+](=O)[O-])c2)nc2ccccc2c1=O ; CCOc1ccccc1-n1c(C(C)N2CCN(C(=O)Nc3ccccc3)C(C)C2)nc2ccccc2c1=O]

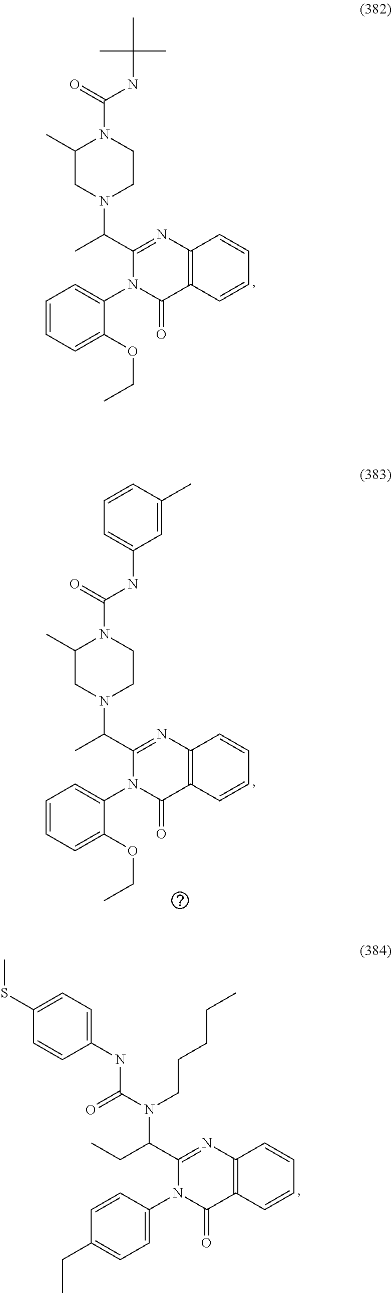 [SMILES: CCOc1ccccc1-n1c(C(C)N2CCN(C(=O)NC(C)(C)C)C(C)C2)nc2ccccc2c1=O ; CCOc1ccccc1-n1c(C(C)N2CCN(C(=O)Nc3cccc(C)c3)C(C)C2)nc2ccccc2c1=O ; CCCCCN(C(=O)Nc1ccc(SC)cc1)C(CC)c1nc2ccccc2c(=O)n1-c1ccc(CC)cc1]

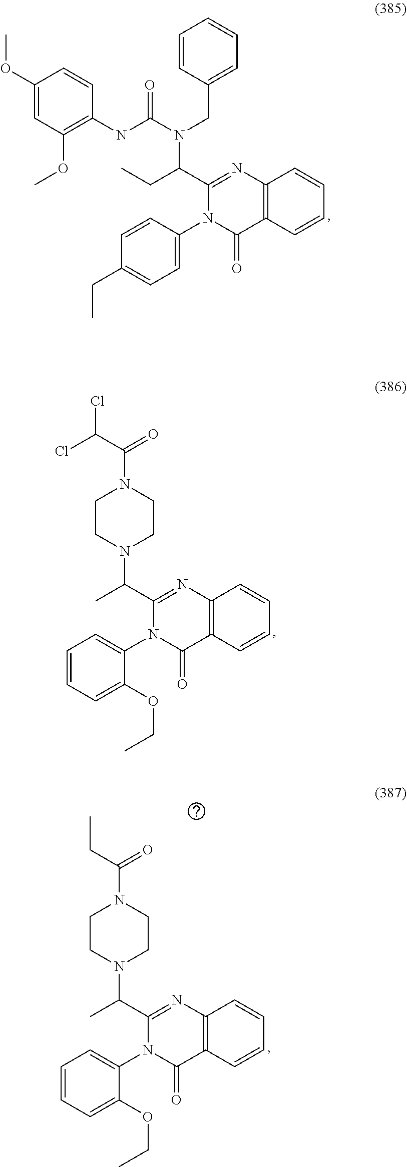 [SMILES: CCc1ccc(-n2c(C(CC)N(Cc3ccccc3)C(=O)Nc3ccc(OC)cc3OC)nc3ccccc3c2=O)cc1 ; CCOc1ccccc1-n1c(C(C)N2CCN(C(=O)C(Cl)Cl)CC2)nc2ccccc2c1=O ; CCOc1ccccc1-n1c(C(C)N2CCN(C(=O)CC)CC2)nc2ccccc2c1=O]

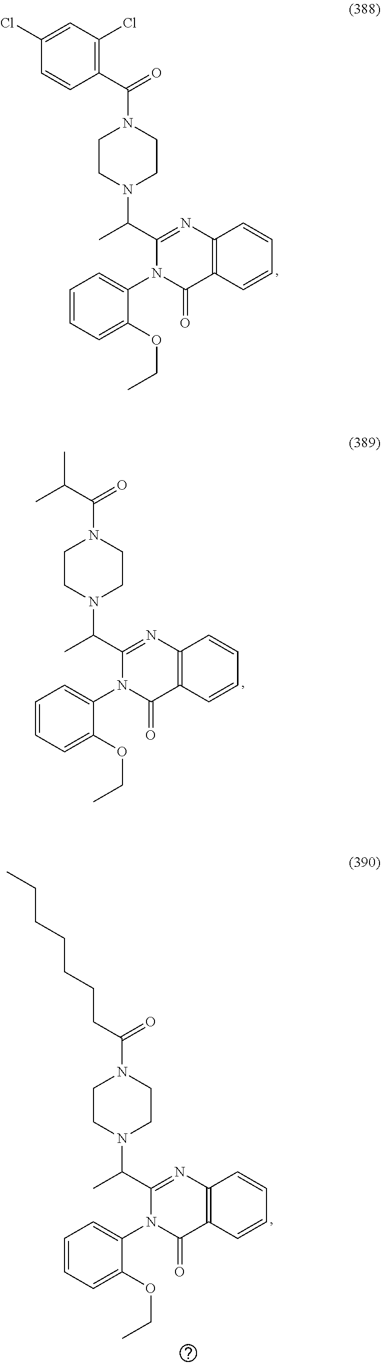 [SMILES: CCOc1ccccc1-n1c(C(C)N2CCN(C(=O)c3ccc(Cl)cc3Cl)CC2)nc2ccccc2c1=O ; CCOc1ccccc1-n1c(C(C)N2CCN(C(=O)C(C)C)CC2)nc2ccccc2c1=O ; CCCCCCCC(=O)N1CCN(C(C)c2nc3ccccc3c(=O)n2-c2ccccc2OCC)CC1]

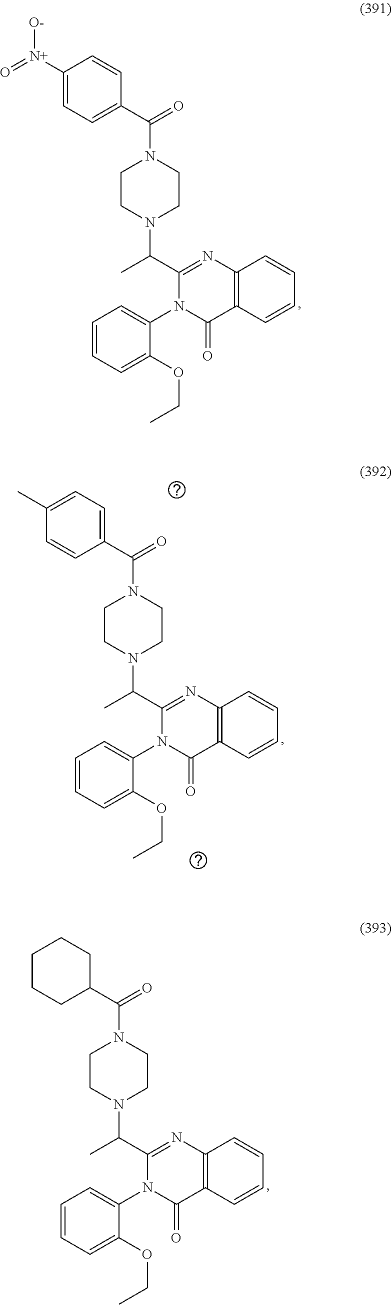 [SMILES: CCOc1ccccc1-n1c(C(C)N2CCN(C(=O)c3ccc([N+](=O)[O-])cc3)CC2)nc2ccccc2c1=O ; CCOc1ccccc1-n1c(C(C)N2CCN(C(=O)c3ccc(C)cc3)CC2)nc2ccccc2c1=O ; CCOc1ccccc1-n1c(C(C)N2CCN(C(=O)C3CCCCC3)CC2)nc2ccccc2c1=O]

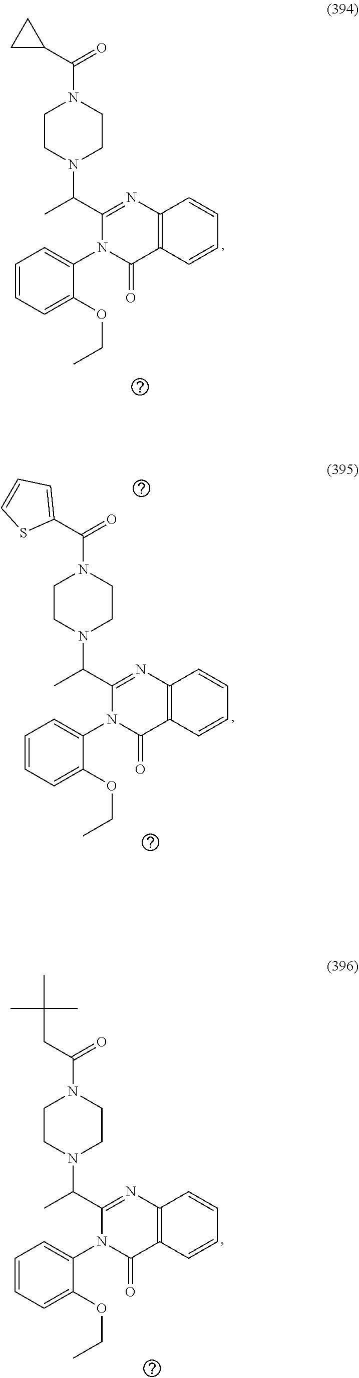 [SMILES: CCOc1ccccc1-n1c(C(C)N2CCN(C(=O)C3CC3)CC2)nc2ccccc2c1=O ; CCOc1ccccc1-n1c(C(C)N2CCN(C(=O)c3cccs3)CC2)nc2ccccc2c1=O ; CCOc1ccccc1-n1c(C(C)N2CCN(C(=O)CC(C)(C)C)CC2)nc2ccccc2c1=O]

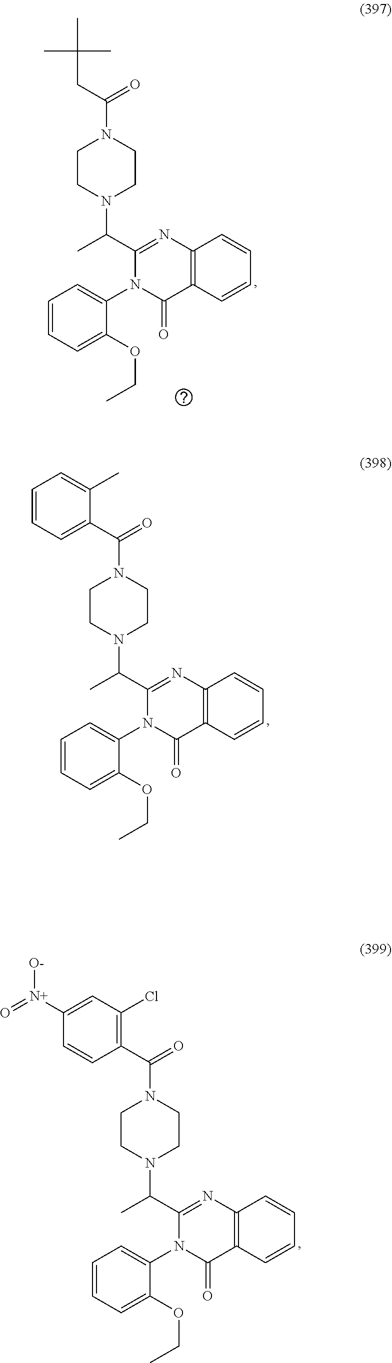 [SMILES: CCOc1ccccc1-n1c(C(C)N2CCN(C(=O)CC(C)(C)C)CC2)nc2ccccc2c1=O ; CCOc1ccccc1-n1c(C(C)N2CCN(C(=O)c3ccccc3C)CC2)nc2ccccc2c1=O ; CCOc1ccccc1-n1c(C(C)N2CCN(C(=O)c3ccc([N+](=O)[O-])cc3Cl)CC2)nc2ccccc2c1=O]

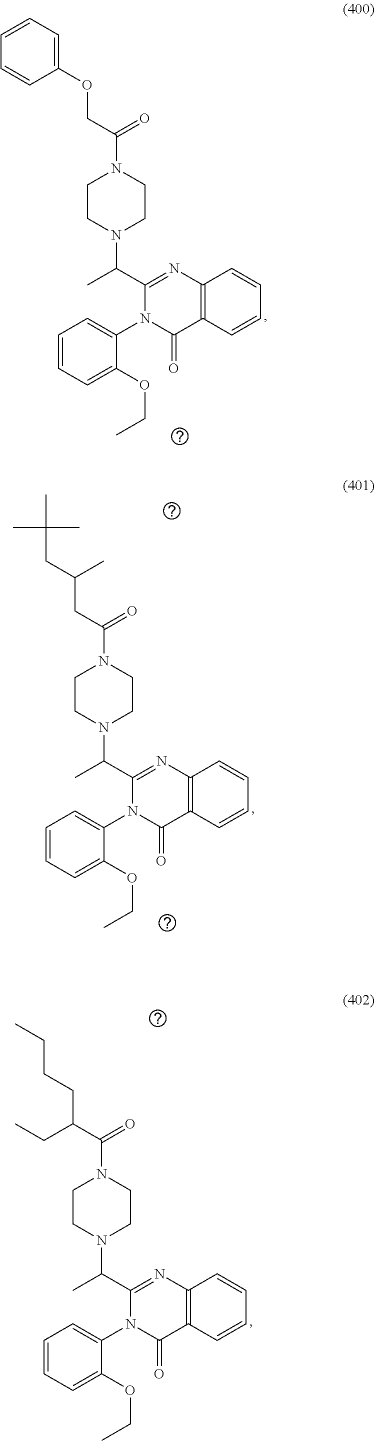 [SMILES: CCOc1ccccc1-n1c(C(C)N2CCN(C(=O)COc3ccccc3)CC2)nc2ccccc2c1=O ; CCOc1ccccc1-n1c(C(C)N2CCN(C(=O)CC(C)CC(C)(C)C)CC2)nc2ccccc2c1=O ; CCCCC(CC)C(=O)N1CCN(C(C)c2nc3ccccc3c(=O)n2-c2ccccc2OCC)CC1]

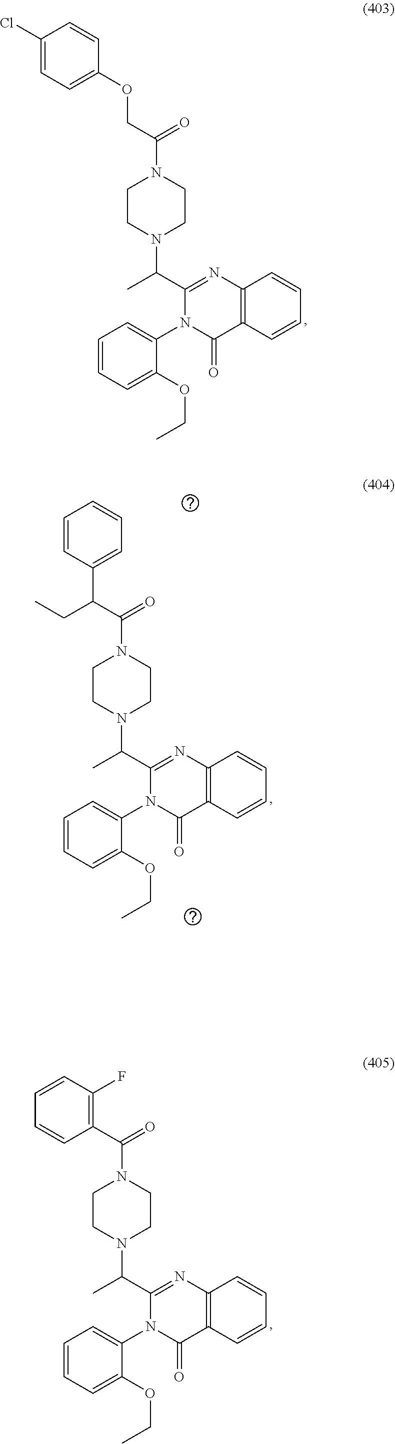 [SMILES: CCOc1ccccc1-n1c(C(C)N2CCN(C(=O)COc3ccc(Cl)cc3)CC2)nc2ccccc2c1=O ; CCOc1ccccc1-n1c(C(C)N2CCN(C(=O)C(CC)c3ccccc3)CC2)nc2ccccc2c1=O ; CCOc1ccccc1-n1c(C(C)N2CCN(C(=O)c3ccccc3F)CC2)nc2ccccc2c1=O]

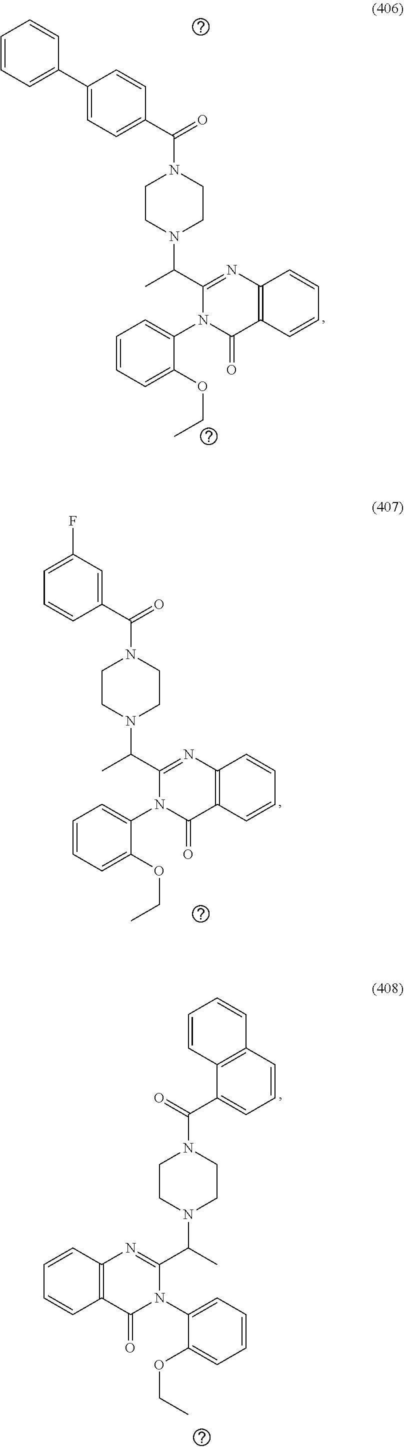 [SMILES: CCOc1ccccc1-n1c(C(C)N2CCN(C(=O)c3ccc(-c4ccccc4)cc3)CC2)nc2ccccc2c1=O ; CCOc1ccccc1-n1c(C(C)N2CCN(C(=O)c3cccc(F)c3)CC2)nc2ccccc2c1=O ; CCOc1ccccc1-n1c(C(C)N2CCN(C(=O)c3cccc4ccccc34)CC2)nc2ccccc2c1=O]

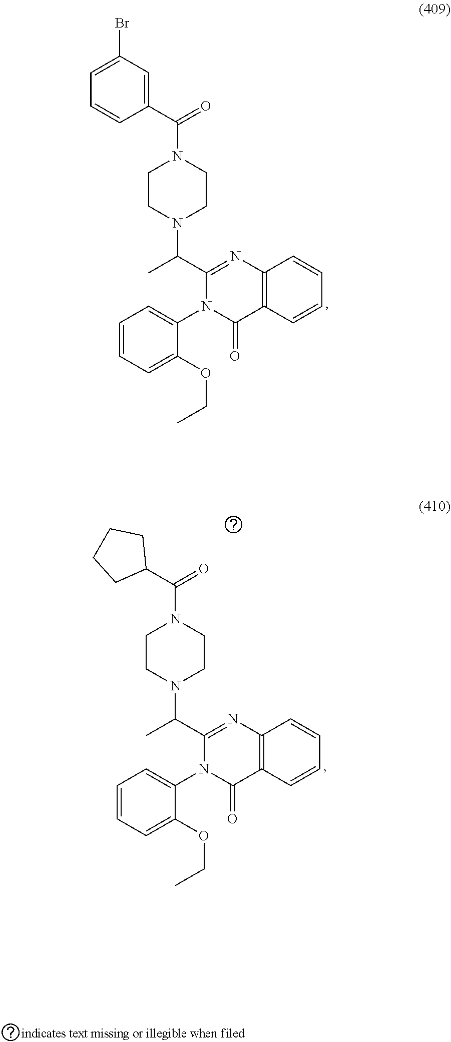 [SMILES: CCOc1ccccc1-n1c(C(C)N2CCN(C(=O)c3cccc(Br)c3)CC2)nc2ccccc2c1=O ; CCOc1ccccc1-n1c(C(C)N2CCN(C(=O)C3CCCC3)CC2)nc2ccccc2c1=O]

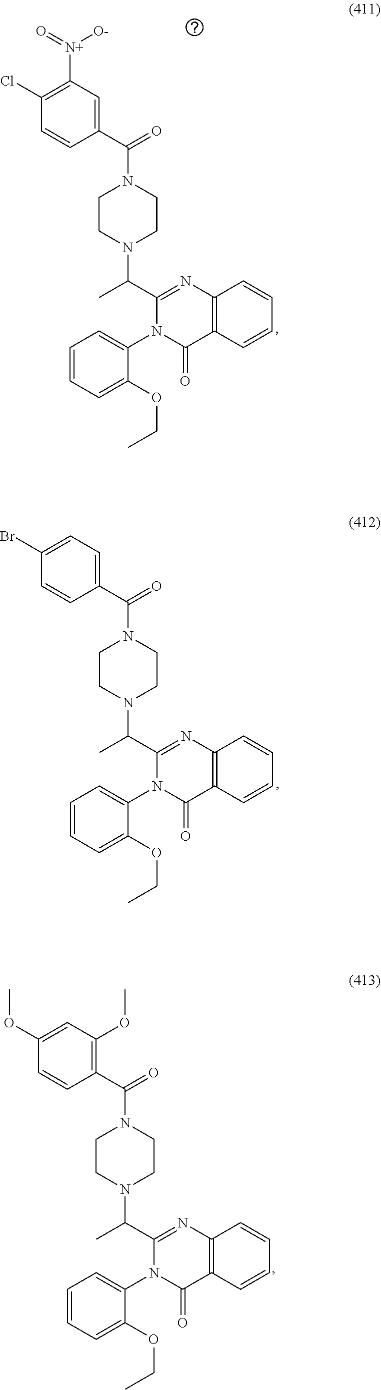 [SMILES: CCOc1ccccc1-n1c(C(C)N2CCN(C(=O)c3ccc(Cl)c([N+](=O)[O-])c3)CC2)nc2ccccc2c1=O ; CCOc1ccccc1-n1c(C(C)N2CCN(C(=O)c3ccc(Br)cc3)CC2)nc2ccccc2c1=O ; CCOc1ccccc1-n1c(C(C)N2CCN(C(=O)c3ccc(OC)cc3OC)CC2)nc2ccccc2c1=O]

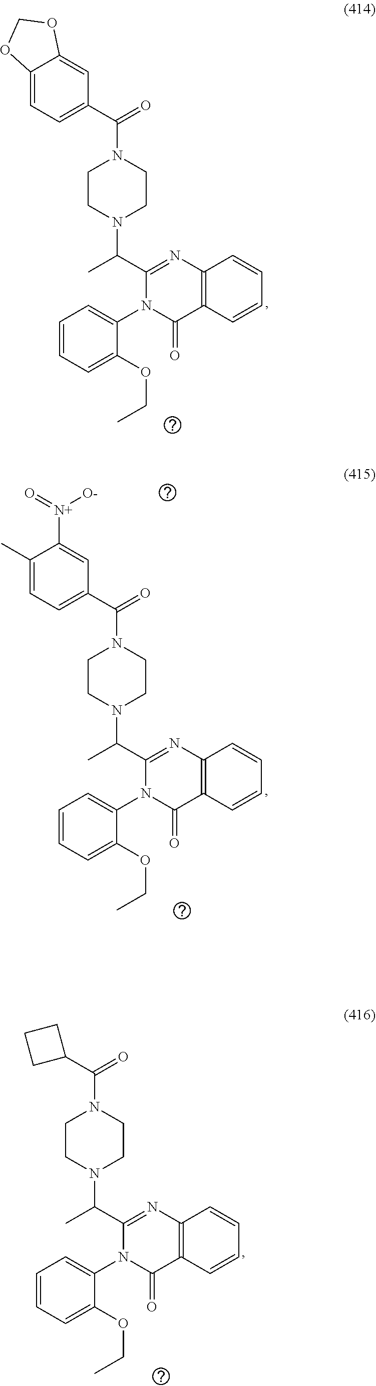 [SMILES: CCOc1ccccc1-n1c(C(C)N2CCN(C(=O)c3ccc4c(c3)OCO4)CC2)nc2ccccc2c1=O ; CCOc1ccccc1-n1c(C(C)N2CCN(C(=O)c3ccc(C)c([N+](=O)[O-])c3)CC2)nc2ccccc2c1=O ; CCOc1ccccc1-n1c(C(C)N2CCN(C(=O)C3CCC3)CC2)nc2ccccc2c1=O]

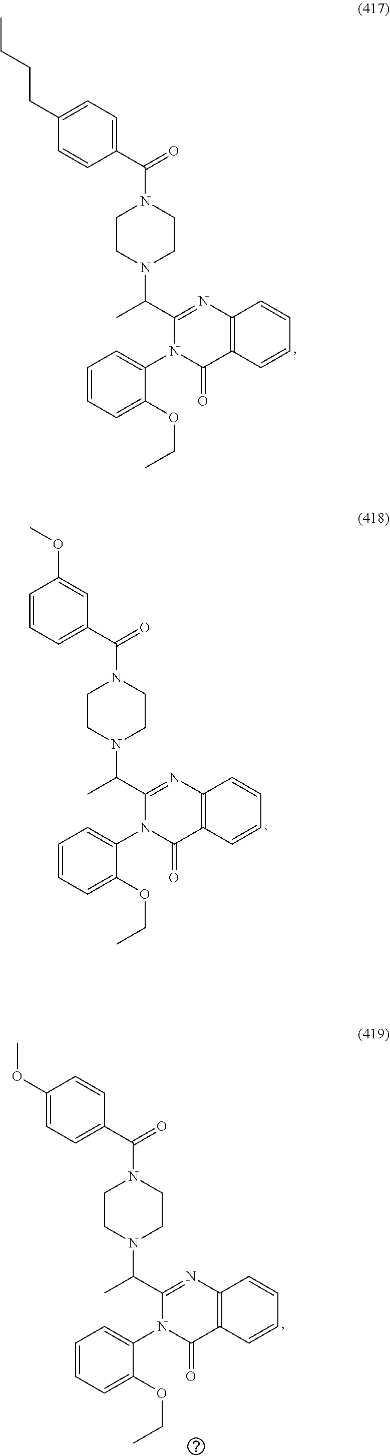 [SMILES: CCCCc1ccc(C(=O)N2CCN(C(C)c3nc4ccccc4c(=O)n3-c3ccccc3OCC)CC2)cc1 ; CCOc1ccccc1-n1c(C(C)N2CCN(C(=O)c3cccc(OC)c3)CC2)nc2ccccc2c1=O ; CCOc1ccccc1-n1c(C(C)N2CCN(C(=O)c3ccc(OC)cc3)CC2)nc2ccccc2c1=O]

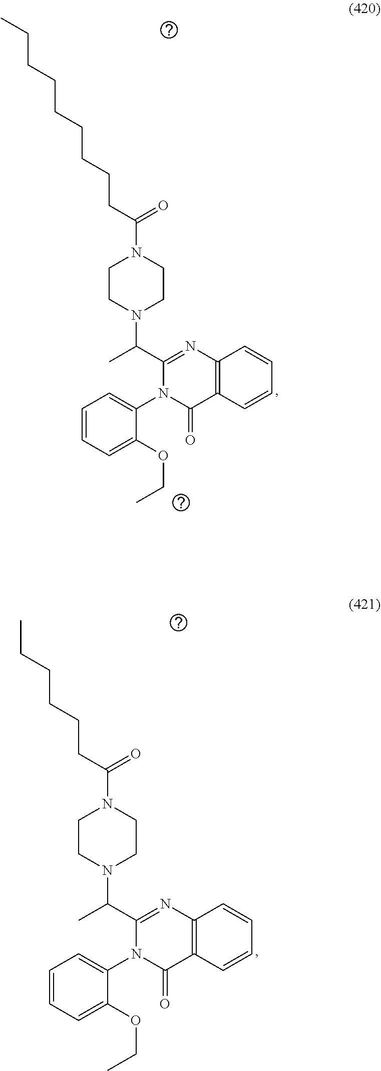 [SMILES: CCCCCCCCCC(=O)N1CCN(C(C)c2nc3ccccc3c(=O)n2-c2ccccc2OCC)CC1 ; CCCCCCC(=O)N1CCN(C(C)c2nc3ccccc3c(=O)n2-c2ccccc2OCC)CC1]

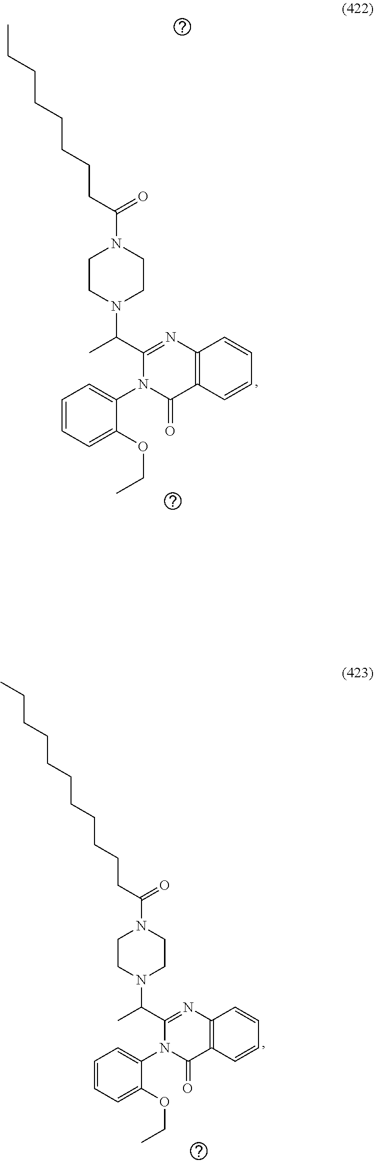 [SMILES: CCCCCCCCC(=O)N1CCN(C(C)c2nc3ccccc3c(=O)n2-c2ccccc2OCC)CC1 ; CCCCCCCCCCCC(=O)N1CCN(C(C)c2nc3ccccc3c(=O)n2-c2ccccc2OCC)CC1]

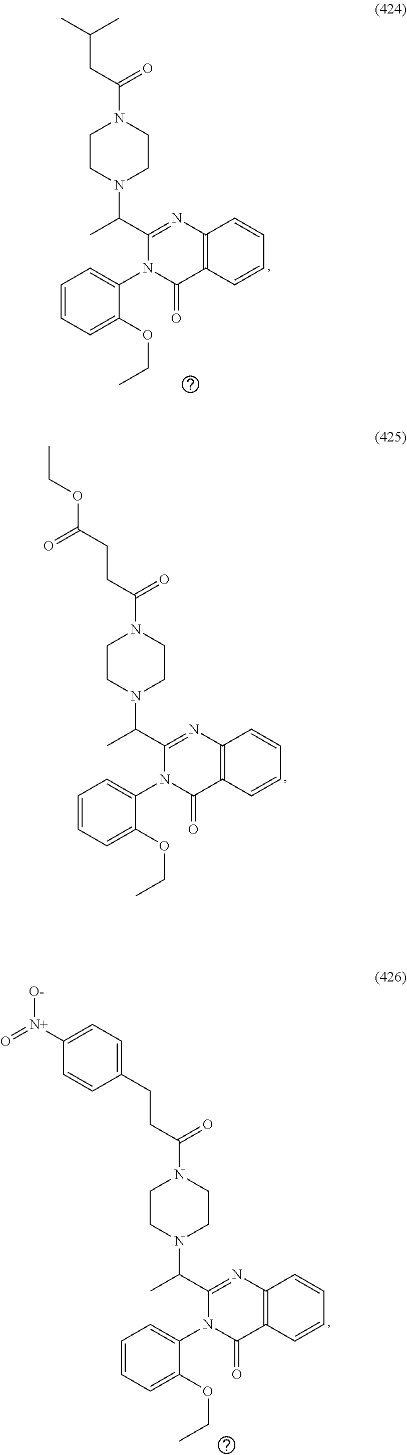 [SMILES: CCOc1ccccc1-n1c(C(C)N2CCN(C(=O)CC(C)C)CC2)nc2ccccc2c1=O ; CCOC(=O)CCC(=O)N1CCN(C(C)c2nc3ccccc3c(=O)n2-c2ccccc2OCC)CC1 ; CCOc1ccccc1-n1c(C(C)N2CCN(C(=O)CCc3ccc([N+](=O)[O-])cc3)CC2)nc2ccccc2c1=O]

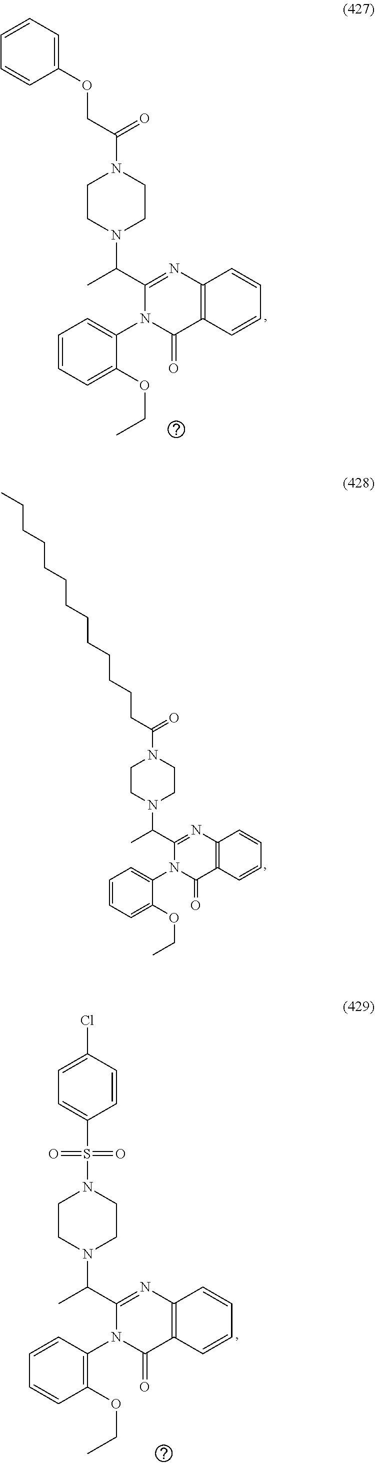 [SMILES: CCOc1ccccc1-n1c(C(C)N2CCN(C(=O)COc3ccccc3)CC2)nc2ccccc2c1=O ; CCCCCCCCCCCCCC(=O)N1CCN(C(C)c2nc3ccccc3c(=O)n2-c2ccccc2OCC)CC1 ; CCOc1ccccc1-n1c(C(C)N2CCN(S(=O)(=O)c3ccc(Cl)cc3)CC2)nc2ccccc2c1=O]

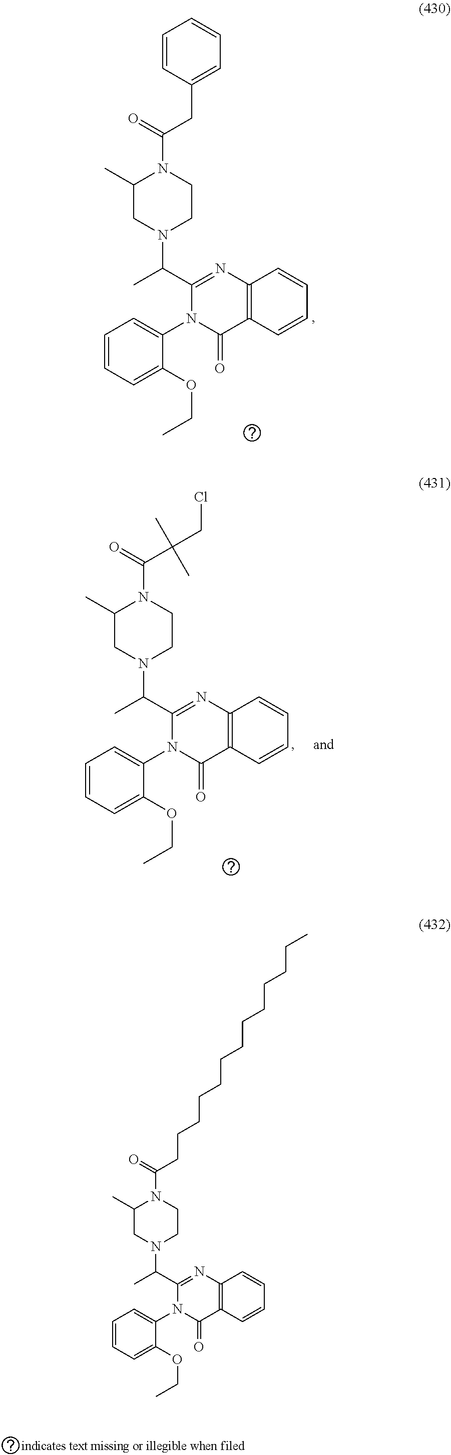 [SMILES: CCOc1ccccc1-n1c(C(C)N2CCN(C(=O)Cc3ccccc3)C(C)C2)nc2ccccc2c1=O ; CCOc1ccccc1-n1c(C(C)N2CCN(C(=O)C(C)(C)CCl)C(C)C2)nc2ccccc2c1=O ; CCCCCCCCCCCCCC(=O)N1CCN(C(C)c2nc3ccccc3c(=O)n2-c2ccccc2OCC)CC1C]

and pharmaceutically acceptable salts and esters thereof. In some embodiments, a compound selected from Compounds 314-432 is a VDAC modulator.

In some embodiments, the mitochondrial lipid shunt modulator is a compound disclosed in U.S. Patent Publication No. 2007/0161644A1 (e.g., compound VI, or a salt or derivative thereof), incorporated herein by reference in its entirety. In some embodiments, the mitochondrial lipid shunt modulator is a compound of Formula (XDX):

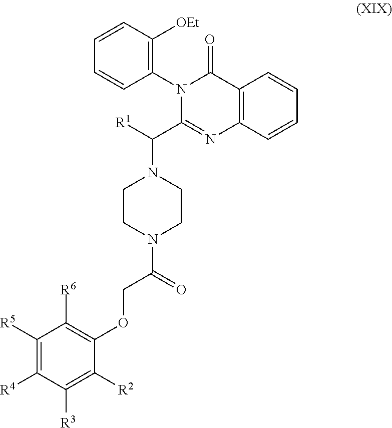

wherein R¹ is selected from H, C₁₋₈alkyl, C₁₋₈alkoxy, 3- to 8-membered carbocyclic or heterocyclic, aryl, heteroaryl, C₁₋₄ aralkyl, residues of glycolic acid, ethylene glycol/propylene glycol copolymers, carboxylate, ester, amide, carbohydrate, amino acid, alditol, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether; R², R³, R⁴, RV, and R⁶ are independently selected from H, halo, C₁₋₄alkyl, C₁₋₄alkylamino, acyl, and alkylsulfonyl; R⁷ is selected from H, C₁₋₈alkyl, optionally substituted carbocycle, aryl, heteroaryl, heterocycle, alkylaryl, alkylheteroaryl, and alkylheterocycle; R⁸ is selected from optionally substituted C₁₋₈alkyl, C₁₋₈alkenyl, C₁₋₈ alkynyl, aryl, carbocycle, heteroaryl, heterocycle, alkylaryl, alkylheteroaryl, alkylheterocycle, and heteroaromatic. In some embodiments, R¹ is not methyl when R⁴ is Cl, or an enantiomer, optical isomer, diastereomer, N-oxide, crystalline form, hydrate, or pharmaceutically acceptable salt thereof.

In some embodiments, the mitochondrial lipid shunt modulator of Formula (XIX) is a VDAC inhibitor. In some embodiments, the mitochondrial lipid shunt modulator of Formula (XIX) is selected from:

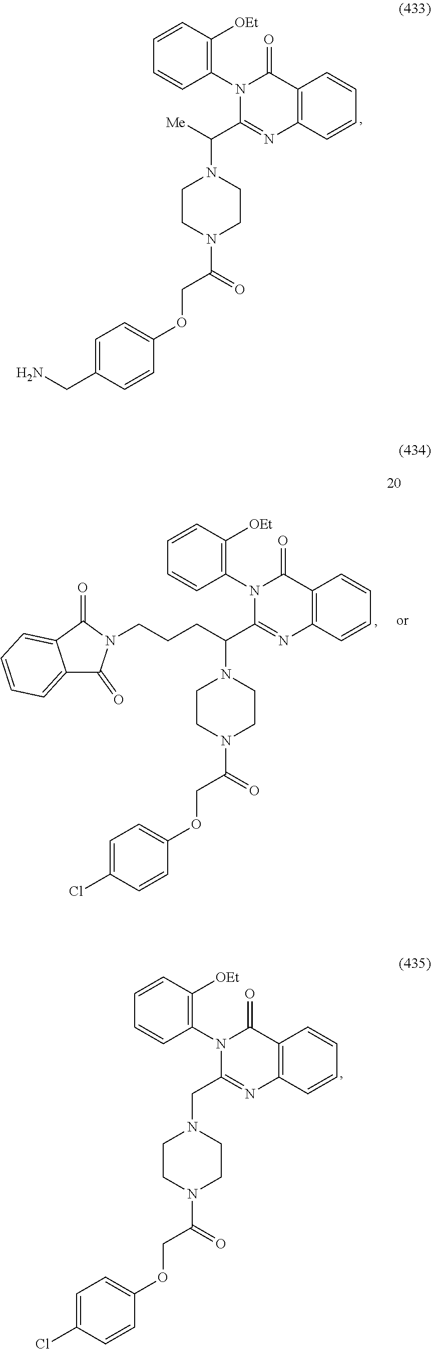 [SMILES: CCOc1ccccc1-n1c(C(C)N2CCN(C(=O)COc3ccc(CN)cc3)CC2)nc2ccccc2c1=O ; CCOc1ccccc1-n1c(C(CCCN2C(=O)c3ccccc3C2=O)N2CCN(C(=O)COc3ccc(Cl)cc3)CC2)nc2ccccc2c1=O ; CCOc1ccccc1-n1c(CN2CCN(C(=O)COc3ccc(Cl)cc3)CC2)nc2ccccc2c1=O]

and pharmaceutically acceptable salts and esters thereof.

In some embodiments, the mitochondrial lipid shunt modulator is a compound as disclosed in Canadian Patent No. 2,595,848 (e.g., compound V, or a salt or derivative thereof), incorporated herein by reference in its entirety. In some embodiments, the mitochondrial lipid shunt modulator is a compound of Formula (XX):

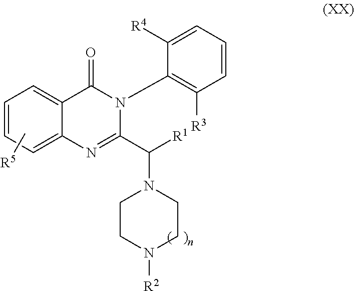

wherein R¹ is selected from H and C₁₋₈ alkyl; R² is selected from H and C₁₋₈ alkyl wherein C₁₋₈-alkyl is optionally substituted by one or more of hydroxyl, halo, cyano, alkoxy, nitro, carbonyl, thiocarbonyl, phosphoryl, phosphate, phosphonate, phosphinate, amino, amido, amidine, imine, azido, sulfhydryl, alkylthio, sulfate, sulfonate, sulfamoyl, sulfamido, sulfonyl and heterocyclyl; R³ is selected from alkoxy and unsubstituted C₁₋₈alkyl; R⁴ is selected from H, alkoxy and unsubstituted C₁₋₈alkyl; R₅ is selected from H, halogen; and n is 1 or 2.

In some embodiments, the compound of Formula (XX) is a VDAC inhibitor. In some embodiments, the compound of Formula (XX) is selected from:

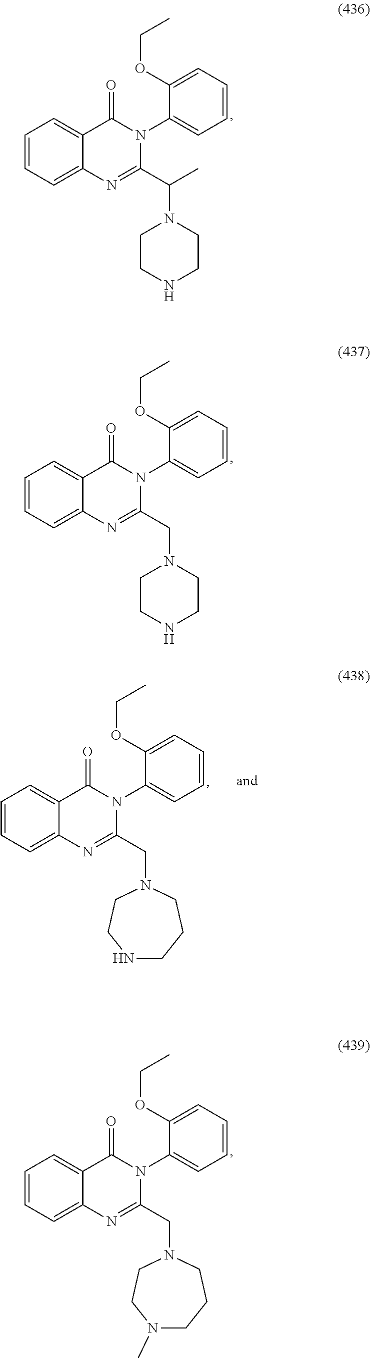 [SMILES: CCOc1ccccc1-n1c(C(C)N2CCNCC2)nc2ccccc2c1=O ; CCOc1ccccc1-n1c(CN2CCNCC2)nc2ccccc2c1=O ; CCOc1ccccc1-n1c(CN2CCCNCC2)nc2ccccc2c1=O ; CCOc1ccccc1-n1c(CN2CCCN(C)CC2)nc2ccccc2c1=O]

or pharmaceutically acceptable salts and esters thereof.

In some embodiments, the mitochondrial lipid shunt modulator is a compound disclosed U.S. Patent Publication No. 2017/0362173 (e.g., Efsevin or a salt or derivative thereof), incorporated herein by reference in its entirety. In some embodiments, the mitochondrial lipid shunt modulator is:

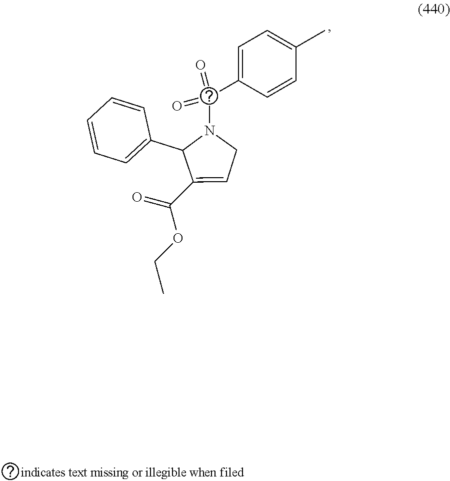

and pharmaceutically acceptable salts and esters thereof.

In some embodiments, the mitochondrial lipid shunt modulator of U.S. Patent Publication No. 2017/0362173 is a VDAC inhibitor. In some embodiments, the mitochondrial lipid shunt modulator is selected from:

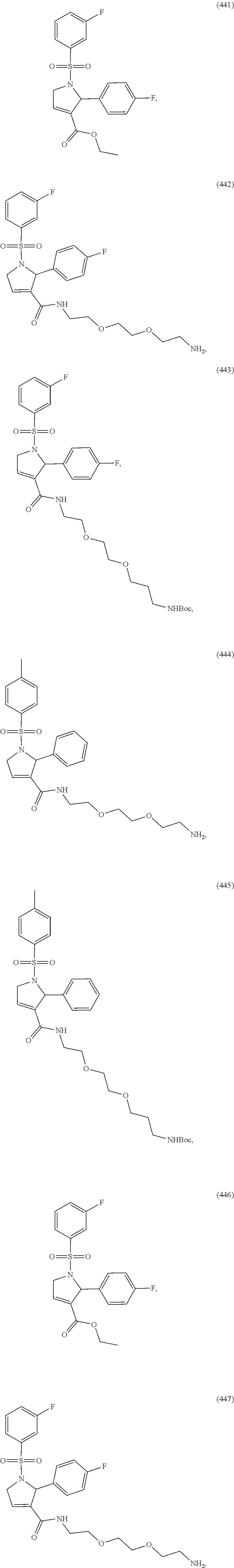

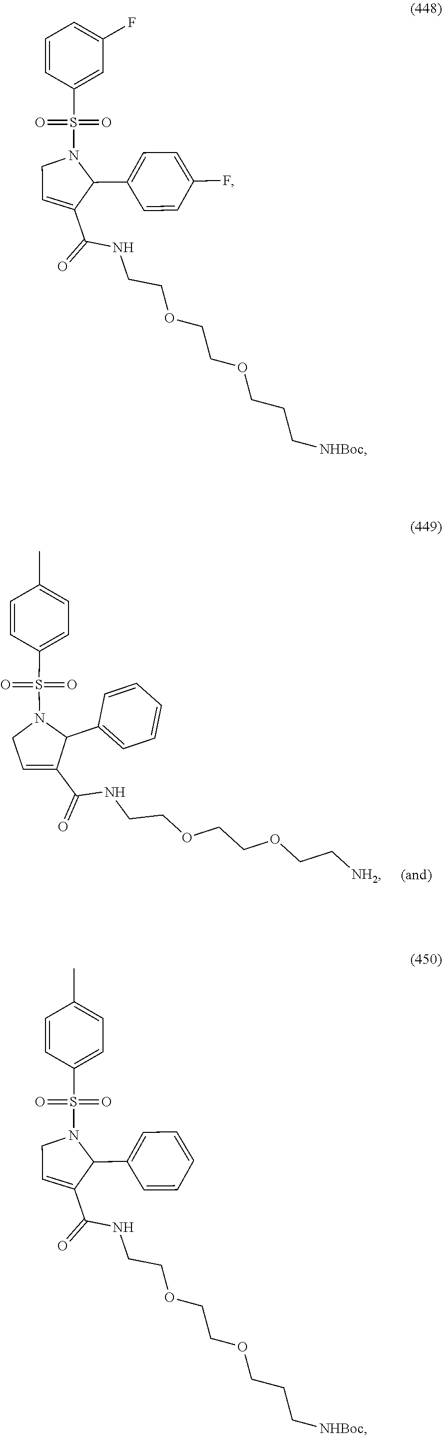 [SMILES: CC(C)(C)OC(=O)NCCCOCCOCCNC(=O)C1=CCN(S(=O)(=O)c2cccc(F)c2)C1c1ccc(F)cc1 ; Cc1ccc(S(=O)(=O)N2CC=C(C(=O)NCCOCCOCCN)C2c2ccccc2)cc1 ; Cc1ccc(S(=O)(=O)N2CC=C(C(=O)NCCOCCOCCCNC(=O)OC(C)(C)C)C2c2ccccc2)cc1]

and pharmaceutically acceptable salts and esters thereof.

In some embodiments, the mitochondrial lipid shunt modulator is a compound disclosed in Gianessi et al (J. Med Chem (2001) 44(15):2383-2386 or J. Med Chem (2003) 46:303-309 e.g., an aminocarnitine derivative or a salt thereof), each of which is incorporated herein by reference in its entirety. In some embodiments, the mitochondrial lipid shunt modulator is selected from:

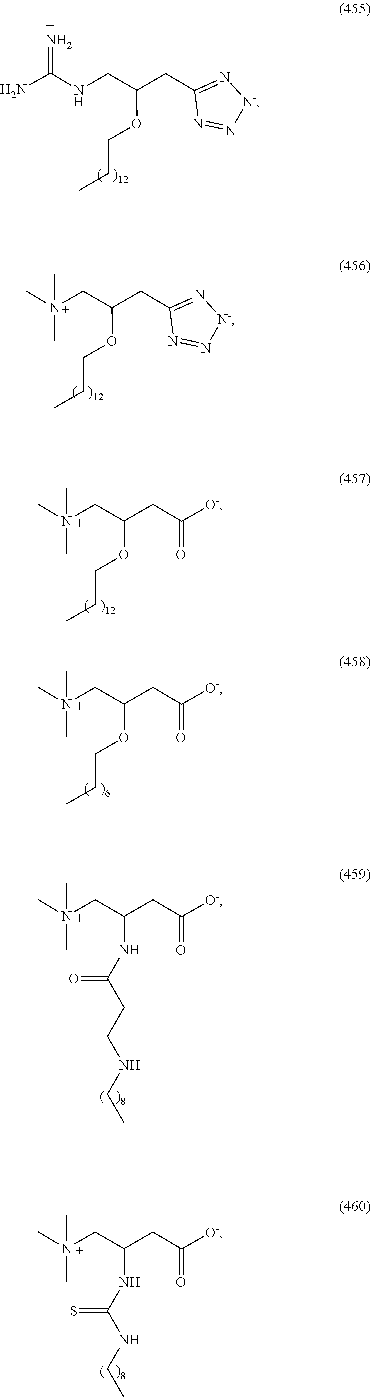

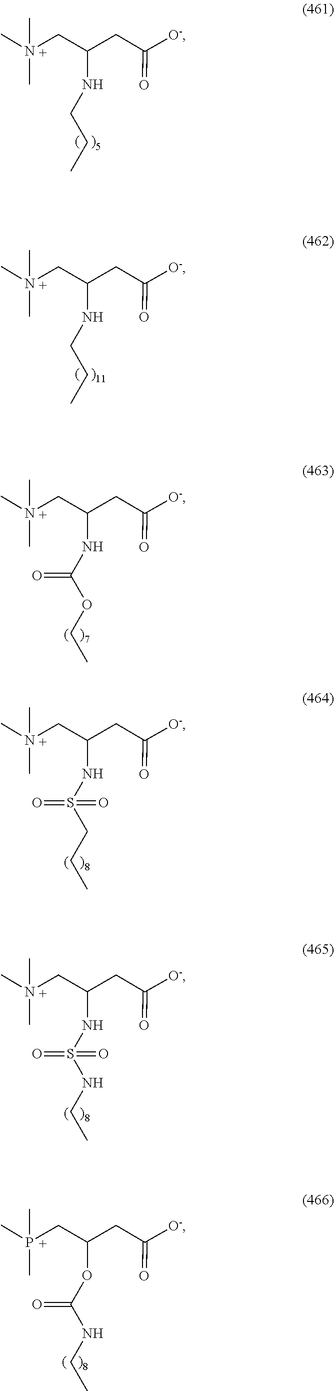

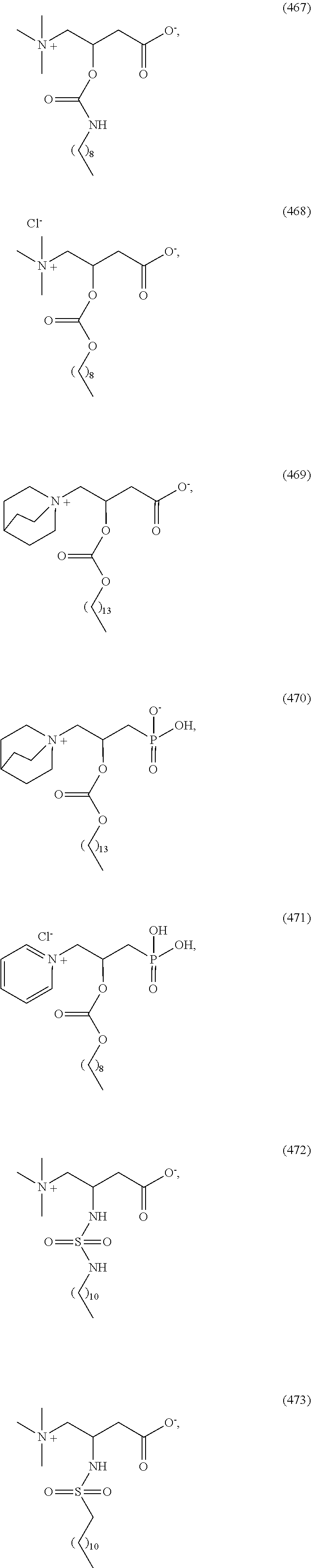

and pharmaceutically acceptable salts and esters thereof. In some embodiments, the mitochondrial lipid shunt modulator selected from Compound 455-473 is chiral (e.g., an S or R isomer). In some embodiments, the mitochondrial lipid shunt modulator selected from Compound 455-473 is a CPT1 inhibitor.

In some embodiments, the mitochondrial lipid shunt modulator is a compound disclosed in Ben-Hail et al (J. Biol Chem (2016) 291(48):24986-25003), incorporated herein by reference in its entirety. In some embodiments, the mitochondrial lipid shunt modulator is a compound disclosed in U.S. Pat. No. 10,434,099, incorporated herein by reference in its entirety. In some embodiments, the mitochondrial lipid shunt modulator is selected from:

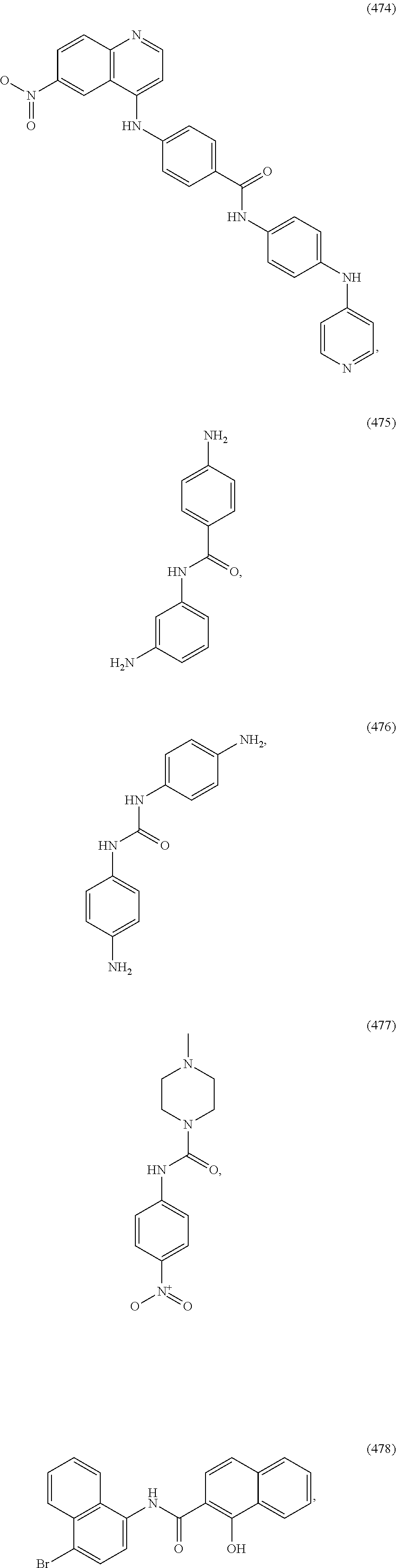

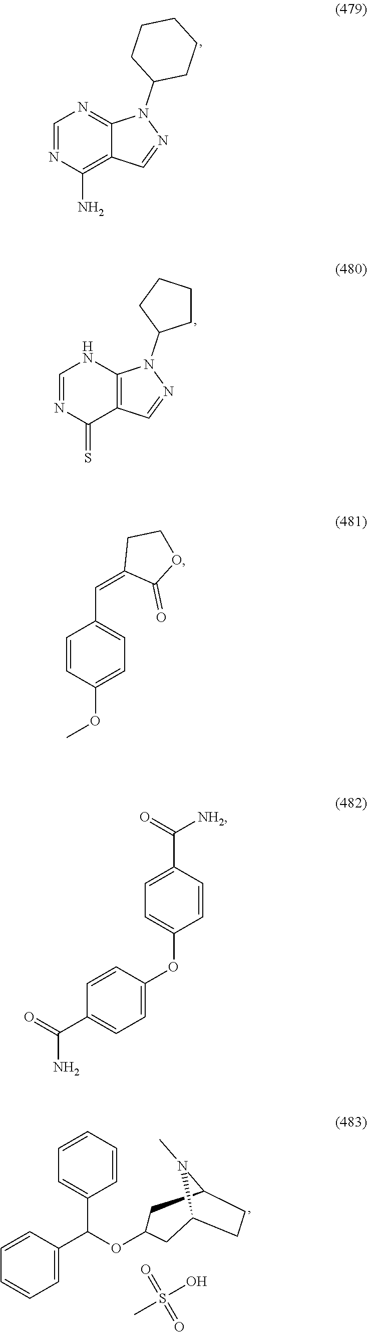

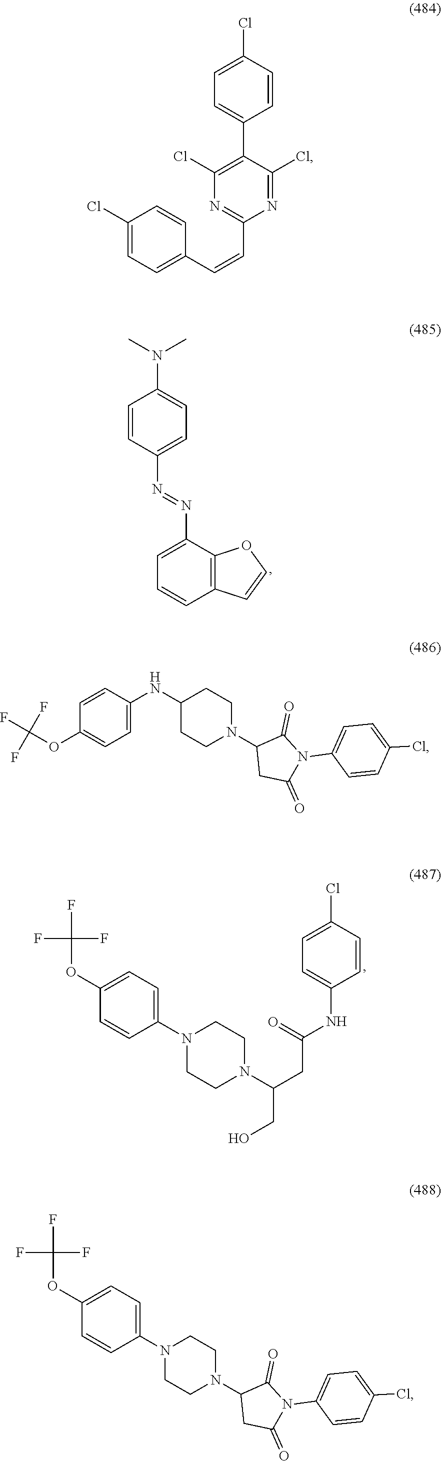 [SMILES: Clc1ccc(/C=C\c2nc(Cl)c(-c3ccc(Cl)cc3)c(Cl)n2)cc1 ; CN(C)c1ccc(/N=N/c2cccc3ccoc23)cc1 ; O=C1CC(N2CCC(Nc3ccc(OC(F)(F)F)cc3)CC2)C(=O)N1c1ccc(Cl)cc1 ; O=C(CC(CO)N1CCN(c2ccc(OC(F)(F)F)cc2)CC1)Nc1ccc(Cl)cc1 ; O=C1CC(N2CCN(c3ccc(OC(F)(F)F)cc3)CC2)C(=O)N1c1ccc(Cl)cc1]

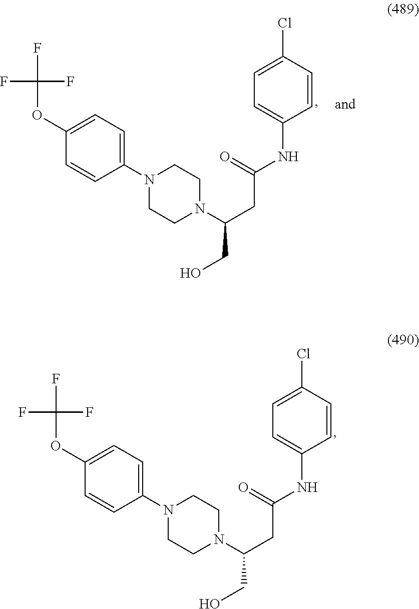 [SMILES: O=C(C[C@@H](CO)N1CCN(c2ccc(OC(F)(F)F)cc2)CC1)Nc1ccc(Cl)cc1 ; O=C(C[C@H](CO)N1CCN(c2ccc(OC(F)(F)F)cc2)CC1)Nc1ccc(Cl)cc1]

pharmaceutically acceptable salts and esters thereof. In some embodiments, the mitochondrial lipid shunt modulator selected from Compound 474-490 is a VDAC inhibitor (e.g., VDAC1 inhibitor).

In some embodiments, the mitochondrial lipid shunt modulator is a non-steroidal anti-inflammatory drug (NSAID). In some embodiments, the mitochondrial lipid shunt modulator is a compound disclosed in Y. Aono et al (Biochem. Biophys. Res. Commun. (2018) 505(4):1-8); E. Gurpinar et al. (Frontiers in Oncology (2013) 3: 181); or U.S. Pat. No. 3,654,349, or a salt or derivative thereof, each of which is incorporated herein by reference in its entirety. In some embodiments, the mitochondrial lipid shunt modulator is a compound of Formula (XXI):

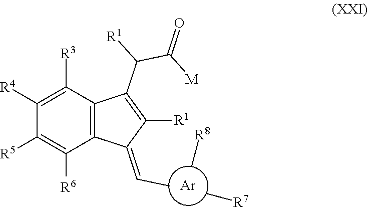

wherein AR is aryl or heteroaryl; R¹ is hydrogen, lower wherein: Ar may be aryl or heteroaryl; R₁ may be hydrogen, loweralkyl or halogenated lower alkyl; R² may be hydrogen or alkyl; R³, R⁴, R⁵ and R⁶ each may be hydrogen, alkyl, acyloxy, alkoxy, nitro, amino, acylamino, alkylamino, dialkyl amino, dialkylaminoalkyl, sulfanyl, alkythio, mercapto, hydroxy, hydroxyalkyl, alkylsulfonyl, halogen, cyano, carboxyl, carbalkoxy, carbamido, halogen, alkyl, cycloalkyl or cycloalkoxy; R⁷ may be alkylthio, alkylsulfinyl or alkylsulfonyl; R⁸ may be hydrogen, halogen, hydroxy, alkoxy, or halo alkyl; and M may be hydroxy, loweralkoxy, substituted loweralkoxy, amino, alkylamino, dialkylamino, N-morpholino, hydroxyalkylamino, polyhydroxyalkylamino, dialkylaminoalkylamino, or aminoalkylamino.

In some embodiments, the mitochondrial lipid shunt modulator is sulindac or a metabolite or derivative thereof, or a pharmaceutically acceptable salt thereof. In some embodiments, the mitochondrial lipid shunt modulator comprises sulindac sulfide, sulindac sulfoxide, or sulindac sulfone (i.e., exisulind). In some embodiments, the mitochondrial lipid shunt modulator is selected from the group comprising:

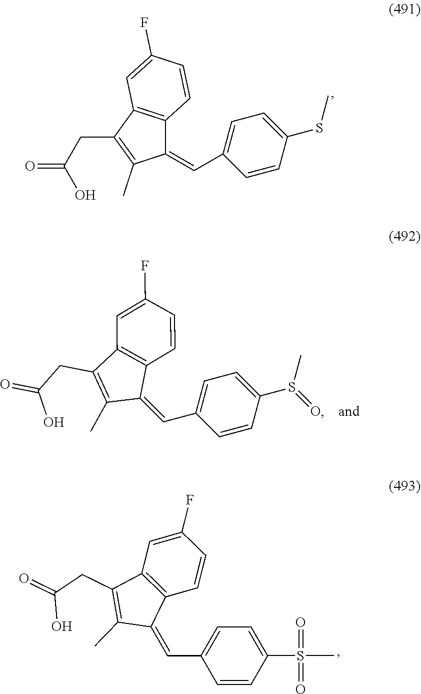 [SMILES: CSc1ccc(/C=C2/C(C)=C(CC(=O)O)c3cc(F)ccc32)cc1 ; CC1=C(CC(=O)O)c2cc(F)ccc2/C1=C\c1ccc(S(C)=O)cc1 ; "and" ; CC1=C(CC(=O)O)c2cc(F)ccc2/C1=C\c1ccc(S(C)(=O)=O)cc1]

or a pharmaceutically acceptable salt thereof.

In some embodiments, the mitochondrial lipid shunt modulator is a SRC Kinase inhibitor. In some embodiments, the mitochondrial lipid shunt modulator is a compound disclosed in Amen et al. (Frontiers in Cell and Developmental Biology (2021) 8: 606111) In some embodiments, the mitochondrial lipid shunt modulator is 1-NA-PP1 or a pharmaceutically acceptable salt thereof. n some embodiments, the compounds is

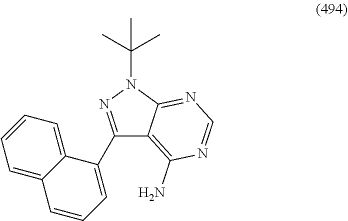 [SMILES: CC(C)(C)n1nc(-c2cccc3ccccc23)c2c(N)ncnc21]

or a pharmaceutically acceptable salt thereof.

In some embodiments, the mitochondrial lipid shunt modulator is an anti-fungal compound. In some embodiments, the mitochondrial lipid shunt modulator is a compound disclosed in Head et al. (Proceedings of the National Academy of Sciences (2015) 112(52): E7276-E7285); or U.S. Pat. No. 4,267,179, or a salt or derivative thereof, each of which is incorporated herein by reference in its entirety. In some embodiments, the mitochondrial lipid shunt modulator is a compound of Formula (XXII):

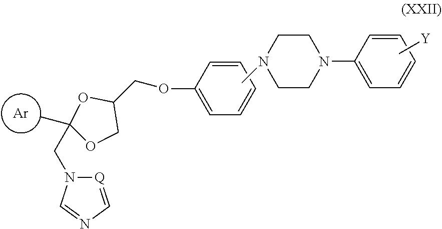

or a pharmaceutically acceptable salt thereof, wherein: Q is a member selected from the group consisting of CH and N; Ar is a member selected from the group consisting of phenyl, thienyl, halothienyl and substituted phenyl, said substituted phenyl having from 1 to 3 substituents each independently selected from the group consisting of halo, lower alkyl, lower alkyloxy and trifluoromethyl; and the radical Y is a 2,3-dihydro-4H-1,2,4-triazol-4-yl radical of the formula

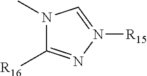

wherein R¹⁵ is selected from the group consisting of lower alkyl and aryl lower alkyl and R¹⁶ is selected from the group consisting of hydrogen, lower alkyl, and aryl; wherein said aryl as used in the foregoing definition is selected from the group consisting of phenyl and substituted phenyl, said substituted phenyl having from 1 to 3 substituents each independently selected from the group consisting of halo, lower alkyl, lower alkyloxy and trifluoromethyl.

In some embodiments, the mitochondrial lipid shunt modulator is itraconazole or a stereoisomer, metabolite or derivative thereof, or a pharmaceutically acceptable salt thereof. In some embodiments, the mitochondrial lipid shunt modulator comprises

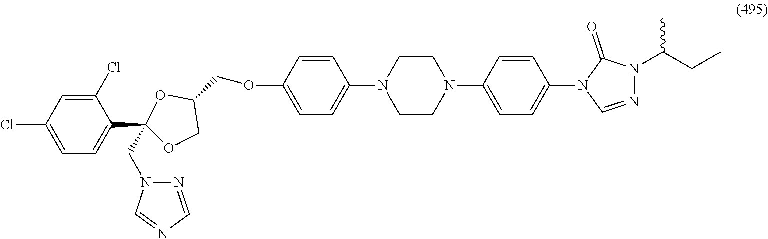 [SMILES: CCC(C)n1ncn(-c2ccc(N3CCN(c4ccc(OC[C@@H]5CO[C@@](Cn6cncn6)(c6ccc(Cl)cc6Cl)O5)cc4)CC3)cc2)c1=O]

or a pharmaceutically acceptable salt thereof. In some embodiments, the mitochondrial lipid shunt modulator comprises:

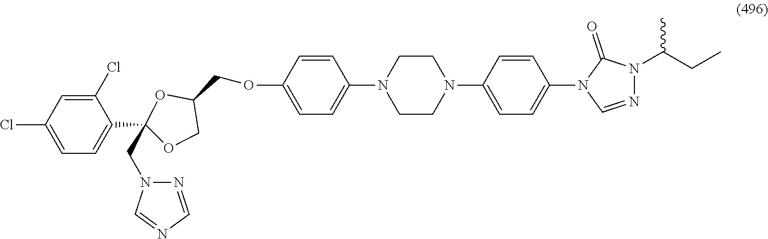 [SMILES: CCC(C)n1ncn(-c2ccc(N3CCN(c4ccc(OC[C@H]5CO[C@](Cn6cncn6)(c6ccc(Cl)cc6Cl)O5)cc4)CC3)cc2)c1=O]

or a pharmaceutically acceptable salt thereof. In an embodiment, the itraconazole is administered as a mixture of isomers.

In some embodiments, the mitochondrial lipid shunt modulator is a compound disclosed in Hayashi et al. (Bioorg. Med. Chem. Lett. (2021) 33: 127722), or a salt or derivative thereof, which is incorporated herein by reference in its entirety. In some embodiments, the mitochondrial lipid shunt modulator is a compound selected from the group comprising:

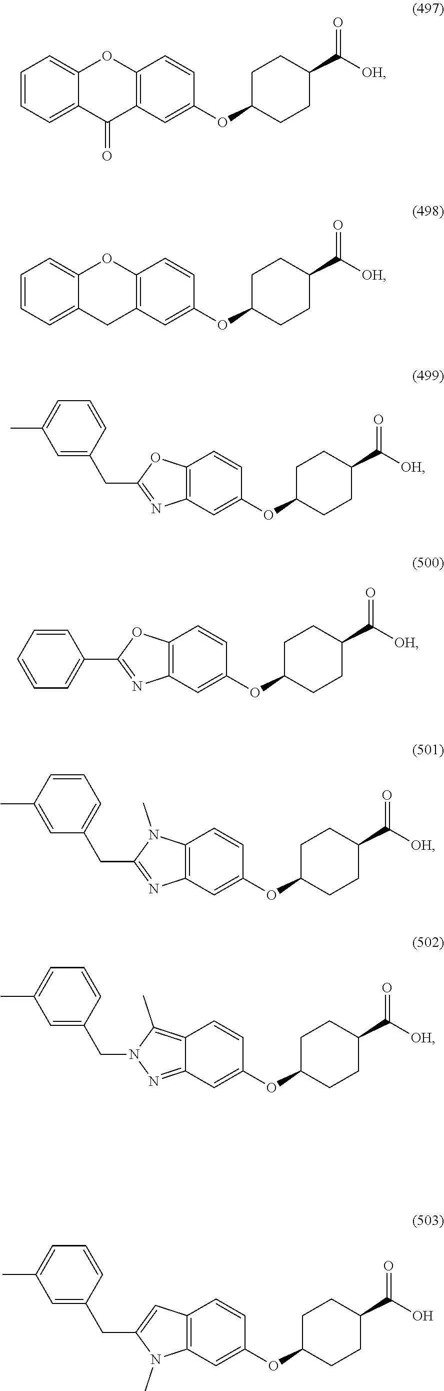

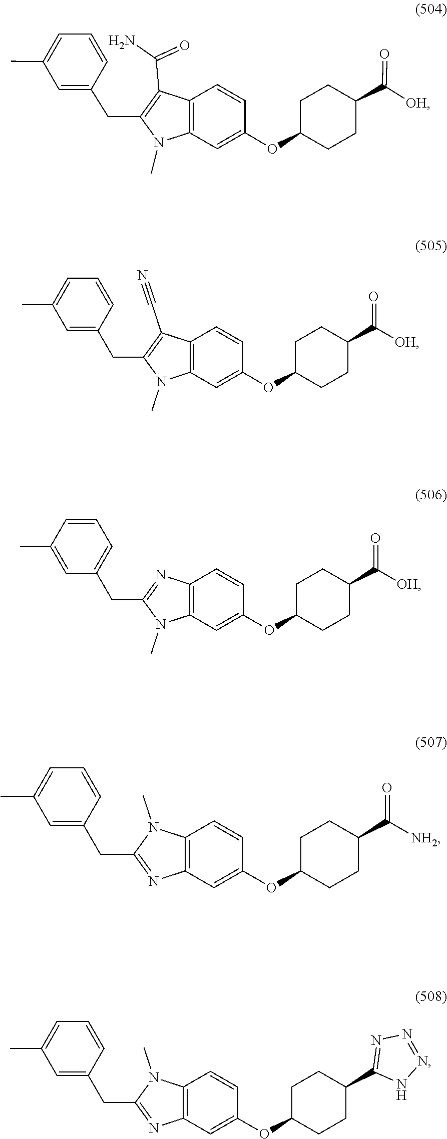 [SMILES: Cc1cccc(Cc2c(C(N)=O)c3ccc(O[C@H]4CC[C@@H](C(=O)O)CC4)cc3n2C)c1 ; Cc1cccc(Cc2c(C#N)c3ccc(O[C@H]4CC[C@@H](C(=O)O)CC4)cc3n2C)c1 ; Cc1cccc(Cc2nc3ccc(O[C@H]4CC[C@@H](C(=O)O)CC4)cc3n2C)c1 ; Cc1cccc(Cc2nc3cc(O[C@H]4CC[C@@H](C(N)=O)CC4)ccc3n2C)c1 ; Cc1cccc(Cc2nc3cc(O[C@H]4CC[C@@H](c5nnn[nH]5)CC4)ccc3n2C)c1]

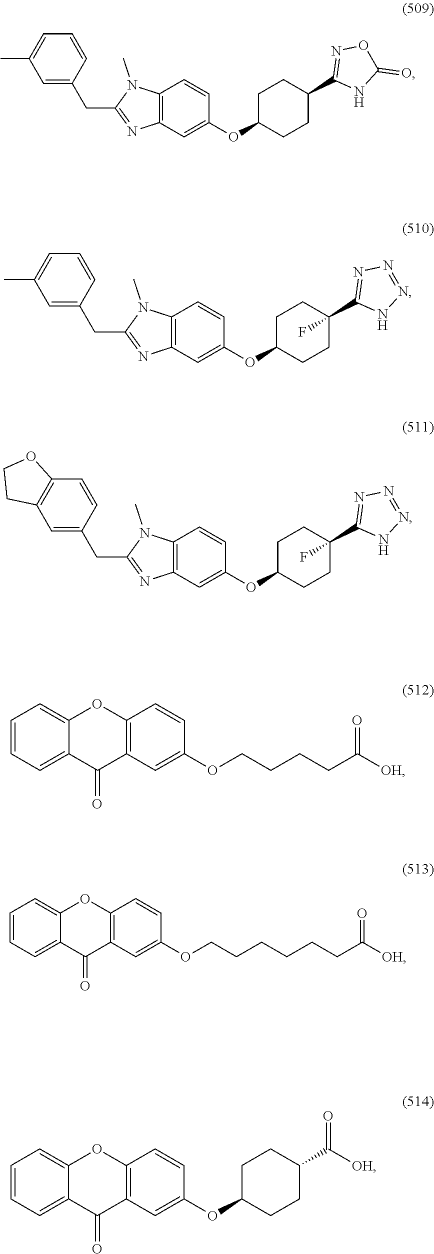

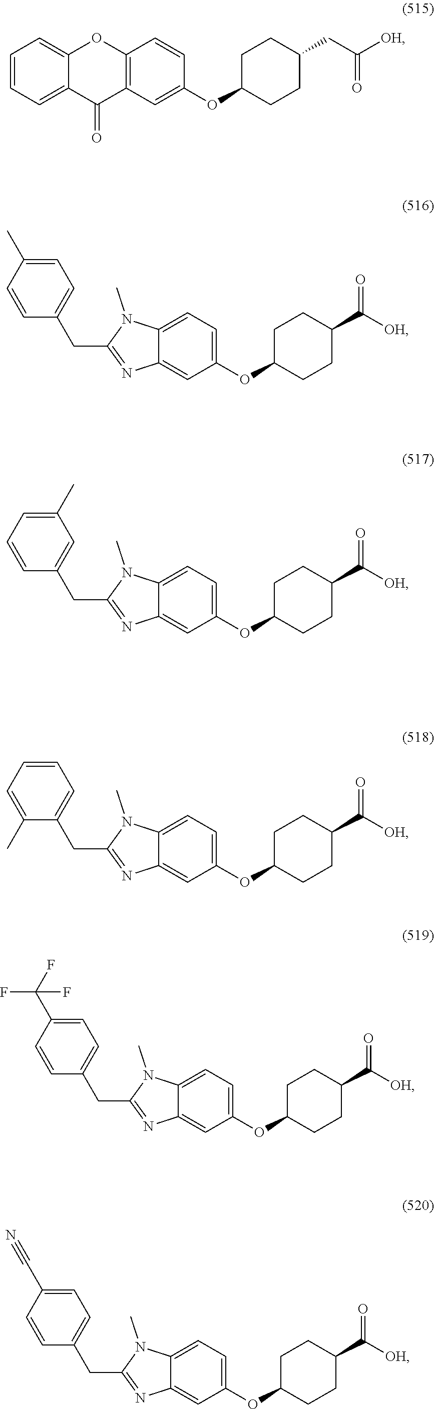 [SMILES: O=C(O)C[C@H]1CC[C@H](Oc2ccc3oc4ccccc4c(=O)c3c2)CC1 ; Cc1ccc(Cc2nc3cc(O[C@H]4CC[C@@H](C(=O)O)CC4)ccc3n2C)cc1 ; Cc1cccc(Cc2nc3cc(O[C@H]4CC[C@@H](C(=O)O)CC4)ccc3n2C)c1 ; Cc1ccccc1Cc1nc2cc(O[C@H]3CC[C@@H](C(=O)O)CC3)ccc2n1C ; Cn1c(Cc2ccc(C(F)(F)F)cc2)nc2cc(O[C@H]3CC[C@@H](C(=O)O)CC3)ccc21 ; Cn1c(Cc2ccc(C#N)cc2)nc2cc(O[C@H]3CC[C@@H](C(=O)O)CC3)ccc21]

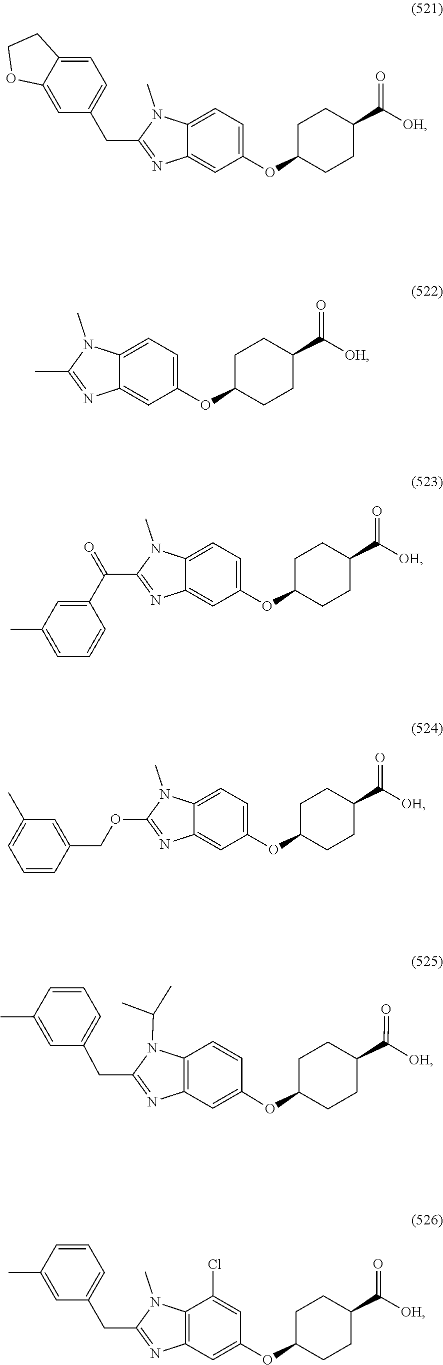 [SMILES: Cn1c(Cc2ccc3c(c2)OCC3)nc2cc(O[C@H]3CC[C@@H](C(=O)O)CC3)ccc21 ; Cc1nc2cc(O[C@H]3CC[C@@H](C(=O)O)CC3)ccc2n1C ; Cc1cccc(C(=O)c2nc3cc(O[C@H]4CC[C@@H](C(=O)O)CC4)ccc3n2C)c1 ; Cc1cccc(COc2nc3cc(O[C@H]4CC[C@@H](C(=O)O)CC4)ccc3n2C)c1 ; Cc1cccc(Cc2nc3cc(O[C@H]4CC[C@@H](C(=O)O)CC4)ccc3n2C(C)C)c1 ; Cc1cccc(Cc2nc3cc(O[C@H]4CC[C@@H](C(=O)O)CC4)cc(Cl)c3n2C)c1]

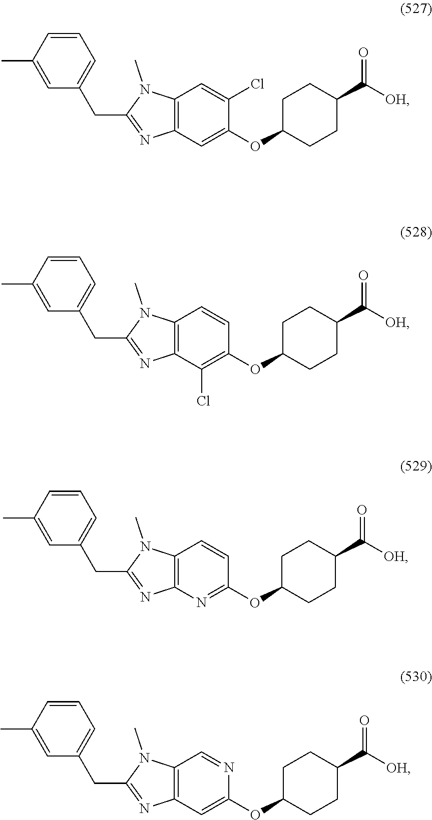 [SMILES: Cc1cccc(Cc2nc3cc(O[C@H]4CC[C@@H](C(=O)O)CC4)c(Cl)cc3n2C)c1 ; Cc1cccc(Cc2nc3c(Cl)c(O[C@H]4CC[C@@H](C(=O)O)CC4)ccc3n2C)c1 ; Cc1cccc(Cc2nc3nc(O[C@H]4CC[C@@H](C(=O)O)CC4)ccc3n2C)c1 ; Cc1cccc(Cc2nc3cc(O[C@H]4CC[C@@H](C(=O)O)CC4)ncc3n2C)c1]

or a pharmaceutically acceptable salt or derivative thereof.

In some embodiments, the mitochondrial lipid shunt modulator is a ErbB2 Kinase inhibitor. In some embodiments, the mitochondrial lipid shunt modulator is tyrphostin AG-825 or a pharmaceutically acceptable salt thereof. In some embodiments, the compound is

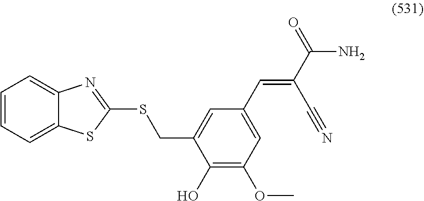 [SMILES: COc1cc(/C=C(\C#N)C(N)=O)cc(CSc2nc3ccccc3s2)c1O]

or a pharmaceutically acceptable salt thereof.

In some embodiments, the mitochondrial lipid shunt modulator is a FASN inhibitor. In some embodiments, the mitochondrial lipid shunt modulator is FASNALL or a pharmaceutically acceptable salt thereof. In some embodiments, the compound is

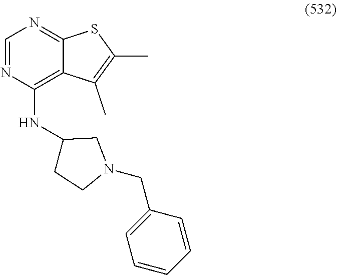 [SMILES: Cc1sc2ncnc(NC3CCN(Cc4ccccc4)C3)c2c1C]

or a pharmaceutically acceptable salt thereof.

In some embodiments, the mitochondrial lipid shunt modulator is an mTOR inhibitor compound. In some embodiments, the mitochondrial lipid shunt modulator is a compound disclosed in U.S. Pat. No. 8,394,818, or a salt or derivative thereof, which is incorporated herein by reference in its entirety. In some embodiments, the mitochondrial lipid shunt modulator is a compound of Formula (XXIII):

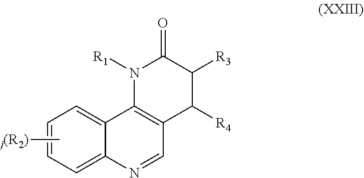

or a pharmaceutically acceptable salt thereof, wherein:
R¹ is an optionally substituted group selected from the group consisting of 6-10-membered aryl; C₇₋₁₅ arylalkyl; C₆₋₁₅ heteroarylalkyl; C₁₋₁₂ heteroaliphatic; C₁₋₁₂ aliphatic; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur, each occurrence of R is independently halogen, —NR₂—OR, —SR, or an optionally substituted group selected from the group consisting of C₁₋₁₂ acyl; 6-10-membered aryl; C₇₋₁₅ arylalkyl; C₆₋₁₅ heteroarylalkyl; C₁₋₁₂ heteroaliphatic; C₁₋₁₂ aliphatic; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; j is an integer from 1 to 4, inclusive; R³ and R⁴ are independently hydrogen, hydroxyl, alkoxy, halogen, or optionally substituted C₁₋₆ aliphatic, with the proviso that R³ and R⁴ are not taken together to form a ring; and each R is independently hydrogen, an optionally substituted group selected from the group consisting of C₁₋₁₂ acyl; 6-10-membered aryl; C₇₋₁₅ arylalkyl; C₆₋₁₅ heteroarylalkyl; C₁₋₁₂ aliphatic; 5-10-membered heteroaryl having 1-4 heteroatoms independently selected from the group consisting of nitrogen, oxygen, and sulfur; 4-7-membered heterocyclic having 1-2 heteroatoms independently selected from the group consisting of nitrogen, oxygen, and sulfur; and C₁₋₁₂ heteroaliphatic having 1-2 heteroatoms independently selected from the group consisting of nitrogen, oxygen, and sulfur; or: two R on the same nitrogen atom are taken with the nitrogen to form a 4-7-membered heterocyclic ring having 1-2 heteroatoms independently selected from the group consisting of nitrogen, oxygen, and sulfur.

In some embodiments, the mitochondrial lipid shunt modulator is an mTOR inhibitor. In some embodiments, the mitochondrial lipid shunt modulator is Torin 1 or a pharmaceutically acceptable salt thereof. In some embodiments, the compound is

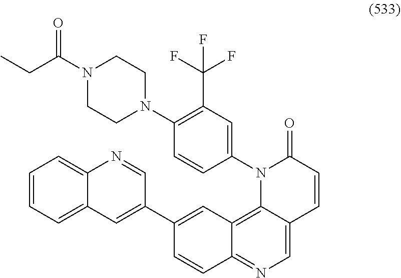 [SMILES: CCC(=O)N1CCN(c2ccc(-n3c(=O)ccc4cnc5ccc(-c6cnc7ccccc7c6)cc5c43)cc2C(F)(F)F)CC1]

or a pharmaceutically acceptable salt thereof.

In some embodiments, the mitochondrial lipid shunt modulator is a tyrosine kinase inhibitor. In some embodiments, the mitochondrial lipid shunt modulator is BIBF 1120 (i.e., nintedanib), or a stereoisomer, metabolite or derivative thereof, or a pharmaceutically acceptable salt thereof. In some embodiments, the mitochondrial lipid shunt modulator comprises

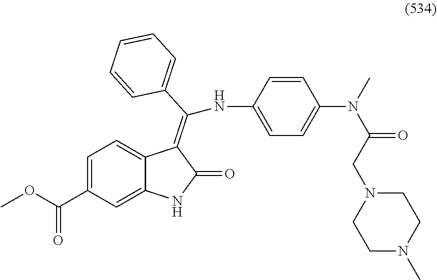

or a pharmaceutically acceptable salt thereof.

In some embodiments, the mitochondrial lipid shunt modulator is a dual PI3K/mTOR inhibitor compound. In some embodiments, the mitochondrial lipid shunt modulator is GSK1059615 or a pharmaceutically acceptable salt thereof. In some embodiments, the compound is

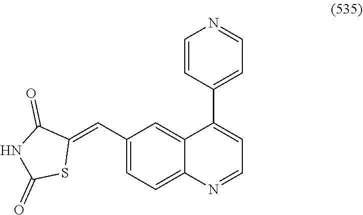 [SMILES: O=C1NC(=O)/C(=C/c2ccc3nccc(-c4ccncc4)c3c2)S1]

or a pharmaceutically acceptable salt thereof. In some embodiments, the mitochondrial lipid shunt modulator is 17p-hydroxywortmannin or a pharmaceutically acceptable salt thereof. In some embodiments, the mitochondrial lipid shunt modulator is

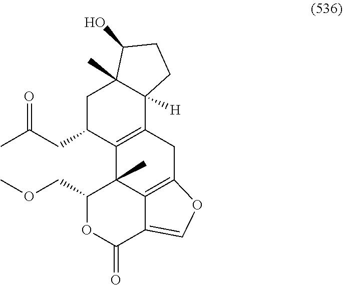

or a pharmaceutically acceptable salt thereof.

In some embodiments, the mitochondrial lipid shunt modulator is a Janus kinase inhibitor compound. In some embodiments, the mitochondrial lipid shunt modulator is a compound disclosed in U.S. Pat. No. 7,598,257, or a salt or derivative thereof, which is incorporated herein by reference in its entirety. In some embodiments, the mitochondrial lipid shunt modulator is a compound of Formula (XXIV):

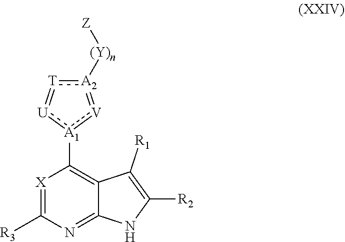

or a pharmaceutically acceptable salt thereof, wherein:
A¹ and A² are independently selected from C and N; T, U, and V are independently selected from O, S, N, CR⁵, and NR⁶; wherein the 5-membered ring formed by A¹, A², U, T, and V is aromatic; X is N or CR⁴; Y is C₁₋₈ alkylene, C₂₋₈ alkenylene, C₂₋₈ alkynylene, (CR¹¹R¹²)ₚ—(C₃₋₁₀ cycloalkylene)-(CR¹¹R¹²)_{q}, (CR¹¹R¹²)ₚ-(arylene)-(CR¹¹R¹²)_{q}, (CR¹¹R¹²)ₚ—(C₁₋₁₀ heterocycloalkylene)-(CR¹¹R¹²)_{q}, (CR¹¹R¹²)ₚ-(heteroarylene)-(CR¹¹R¹²)_{q}, (CR¹¹R¹²)ₚO(CR¹¹R¹²)_{q}, (CR¹¹R¹²)ₚS(CR¹¹R¹²)_{q}, (CR¹¹R¹²)ₚC(O)(CR¹¹R¹²)_{q}, (CR¹¹R¹²)ₚC(O)NR^{c}(CR¹¹R¹²)_{q}, (CR¹¹R¹²)ₚC(O)O(CR¹¹R¹²)_{q}, (CR¹¹R¹²)ₚOC(O)(CR¹¹R¹²)_{q}, (CR¹¹R¹²)ₚOC(O)NR^{c}(CR¹¹R¹²)_{q}, (CR¹¹R¹²)ₚNR^{c}(CR¹¹R¹²)_{q}, (CR¹¹R¹²)ₚNR^{c}(O)NR^{d}(CR¹¹R¹²)_{q}, (CR¹¹R¹²)ₚS(O)(CR¹¹R¹²)_{q}, (CR¹¹R¹²)ₚS(O)NR^{c}(CR¹¹R¹²)_{q}, (CR¹¹R¹²)ₚS(O)₂(CR¹¹R¹²)_{q}, or (CR¹¹R¹²)ₚS(O)₂NR^{c}(CR¹¹R¹²)_{q}, wherein said C₁₋₈ alkylene, C₂₋₈ alkenylene, C₂₋₈ alkynylene, cycloalkylene, arylene, heterocycloalkylene, or heteroarylene, is optionally substituted with 1, 2, or 3 substituents independently selected from -D¹-D²-D³-D⁴; Z is H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, ═C—Rⁱ, ═N—Rⁱ, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d} NR^{c}C(O)R^{b}, NR^{c}C(O)NR^{c}R^{d}, NR^{c}C(O)OR^{a}, C(═NR^{c})NR^{c}R^{d}, NR^{c}(═NRⁱ)NR^{c}R^{d}, S(O)R^{b}, S(O)NR^{c}R^{d}, S(O)₂R^{b}, NRS(O)₂R^{b}, C(═NOH)R^{b}, C(═NO(C₁₋₆ alkyl)R^{b}, and S(O)₂NR^{c}R^{d}, wherein said C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)R^{b}, C(═NOH)R^{b}, C(═NO(C₁₋₆ alkyl))R^{b}, and S(O)₂NR^{c}R^{d}; wherein when Z is H, n is b 1 or the —(Y)ₙ—Z moiety is taken together with 1) A² to which the moiety is attached, ii) R_{or R}⁶ of either T or V, and iii) the C or N atom to which the R⁵ or R⁶ of either T or V is attached to form a 4- to 20-membered aryl, cycloalkyl, heteroaryl, or heterocycloalkyl ring fused to the 5-membered ring formed by A¹, A², U, T, and V, wherein said 4- to 20-membered aryl, cycloalkyl, heteroaryl, or heterocycloalkyl ring is optionally substituted by 1, 2, 3, 4, or 5 substituents independently selected from —(W)ₘ-Q, W is C₁₋₈ alkylenyl, C₂₋₈ alkenylenyl, C₂₋₈ alkynylenyl, O, S, C(O), C(O)NR^{c′}, C(O)O, OC(O), OC(O)NR^{c′}, NR^{c′}, NR^{c′}C(O)NR^{d′}, S(O), S(O)NR^{c′}, S(O)₂, or S(O)₂NR^{c′}; Q is H, halo, CN, NO₂, C₁₋₈ alkyl, C₂₋₈ alkenyl, C₂₋₈ alkynyl, C₁₋₈ haloalkyl, halosulfanyl, aryl, cycloalkyl, heteroaryl, or heterocycloalkyl, wherein said C₁₋₈ alkyl, C₂₋₈ alkenyl, C₂₋₈ alkynyl, C₁₋₈ haloalkyl, aryl, cycloalkyl, heteroaryl, or heterocycloalkyl is optionally substituted with 1, 2, 3 or 4 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy², CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{b′}, NR^{c′}C(O)NR^{c′}R^{d′}, NR^{c′}C(O)OR^{a′}, S(O)R^{b′}, S(O)NR^{c′}R^{d′}, S(O)₂R^{b′}, NR^{c′}S(O)₂R^{b′}, and S(O)₂NR^{c′}R^{d′}, Cy¹ and Cy² are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, OR^{a″}, S^{a″}, C(O)R^{b″}, C(O)NR^{c″}R^{d″}, C(O)OR^{a″}, OC(O)R^{b″}, OC(O)NR^{c″}R^{d″}, NR^{c″}R^{d″}, NR^{c″}C(O)R^{d″}, NR^{c″}C(O)OR^{a″}, NR^{c″}S(O)R^{b″}, NR^{c″}S(O)₂R^{b″}, S(O)R^{b″}, S(O)NR^{c″}R^{d″}, S(O)₂R^{b″}, and S(O)₂NR^{c″}R^{d″}; R¹, R², R³, and R⁴ are independently selected from H, halo, C₁₋₄ alkyl, C₂₋₄ alkenyl, C₂₋₄ alkynyl, C₁₋₄ haloalkyl, halosulfanyl, aryl, cycloalkyl, heteroaryl, heterocycloalkyl, CN, NO₂, OR⁷, SR⁷, C(O)R⁸, C(O)NR⁹R¹⁰, C(O)OR⁷OC(O)R⁸, OC(O)NR⁹R¹⁰, NR⁹R¹⁰, NR⁹C(O)R⁸, NR^{c}C(O)OR⁷, S(O)R⁸, S(O)NR⁹R¹⁰, S(O)₂R⁸, NR⁹S(O)₂R⁸, and S(O)₂NR⁹R¹⁰; R⁵ is H, halo, C₁₋₄ alkyl, C₂₋₄ alkenyl, C₂₋₄ alkynyl, C₁₋₄ haloalkyl, halosulfanyl, CN, NO₂, OR⁷, SR⁷, C(O)R⁸, C(O)NR⁹R¹⁰, C(O)OR⁷, OC(O)R⁸, OC(O)NR⁹R¹⁰, NR⁹R¹⁰, NR⁹C(O)R⁸, NR⁹C(O)OR⁷, S(O)R⁸, S(O)NR⁹R¹⁰, S(O)₂R⁸, NR⁹S(O)₂R⁸, or S(O)₂NR⁹R¹⁰; R⁶ is H, C₁₋₄ alkyl, C₂₋₄ alkenyl, C₂₋₄ alkynyl, C₁₋₄ haloalkyl, OR⁷, C(O)R⁸, C(O)NR⁹R¹⁰, C(O)OR⁷, S(O)R⁸, S(O)NR⁹R¹⁰, S(O)₂Rx, or S(O)₂NR⁹R¹⁰; R⁷ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl; Rx is H, C₁₋₆alkyl, C₁₋₆ haloalkyl, C₂₋₆ alkenyl, C₂₋₆alkynyl, aryl, cycloalkyl, heteroaryl, heterocycloalkyl, arylalkyl, heteroarylalkyl, cycloalkylalkyl or heterocycloalkylalkyl; R⁹ and R¹⁰ are independently selected from H, C₁₋₁₀ alkyl, C₁₋₆ haloalkyl, C₂₋₆ alkenyl, C₂₋₆ alkynyl, C₂₋₆ alkylcarbonyl, arylcarbonyl, C₁₋₆ alkylsulfonyl, arylsulfonyl, aryl, heteroaryl, cycloalkyl, heterocycloalkyl, arylalkyl, heteroarylalkyl, cycloalkylalkyl and heterocycloalkylalkyl; or R⁹ and R¹⁰ together with the N atom to which they are attached form a 4-, 5-, 6- or 7-membered heterocycloalkyl group; R¹¹ and R¹² are independently selected from H and -E¹-E²-E³-E⁴, D¹ and E¹ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, C₁₋₆alkoxy, C₁₋₆ haloalkoxy, amino, C₁₋₆ alkylamino, and C₂₋₈ dialkylamino; D² and E² are independently absent or independently selected from C₁₋₆ alkylene, C₂₋₆ alkenylene, C₂₋₆ alkynylene, (C₁₋₆ alkylene)ᵣ-O—(C₁₋₆ alkylene)ₛ. (C₁₋₆ alkylene)ᵣ-S—(C₁₋₆ alkylene)ₛ, (C₁₋₆ alkylene)ᵣ-NR^{c}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{c}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{c}—(C₁₋₆ alkylene), and (C₁₋₆ alkylene)ᵣ-NR^{c}CONR^{f}—(C₁₋₆ alkylene)ₛ, wherein each of the C₁₋₆ alkylene, C₂₋₆ alkenylene, and C₂₋₆ alkynylene is optionally substituted by 1, 2 or 3 substituents independently selected from halo. CN, NO₂, N₃, SCN, OH, C₁₋₆ alkyl, C₁₋₆ haloalkyl, C₂₋₈alkoxyalkyl, C₁₋₆ alkoxy, C₁₋₆ haloalkoxy, amino, C₁₋₆ alkylamino, and C₂₋₈ dialkylamino; D³ and E³ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino; D⁴ and E⁴ are independently selected from H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆alkyl)R^{b}, and S(O)₂NR^{c}R^{d}, wherein said C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆ alkyl))R^{b}, and S(O)₂NR^{c}R^{d}; R^{a} is H, Cy¹, —(C₁₋₆ alkyl)-Cy¹, C₁₋₆ alkyl, C₁₋₆ haloalkyl, C₂₋₆ alkenyl, C₂₋₆ alkynyl, wherein said C₁₋₆alkyl, C₁₋₆ haloalkyl, C₂₋₆alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl; R^{b} is H, Cy¹, —(C₁₋₆ alkyl)-Cy¹, C₁₋₆ alkyl, C₁₋₆ haloalkyl, C₂₋₆ alkenyl, C₂₋₆ alkynyl, wherein said C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, C₁₋₆ haloalkyl, halosulfanyl, aryl, arylalkyl, heteroaryl, heteroarylalkyl, cycloalkyl and heterocycloalkyl: R^{a′} and R^{a″} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆alkyl, C₁₋₆ haloalkyl. C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo. C₁₋₆ alkyl, C₁₋₆ haloalkyl, halosulfanyl, aryl, arylalkyl, heteroaryl, heteroarylalkyl, cycloalkyl and heterocycloalkyl, R^{b′} and R^{b″} are independently selected from H, C₁₋₆alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆ alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆ haloalkyl, C₁₋₆haloalkyl, halosulfanyl, aryl, arylalkyl, heteroaryl, heteroarylalkyl, cycloalkyl and heterocycloalkyl; R^{c} and R^{d} are independently selected from H, Cy¹, —(C₁₋₆ alkyl)-Cy¹, C₁₋₁₀ alkyl, C₁₋₆ haloalkyl, C₂₋₆ alkenyl, C₂₋₆ alkynyl, wherein said C₁₋₁₀ alkyl, C₁₋₆ haloalkyl, C₂₋₆ alkenyl, or C₂₋₆ alkynyl, is optionally substituted with 1, 2, or 3 substituents independently selected from Cy¹, —(C₁₋₆ alkyl)-Cy¹, OH, CN, amino, halo, C₁₋₆ alkyl, C₁₋₆ haloalkyl, C₁₋₆ haloalkyl, and halosulfanyl; or R^{c} and R^{d} together with the N atom to which they are attached form a 4-, 5-, 6- or 7-membered heterocycloalkyl group optionally substituted with 1, 2, or 3 substituents independently selected from Cy¹, —(C₁₋₆alkyl)-Cyr, OH, CN, amino, halo, C₁₋₆alkyl, C₁₋₆ haloalkyl, C₁₋₆ haloalkyl, and halosulfanyl; R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆ haloalkyl, C₁₋₆ haloalkyl, halosulfanyl, aryl, arylalkyl, heteroaryl, heteroarylalkyl, cycloalkyl and heterocycloalkyl; or R^{c′} and R^{d′} together with the N atom to which they are attached form a 4-, 5-, 6- or 7-membered heterocycloalkyl group optionally substituted with 1, 2, or 3 substituents independently selected from OH, CN, amino, halo, C₁₋₆ alkyl, C₁₋₆ haloalkyl, C₁₋₆ haloalkyl, halosulfanyl, aryl, arylalkyl, heteroaryl, heteroarylalkyl, cycloalkyl and heterocycloalkyl; R^{c″} and R^{d″} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, halosulfanyl, C₁₋₆ haloalkyl, aryl, arylalkyl, heteroaryl, heteroarylalkyl, cycloalkyl and heterocycloalkyl; or R^{c″} and R^{d″} together with the N atom to which they are attached form a 4-, 5-, 6- or 7-membered heterocycloalkyl group optionally substituted with 1, 2, or 3 substituents independently selected from OH. CN, amino, halo, C₁₋₄alkyl, C₁₋₆ haloalkyl, C₁₋₆ haloalkyl, halosulfanyl, aryl, arylalkyl, heteroaryl, heteroarylalkyl, cycloalkyl and heterocycloalkyl; Rⁱ is H, CN, NO₂, or C₁₋₆ alkyl; R^{e} and R^{f} are independently selected from H and C₁₋₆ alkyl; Rⁱ is H, CN, or NO₂; m is 0 or 1; n is 0 or 1; p is 0, 1, 2, 3, 4, 5, or 6; q is 0, 1, 2, 3, 4, 5 or 6: r is 0 or 1: and s is 0 or 1.

In some embodiments, the mitochondrial lipid shunt modulator is a Janus kinase inhibitor compound. In some embodiments, the mitochondrial lipid shunt modulator is ruxolitinib or a pharmaceutically acceptable salt thereof. In some embodiments, the compound is

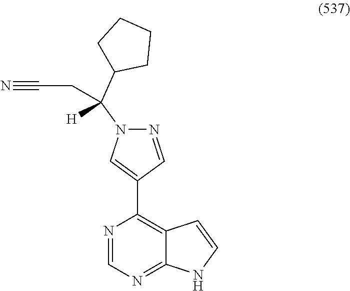 [SMILES: N#CC[C@H](C1CCCC1)n1cc(-c2ncnc3[nH]ccc23)cn1]

or a pharmaceutically acceptable salt thereof.

In some embodiments, the mitochondrial lipid shunt modulator is a transforming growth factor-β or activin receptor-like kinase inhibitor compound. In some embodiments, the mitochondrial lipid shunt modulator is a compound disclosed in U.S. Pat. No. 6,465,493, or a salt or derivative thereof, which is incorporated herein by reference in its entirety. In some embodiments, the mitochondrial lipid shunt modulator is a compound of Formula (XXV):

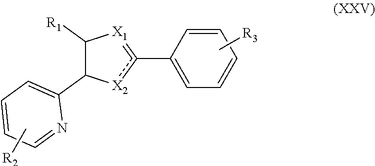

or a pharmaceutically acceptable salt thereof, wherein: wherein R₁ is naphthyl, anthracenyl, or phenyl optionally substituted with one or more substituents selected from the group consisting of halo, C₁₋₆alkoxy, C₁₋₆alkylthio, C₁₋₆alkyl, —O—(CH₂)ₙ-Ph, —S—(CH₂)ₙ-Ph, cyano, phenyl, and CO₂R, wherein R is hydrogen or C₁₋₆alkyl and n is 0, 1, 2 or 3; or Rⱼ is phenyl fused with an aromatic or non-aromatic cyclic ring of 5-7 members wherein said cyclic ring optionally contains up to two heteroatoms, independently selected from N, O and S; R₂ is H, NH(CH₂)ₙ-Ph or NH—C₁₋₆alkyl, wherein n is 0, 1, 2 or 3; R₃ is CO₂H, CONH₂, CN, NO₂, C₁₋₆alkylthio, —SO₂—C₁₋₆alkyl, C₁₋₆alkoxy, SONH₂, CONHOH, NH₂, CHO, CH₂OH, CH₂NH₂, or CO₂R, wherein R is hydrogen or C₁₋₆alkyl; and one of X₁ and X₂ is N or CR′, and the other is NR′ or CHR′ wherein R′ is hydrogen, OH, C₁₋₆ alkyl, or C₃₋₇cycloalkyl; or when one of X₁ and X₂ is N or CR′ then the other may be S or O.

In some embodiments, the mitochondrial lipid shunt modulator is a transforming growth factor-β or activin receptor-like kinase inhibitor compound. In some embodiments, the mitochondrial lipid shunt modulator is SB-505124 or a pharmaceutically acceptable salt thereof. In some embodiments, the compound is

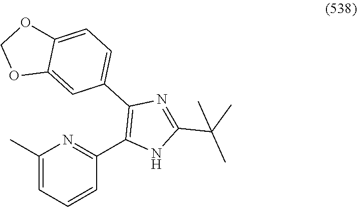 [SMILES: Cc1cccc(-c2[nH]c(C(C)(C)C)nc2-c2ccc3c(c2)OCO3)n1]

or a pharmaceutically acceptable salt thereof. In some embodiments, the mitochondrial lipid shunt modulator is SB-431524 or a pharmaceutically acceptable salt thereof. In some embodiments, the compound is

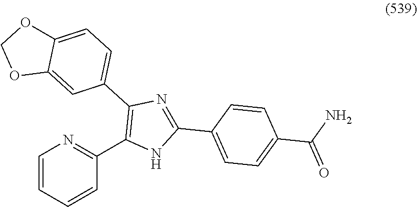 [SMILES: NC(=O)c1ccc(-c2nc(-c3ccc4c(c3)OCO4)c(-c3ccccn3)[nH]2)cc1]

or a pharmaceutically acceptable salt thereof.

In some embodiments, the mitochondrial lipid shunt modulator is a p38 MAPK inhibitor compound. In some embodiments, the mitochondrial lipid shunt modulator is SB-203580 or a pharmaceutically acceptable salt thereof. In some embodiments, the compound is

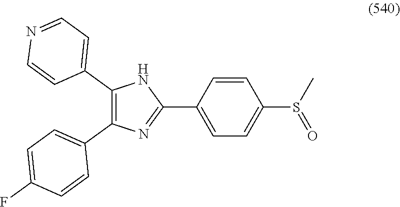 [SMILES: CS(=O)c1ccc(-c2nc(-c3ccc(F)cc3)c(-c3ccncc3)[nH]2)cc1]

or a pharmaceutically acceptable salt thereof.

In some embodiments, the mitochondrial lipid shunt modulator is an anthraquinone derivative compound. In some embodiments, the mitochondrial lipid shunt modulator is emodin or a pharmaceutically acceptable salt thereof. In some embodiments, the compound is

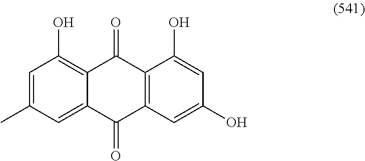 [SMILES: Cc1cc(O)c2c(c1)C(=O)c1cc(O)cc(O)c1C2=O]

or a pharmaceutically acceptable salt thereof.

In some embodiments, the mitochondrial lipid shunt modulator is a calcium/calmodulin-dependent protein kinase II inhibitor compound. In some embodiments, the mitochondrial lipid shunt modulator is KN-93 or a pharmaceutically acceptable salt thereof. In some embodiments, the compound is

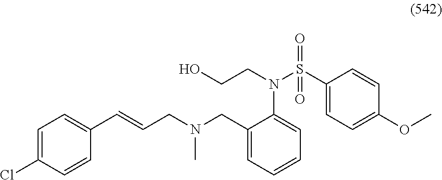 [SMILES: COc1ccc(S(=O)(=O)N(CCO)c2ccccc2CN(C)C/C=C/c2ccc(Cl)cc2)cc1]

or a pharmaceutically acceptable salt thereof.

In some embodiments, the mitochondrial lipid shunt modulator is a protein kinase C inhibitor compound. In some embodiments, the mitochondrial lipid shunt modulator is bisindolylmaleimide IX or a pharmaceutically acceptable salt thereof. In some embodiments, the compound is

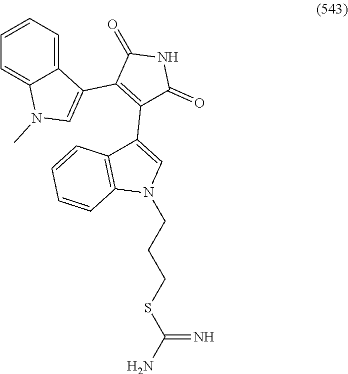 [SMILES: Cn1cc(C2=C(c3cn(CCCSC(=N)N)c4ccccc34)C(=O)NC2=O)c2ccccc21]

or a pharmaceutically acceptable salt thereof.

In some embodiments, the mitochondrial lipid shunt modulator is a type-I transforming growth factor beta inhibitor compound. In some embodiments, the mitochondrial lipid shunt modulator is LY364947 or a pharmaceutically acceptable salt thereof. In some embodiments, the compound is

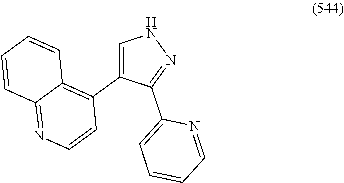 [SMILES: c1ccc(-c2n[nH]cc2-c2ccnc3ccccc23)nc1]

or a pharmaceutically acceptable salt thereof. In some embodiments, the mitochondrial lipid shunt modulator is PD169316 or a pharmaceutically acceptable salt thereof. In some embodiments, the compound is

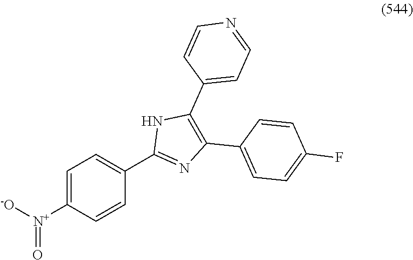

or a pharmaceutically acceptable salt thereof.

In some embodiments, the mitochondrial lipid shunt modulator is an autophagy inhibitor compound. In some embodiments, the mitochondrial lipid shunt modulator is LY294002 or a pharmaceutically acceptable salt thereof. In some embodiments, the compound is

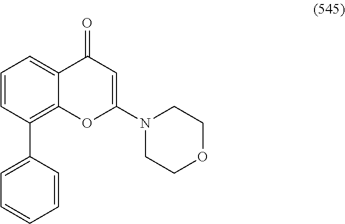 [SMILES: O=c1cc(N2CCOCC2)oc2c(-c3ccccc3)cccc12]

or a pharmaceutically acceptable salt thereof.

In some embodiments, the mitochondrial lipid shunt modulator is a MEK1/2 kinase inhibitor compound. In some embodiments, the mitochondrial lipid shunt modulator is U0126 or a pharmaceutically acceptable salt thereof. In some embodiments, the compound is

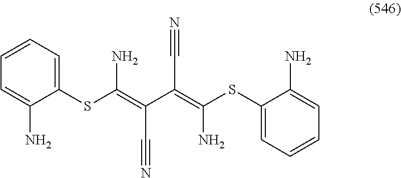 [SMILES: N#CC(=C(/N)Sc1ccccc1N)/C(C#N)=C(\N)Sc1ccccc1N]

or a pharmaceutically acceptable salt thereof. In some embodiments, the mitochondrial lipid shunt modulator is AS-703026, pimasertib, or a pharmaceutically acceptable salt thereof. In some embodiments, the compound is

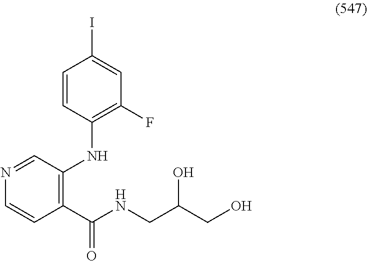 [SMILES: O=C(NCC(O)CO)c1ccncc1Nc1ccc(I)cc1F]

or a pharmaceutically acceptable salt thereof.

In some embodiments, the mitochondrial lipid shunt modulator is a PI3K p110β inhibitor compound. In some embodiments, the mitochondrial lipid shunt modulator is TGX-221 or a pharmaceutically acceptable salt thereof. In some embodiments, the compound is

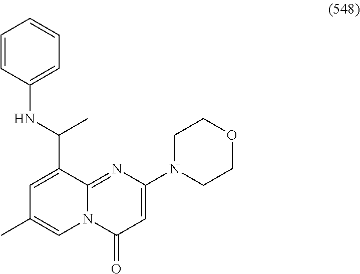 [SMILES: Cc1cc(C(C)Nc2ccccc2)c2nc(N3CCOCC3)cc(=O)n2c1]

or a pharmaceutically acceptable salt thereof.

In some embodiments, the mitochondrial lipid shunt modulator is a diterpene amine compound. In some embodiments, the mitochondrial lipid shunt modulator is leelamine or a pharmaceutically acceptable salt thereof. In some embodiments, the compound is

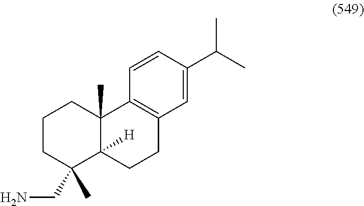

or a pharmaceutically acceptable salt thereof.

In some embodiments, the mitochondrial lipid shunt modulator is a sphingosine kinase inhibitor compound. In some embodiments, the mitochondrial lipid shunt modulator is sphingosine inhibitor compound or a pharmaceutically acceptable salt thereof. In some embodiments, the compound is

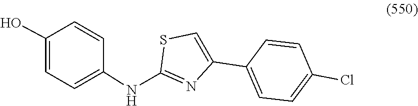 [SMILES: Oc1ccc(Nc2nc(-c3ccc(Cl)cc3)cs2)cc1]

or a pharmaceutically acceptable salt thereof.

In some embodiments, the mitochondrial lipid shunt modulator is a stilbene compound. In some embodiments, the mitochondrial lipid shunt modulator is piceatannol or a pharmaceutically acceptable salt thereof. In some embodiments, the compound is

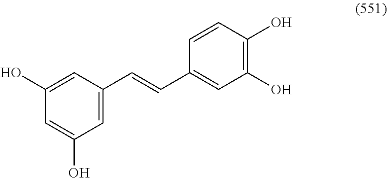 [SMILES: Oc1cc(O)cc(/C=C/c2ccc(O)c(O)c2)c1]

or a pharmaceutically acceptable salt thereof.

In some embodiments, the mitochondrial lipid shunt modulator is a cyclin-dependent kinase inhibitor compound. In some embodiments, the mitochondrial lipid shunt modulator is roscovitine or a pharmaceutically acceptable salt thereof. In some embodiments, the compound is

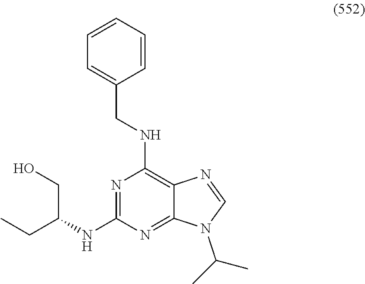 [SMILES: CC[C@H](CO)Nc1nc(NCc2ccccc2)c2ncn(C(C)C)c2n1]

or a pharmaceutically acceptable salt thereof.

In some embodiments, the mitochondrial lipid shunt modulator is a kinase inhibitor compound. In some embodiments, the mitochondrial lipid shunt modulator is sorafenib, BAY 43-9006, or a pharmaceutically acceptable salt thereof. In some embodiments, the compound is

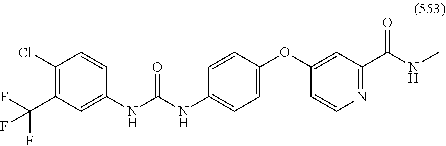 [SMILES: CNC(=O)c1cc(Oc2ccc(NC(=O)Nc3ccc(Cl)c(C(F)(F)F)c3)cc2)ccn1]

or a pharmaceutically acceptable salt thereof.

In some embodiments, the mitochondrial lipid shunt modulator is a PI3Kγ inhibitor inhibitor compound. In some embodiments, the mitochondrial lipid shunt modulator is a compound disclosed in U.S. Pat. No. 7,846,925, or a salt or derivative thereof, which is incorporated herein by reference in its entirety. In some embodiments, the mitochondrial lipid shunt modulator is a compound of Formula (XXVI):

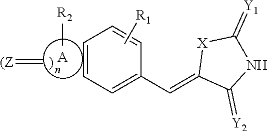

(XXVI) or a pharmaceutically acceptable salt thereof, wherein: A is a 5-8 membered heterocyclic group;
X is S or O, Y¹ and Y² are independently S or O; Z is S or O, R′ is H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁—C(-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino or carbamate; R² is selected from the group consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, C₁-C₆-alkyl carbamate, sulfonylamino, sulfanyl, or sulfonyl, n is 0, 1 or 2.

In some embodiments, the mitochondrial lipid shunt modulator is a PI3Kγ inhibitor compound. In some embodiments, the mitochondrial lipid shunt modulator is AS-604850, or a pharmaceutically acceptable salt thereof. In some embodiments, the compound is

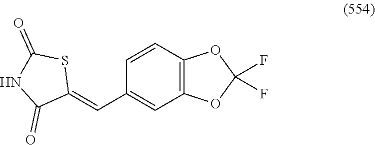 [SMILES: O=C1NC(=O)/C(=C/c2ccc3c(c2)OC(F)(F)O3)S1]

or a pharmaceutically acceptable salt thereof.

In some embodiments, the mitochondrial lipid shunt modulator is a cannabinoid receptor antagonist compound. In some embodiments, the mitochondrial lipid shunt modulator is O-1918, or a pharmaceutically acceptable salt thereof. In some embodiments, the compound is

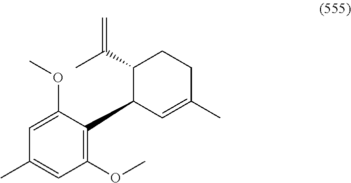

or a pharmaceutically acceptable salt thereof.

In some embodiments, the mitochondrial lipid shunt modulator is a tyrosine kinase inhibitor compound. In some embodiments, the mitochondrial lipid shunt modulator is PD 166326, or a pharmaceutically acceptable salt thereof. In some embodiments, the compound is

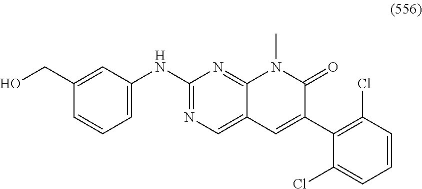 [SMILES: Cn1c(=O)c(-c2c(Cl)cccc2Cl)cc2cnc(Nc3cccc(CO)c3)nc21]

or a pharmaceutically acceptable salt thereof.

In some embodiments, the mitochondrial lipid shunt modulator is a casein kinase 1 inhibitor compound. In some embodiments, the mitochondrial lipid shunt modulator is D4476, or a pharmaceutically acceptable salt thereof. In some embodiments, the compound is

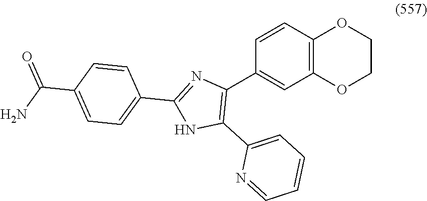 [SMILES: NC(=O)c1ccc(-c2nc(-c3ccc4c(c3)OCCO4)c(-c3ccccn3)[nH]2)cc1]

or a pharmaceutically acceptable salt thereof.

In some embodiments, the mitochondrial lipid shunt modulator is a PIM1 kinase inhibitor compound. In some embodiments, the mitochondrial lipid shunt modulator is a compound disclosed in U.S. Pat. No. 8,637,558, or a salt or derivative thereof, which is incorporated herein by reference in its entirety. In some embodiments, the mitochondrial lipid shunt modulator is a compound of Formula (XXVII):

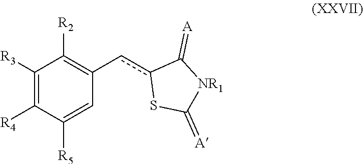

or a pharmaceutically acceptable salt thereof, wherein:

Wherein, A and A′ are each independently O or S; R¹ is hydrogen or CH₂CH₂OH; R₂, R₃, R₄ and R₅ may be each independently selected from hydrogen, nitro, amine, alkoxy, alkyl, trifluoromethyl, carboxyl, halogen or

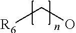

where said

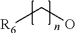

may bind to the C3- or C4-position of the benzene ring; R₆ may be selected from the group consisting of hydrogen, methyl, ethyl, unsubstituted or substituted (hetero)cycloalkyl, (hetero)cycloalkenyl or (hetero)aryl: the dotted line indicates a single or a double bond; and n may be an integer from 0 to 5.

Further, in the above formula, in case that R₃ is

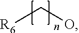

R₂, R₄ and R₅ may be each independently selected from the group consisting of H, NO₂, NH₂, CH₃, Cl, Br, F, COOH, CF₃, CH₃O and CH₃CH₂O; and in the case that R₄ is

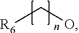

R₂, R₃ and R₅ may be each independently selected from the group consisting of H, NO₂, NH₂, CH₃, Cl, Br, F, COOH, CF₃, CH₃O and CH₃CH₂O.

In some embodiments, the mitochondrial lipid shunt modulator is a PIM1 kinase inhibitor compound. In some embodiments, the mitochondrial lipid shunt modulator is SMI-4a, or a pharmaceutically acceptable salt thereof. In some embodiments, the compound is

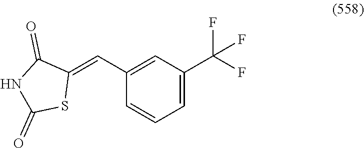 [SMILES: O=C1NC(=O)/C(=C/c2cccc(C(F)(F)F)c2)S1]

or a pharmaceutically acceptable salt thereof.

In some embodiments, the mitochondrial lipid shunt modulator is a PI3K p110α inhibitor compound. In some embodiments, the mitochondrial lipid shunt modulator is PI3-Kinase alpha Inhibitor 2, or a pharmaceutically acceptable salt thereof. In some embodiments, the compound

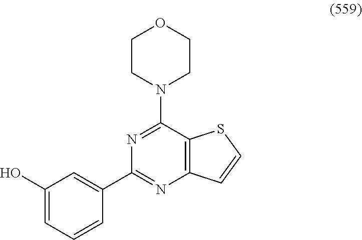 [SMILES: Oc1cccc(-c2nc(N3CCOCC3)c3sccc3n2)c1]

or a pharmaceutically acceptable salt thereof.

In some embodiments, the mitochondrial lipid shunt modulator is a Akt inhibitor compound. In some embodiments, the mitochondrial lipid shunt modulator is ML-9, or a pharmaceutically acceptable salt thereof. In some embodiments, the compound is

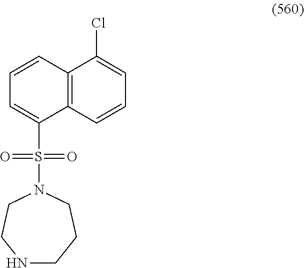 [SMILES: O=S(=O)(c1cccc2c(Cl)cccc12)N1CCCNCC1]

or a pharmaceutically acceptable salt thereof.

In some embodiments, the mitochondrial lipid shunt modulator is a tau-protein kinase inhibitor compound. In some embodiments, the mitochondrial lipid shunt modulator is SB-415286 or a pharmaceutically acceptable salt thereof. In some embodiments, the compound is

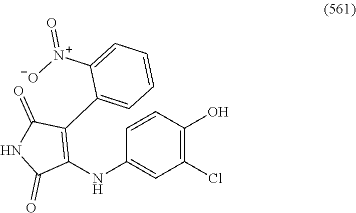 [SMILES: O=C1NC(=O)C(c2ccccc2[N+](=O)[O-])=C1Nc1ccc(O)c(Cl)c1]

or a pharmaceutically acceptable salt thereof.

In some embodiments, the mitochondrial lipid shunt modulator is a tyrosine kinase antagonist compound. In some embodiments, the mitochondrial lipid shunt modulator is AG-17 or a pharmaceutically acceptable salt thereof. In some embodiments, the compound is

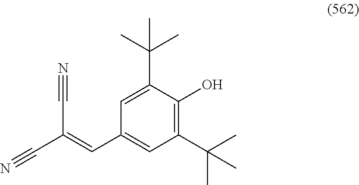 [SMILES: CC(C)(C)c1cc(C=C(C#N)C#N)cc(C(C)(C)C)c1O]

or a pharmaceutically acceptable salt thereof.

In some embodiments, the mitochondrial lipid shunt modulator is an EGF receptor kinase inhibitor compound. In some embodiments, the mitochondrial lipid shunt modulator is RG-13022 or a pharmaceutically acceptable salt thereof. In some embodiments, the compound is

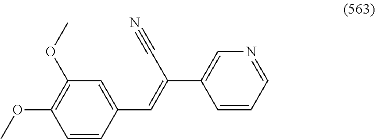 [SMILES: COc1ccc(/C=C(\C#N)c2cccnc2)cc1OC]

or a pharmaceutically acceptable salt thereof.

In some embodiments, the mitochondrial lipid shunt modulator is a MAPKα inhibitor compound. In some embodiments, the mitochondrial lipid shunt modulator is SB-202190 or a pharmaceutically acceptable salt thereof. In some embodiments, the compound is

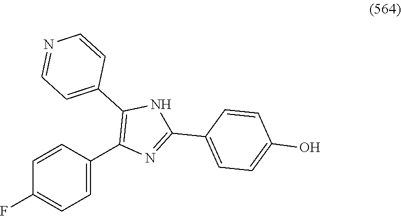 [SMILES: Oc1ccc(-c2nc(-c3ccc(F)cc3)c(-c3ccncc3)[nH]2)cc1]

or a pharmaceutically acceptable salt thereof.

In some embodiments, the mitochondrial lipid shunt modulator is a Her2/Neu Tyrosine kinase inhibitor compound. In some embodiments, the mitochondrial lipid shunt modulator is AG-825 or a pharmaceutically acceptable salt thereof. In some embodiments, the compound is

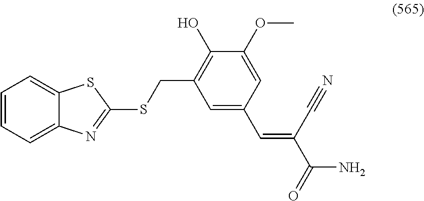 [SMILES: COc1cc(/C=C(\C#N)C(N)=O)cc(CSc2nc3ccccc3s2)c1O]

or a pharmaceutically acceptable salt thereof.

In some embodiments, the mitochondrial lipid shunt modulator is a CSAID kinase inhibitor compound. In some embodiments, the mitochondrial lipid shunt modulator is CAY 10571 or a pharmaceutically acceptable salt thereof. In some embodiments, the compound is

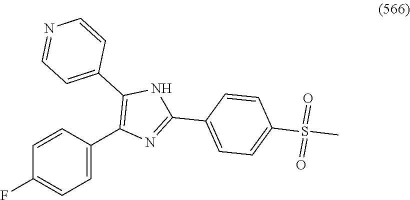 [SMILES: CS(=O)(=O)c1ccc(-c2nc(-c3ccc(F)cc3)c(-c3ccncc3)[nH]2)cc1]

or a pharmaceutically acceptable salt thereof.

In some embodiments, the mitochondrial lipid shunt modulator is an apoptosis blocker. In some embodiments, the mitochondrial lipid shunt modulator is a compound disclosed in van Delft et al. (Nature Chemical Biology (2019) 15(11): 1057-1066 or a salt or derivative thereof, which is incorporated herein by reference in its entirety. In some embodiments, the mitochondrial lipid shunt modulator is WEHI-9625 or a pharmaceutically acceptable salt thereof.

In some embodiments, the compound is selected from the group comprising:

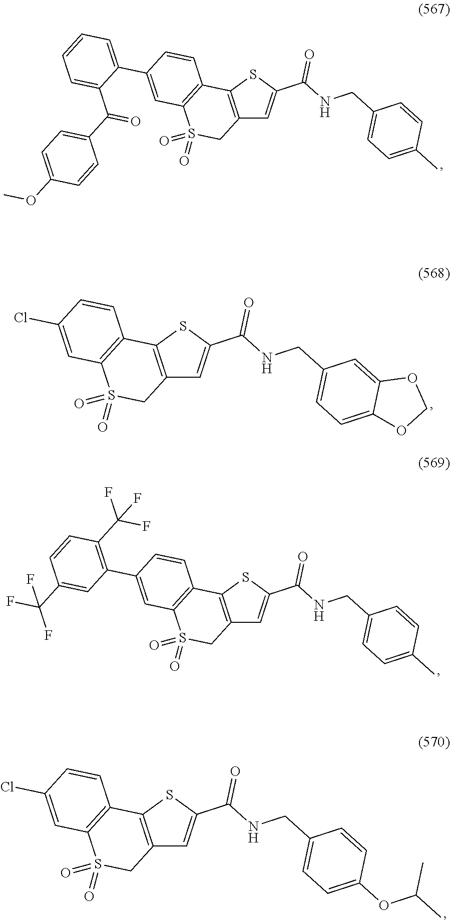

or a pharmaceutically acceptable salt thereof. In some embodiments, the compound is selected from the group comprising:

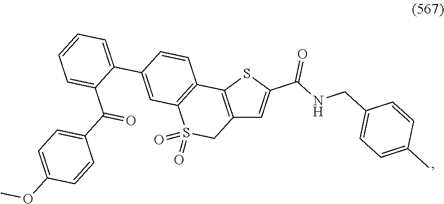

or a pharmaceutically acceptable salt thereof.

In some embodiments, the mitochondrial lipid shunt modulator is a Rho kinase inhibitor compound. In some embodiments, the mitochondrial lipid shunt modulator is a compound disclosed in U.S. Pat. No. 6,153,608 or a salt or derivative thereof, which is incorporated herein by reference in its entirety. In some embodiments, the mitochondrial lipid shunt modulator is a compound of Formula (XXVIII):

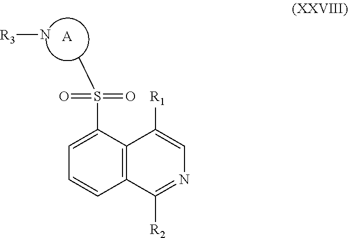

or a pharmaceutically acceptable salt thereof, wherein:
R¹ is alkyl, alkenyl, alkynyl, alkoxy, hydroxy, cyano, or halogen; R² is hydrogen, hydroxy, or halogen; R³ is hydrogen, alkyl, or amidino; and Ring A is a 5 to 11-membered cyclic amino group which may be bridged between two carbon atoms in optional positions and which may be substituted, or a pharmaceutically acceptable salt, hydrate or solvate thereof.

In some embodiments, the mitochondrial lipid shunt modulator is a Rho-associated kinase inhibitor compound. In some embodiments, the mitochondrial lipid shunt modulator is (S)-H-1152 or a pharmaceutically acceptable salt thereof. In some embodiments, the compound

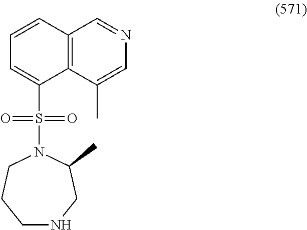 [SMILES: Cc1cncc2cccc(S(=O)(=O)N3CCCNC[C@@H]3C)c12]

or a pharmaceutically acceptable salt thereof.

In some embodiments, the mitochondrial lipid shunt modulator is a transforming growth factor-β or activin receptor-like kinase inhibitor compound. In some embodiments, the mitochondrial lipid shunt modulator is a compound disclosed in U.S. Pat. No. 6,403,588, or a salt or derivative thereof, which is incorporated herein by reference in its entirety. In some embodiments, the mitochondrial lipid shunt modulator is a compound of Formula (XXIX):

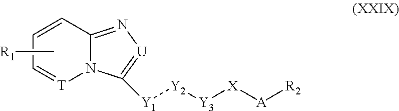

or a pharmaceutically acceptable salt thereof, wherein: wherein R¹ represents —H, -a lower alkyl, -a lower alkenyl, lower alkyl, -a cycloalkyl, -a cycloalkenyl, -a halogen, —NO₂, —CN, -a halogenated lower alkyl, —OR^{a}, —SR^{a}, —SO₂R^{a}, —SOR^{a}, —CO₂R^{a}, —CO—R^{a}, -an aryl, -a lower alkylene-an aryl, —O-a lower alkylene-an aryl, —CONR^{a}R^{b}—, —CO-(a nitrogen-containing saturated heterocyclic group which may be substituted by a lower alkyl group), —SO₂NR^{a}R^{b}, —SO₂— (a nitrogen-containing saturated heterocyclic group which may be substituted by a lower alkyl group), —SO₃H, -(a nitrogen-containing saturated heterocyclic group which may be substituted by a lower alkyl group), —NR^{c}R^{b}, —CONR^{a}-a lower alcylene-OR^{b}, —CONR^{a}-a lower alkylene-NR^{b}R^{c}, —CONR^{a}-a lower alkylene-(a nitrogen-containing saturated heterocyclic group which may be substituted by a lower alkyl group), —O-a lower alkylene-OR^{a}, —O-a lower alkylene-O-a lower alkylene-OR^{a}, —O-a lower alkylene-NR^{a}R^{b}, —O-a lower alkylene-(a nitrogen-containing saturated heterocyclic group which may be substituted by a lower alkyl group), —O-a lower alkylene-O-a lower alkylene-NRR^{b}, —O-a lower alkylene-O-a lower alkylene-(a nitrogen-containing saturated heterocyclic group which may be substituted by a lower alkyl group), —O-a lower alkylene-NR-a lower alkylene-NR^{a}R^{b}, —O-a lower alkylene-NR^{C}-a lower alkylene-(a nitrogen-containing saturated heterocyclic group which may be substituted by a lower alkyl group), —OCO—NR^{a}R^{b}, —OCO-(a nitrogen-containing saturated heterocyclic group which may be substituted by a lower alkyl group), —NR^{a}—SO₂R^{b}, —NR^{c}-a lower alkylene-NR^{a}R^{b}, —NR^{c}-a lower alkylene-(a nitrogen-containing saturated heterocyclic group which may be substituted by a lower alkyl group), —N(a lower alkylene-NR^{a}R^{b})₂, —N(a lower alkylene-(a nitrogen-containing saturated heterocyclic group which may be substituted by a lower alkyl group))₂, —CONR^{a}—OR^{b}, —NR^{a}—COR^{b}, —NR^{a}—CO—NR^{b}R^{c}, —NR^{a}—CO-(a nitrogen-containing saturated heterocyclic group which may be substituted by a lower alkyl group), or —OCOR^{a}; R^{a}, R^{b} and R^{c} which may be the same or different, represent —H, -a lower alkyl or -an aryl; T represents N or CR^{1a}, U represents N or CR³; N represents an integer, 1, 2 or 3; in Y¹ . . . Y² . . . Y³, . . . represents a single bond on one side and a single or double bond on the other side, Y¹ represents CR⁵ or CR^{5a}R^{5b}, Y² represents N, NH, CR^{4a} or CR^{4b}R^{4c}, and Y³ represents NR⁶, CR^{4d} or CR^{4e}R^{4f}, whereas Y³ represents NR⁶ when Y² represents CR^{4a} or CR^{4b}R^{4c}, X represents S, SO or SO₂; “A” represents a linkage, a lower alkylene, a lower alkenylene or a lower alkenylene; R² represents -a lower alkyl which may have one or more substituents, -a lower alkenyl which may have one or more substituents, -a lower alkynyl which may have one or more substituents, -a cycloalkyl which may have one or more substituents, -a cycloalkenyl which may have one or more substituents, —N═O, -an aryl which may have one or more substituents, or -a heteroaryl which may have one or more substituents; R^{1a}, R³, R^{4a}, R^{4b}, R^{4c}, R^{4d}, R^{4e}, R^{4f}, R^{5a}, and R^{5b}, which may be the same or different, represent a group defined by R¹, whereas R^{4b} and R^{4c}, R^{4c}, and R^{4f}, or R^{5a} and R^{5b} may be combined with each other to form an oxo group (═O); R_{and R}⁶ which may be the same or different, represent —H, -a lower alkyl which may have one or more substituents, -a lower alkenyl which may have one or more substituents, -a lower alkynyl which may have one or more substituents.

In some embodiments, the mitochondrial lipid shunt modulator is a PI3K kinase inhibitor compound. In some embodiments, the mitochondrial lipid shunt modulator is PIK-75 or a pharmaceutically acceptable salt thereof. In some embodiments, the compound is

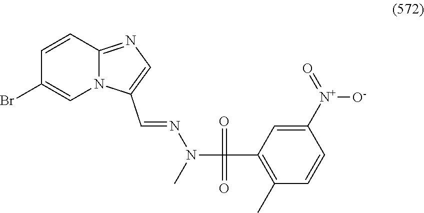

or a pharmaceutically acceptable salt thereof.

In some embodiments, the mitochondrial lipid shunt modulator is a ERK kinase inhibitor compound. In some embodiments, the mitochondrial lipid shunt modulator is CAY10561 or a pharmaceutically acceptable salt thereof. In some embodiments, the compound is

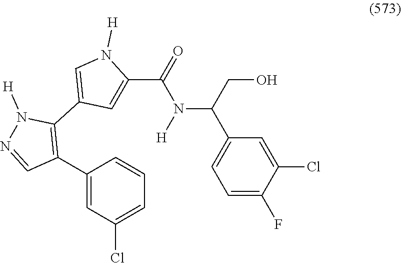 [SMILES: O=C(NC(CO)c1ccc(F)c(Cl)c1)c1cc(-c2[nH]ncc2-c2cccc(Cl)c2)c[nH]1]

or a pharmaceutically acceptable salt thereof.

### Methods of Use

The present disclosure provides methods for modulating the mitochondrial lipid shunt pathway, e.g., by administering to a cell or subject a mitochondrial lipid shunt modulator described herein. The present disclosure also provides methods for the treatment or prevention of a neurodegenerative disease, disorder or condition. Such methods comprise the step of administering to the subject in need thereof an effective amount of a mitochondrial lipid shunt modulator or a pharmaceutically acceptable salt, solvate, hydrate, tautomer, stereoisomer thereof, or a pharmaceutical composition thereof. In certain embodiments, the methods described herein include administering to a subject an effective amount of a mitochondrial lipid shunt modulator, or a pharmaceutically acceptable salt thereof, or a pharmaceutical composition thereof.

The methods described herein may be useful in the treatment or prevention of a neurodegenerative disease. Exemplary neurodegenerative diseases include amyotrophic lateral sclerosis, Alzheimer's disease, Huntington's chorea, Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Parkinson's disease, multiple system atrophy, spinal muscular atrophy (SMA), progressive spinobulbar muscular atrophy (e.g., Kennedy disease), spinocerebellar ataxia, pantothenate kinase-associated neurodegeneration (PANK), spinal degenerative disease/motor neuron degenerative diseases, upper motor neuron disorder, lower motor neuron disorder, Lytigo-bodig (amyotrophic lateral sclerosis-parkinsonism dementia), Guam-Parkinsonism dementia, hippocampal sclerosis, corticobasal degeneration, a demyelinating disease. All types of neurodegenerative diseases disclosed herein or known in the art are contemplated as being within the scope of the disclosure.

In an embodiment, the neurodegenerative disease is amyotrophic lateral sclerosis. ALS is an aggressive, multi-factorial, and polygenic disease characterized by rapid motor neuron degeneration and muscle wasting. Over 20 mutations have been linked to ALS, and the list is still expanding. Hence, although most ALS cases are thought to be sporadic, and increasing number is associated with a heritable genetic variation. Most cases of ALS, whether heritable or sporadic, display a cell biological pathology involving the TAR DNA-binding protein 43 (TDP43), thought to accompany stress granule dysfunction. One of the most prominent clinical features of ALS is the dysregulation of metabolic pathways. Increased energy expenditure, inefficient use of glucose, and over-reliance of fatty acid oxidation (FAO), sometimes referred to as hypermetabolism, are apparent in patients and disease models.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

In one aspect, the present disclosure further features a method for evaluating the stress response in a cell or a subject. The method provides a means for qualitatively or quantitatively evaluating the cellular response to introduction of a stressor. Exemplary stressors include oxidative stress, starvation, pH imbalance, temperature (e.g., thermal stress, cold conditions). In an embodiment, the method measures the formation of stress granules in a cell or subject in the presence of a stressor (e.g., a stressor described herein). In an embodiment, the method measures the formation of lipid droplets in a cell or subject in the presence of a stressor (e.g., a stressor described herein). In an embodiment, the method measures the formation of both stress granules and lipid droplets in a cell or subject in the presence of a stressor (e.g., a stressor described herein). In an embodiment, the method replicates ALS conditions, e.g., serve as a model for the study of ALS. In an embodiment, the cell is a stem cell, e.g., an induced pluripotent stem cell (iPSC). In an embodiment, the cell is a stem cell (e.g., an induced pluripotent stem cell) derived from an ALS patient.

The underlying causal biology for ALS development (etiology) is largely unknown. A major hurdle in the study of ALS has been the paucity of suitable cellular or animal models that faithfully recreate the disease state. For example, introducing causative genes into muse models may be of limited value, as mice simply do not develop symptoms that faithfully reflect the human disease. Moreover, even efforts to use advanced induced pluripotent stem cell (iPSC) technologies to generate “neuronal proxy” cells from actual ALS patient-derived cells have, so far, fallen short of replicating human disease. The lack of a cellular or animal model for ALS complicates the development of drug discovery campaigns against this disease, and frustrates the search for a cure.

iPSC technology may offer an extremely powerful way to generate human cellular tools for drug research in many disease areas by precisely-controlled ex vivo manipulation of easily and reproducibly procured cell samples from disease-inflicted human patients, thus bypassing the need for using onerous, invasive surgical methods to harvest the specific patient cell populations that are most useful for studying the disease. Therefore, the iPSC cell-technologies may be used to screen drug candidates or identify molecular biomarkers of the human disease in highly relevant human cells. However, such studies generally require that the iPSC derived cells exhibit a disease-correlated phenotype.

iPSC technologies may be applied to samples obtained from human ALS patients to generate neuronal proxy cells for drug discovery studies. Successfully generating the desired cell types requires high levels of technological expertise and expert application. However, when using this approach on ALS patient samples the resultant neuronal proxy cells that are generated appear devoid of any abnormal phenotype, especially a phenotype that appears to recapitulate or pre-empt human ALS patient disease pathophysiology.

The present disclosure relates to methods for the ex vivo manipulation of patient cells, in order to induce or express a human ALS disease-correlated phenotype, such that the resultant cells can be used to screen for effective ALS drugs and/or identify molecular biomarkers that can be used to diagnose disease and/or monitor disease progression and/or the effect of potential disease-modifying therapies.

iPSC derived neuronal proxy cells from ALS patients may not readily exhibit a disease-correlated phenotype unless appropriate disease-related cellular stimuli are applied. This disclosure features methods for the ex vivo stimulation of iPSC neuronal proxy cells, to induce a cellular phenotype that is predictive of the human disease. Such methods involve treating the neuronal proxy cells with metabolic or environmental stressors, carefully applied prior to and/or during drug screening or biomarker characterization.

Additional exemplary stressors can include, but are not limited to: short- or long-term nutrient starvation stress, ATP deprivation, thermal stress, oxidation stress and/or reactive oxygen species, known chemical stress inducers (e.g. arsenite exposure), elevated levels of small molecule products of fatty acid oxidation, or their metabolic derivatives (e.g. acetyl-Co or malonyl-CoA, respectively), stressful metabolic reducing agent “cocktails” (e.g. specific NAD, NADH, NADP, NADPH, FAD and FADH2 mixtures).

Measurement of Fatty Acid Oxidation Dependency in ALS-Patient Derived iPSCs

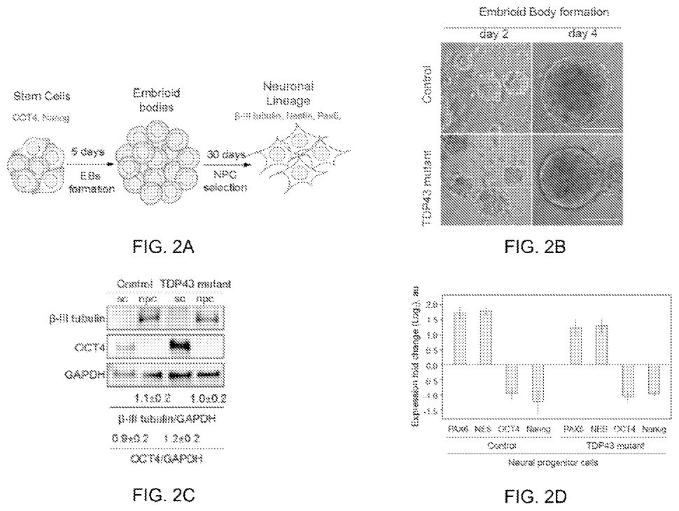

An exemplary method to assess the cellular stress response is shown in FIG. 2. As shown in FIGS. 2A-2C, iPSCs derived from an ALS patient with a mutation in TDP43 are differentiated along a neuronal lineage for 30 days and lineage factors are assessed by western blot and compared to healthy controls (FIGS. 2A-2C). At this point, the cells are challenged with a stressor from the group including, but not limited to, short- or long-term nutrient starvation stress, ATP deprivation, thermal stress, oxidation stress and/or reactive oxygen species, known chemical stress inducers (e.g. arsenite exposure), elevated levels of small molecule products of fatty acid oxidation, or their metabolic derivatives (e.g. acetyl-Co or malonyl-CoA, respectively), stressful metabolic reducing agent “cocktails” (e.g. specific NAD, NADH, NADP, NADPH, FAD and FADH2 mixtures). As shown in FIG. 2D, the fold change in oxygen consumption rate (OCR) for FAO is increased in TDP43 mutant cells, compared to controls, over the course of a six-hour nutrient starvation stress.

### Pharmaceutical Compositions, Kits, and Administration

The present invention provides pharmaceutical compositions comprising a mitochondrial lipid shunt modulator, e.g., as described herein, or a pharmaceutically acceptable salt, solvate, hydrate, tautomer, or stereoisomer, as described herein, and optionally a pharmaceutically acceptable excipient. In certain embodiments, the pharmaceutical composition described herein comprises a mitochondrial lipid shunt modulator, e.g., as described herein, or a pharmaceutically acceptable salt thereof, and optionally a pharmaceutically acceptable excipient. In certain embodiments, the mitochondrial lipid shunt modulator, e.g., as described herein, or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the mitochondrial lipid shunt modulator, e.g., as described herein, (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a mitochondrial lipid shunt modulator, e.g., as described herein, for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the mitochondrial lipid shunt modulators, e.g., as described herein, may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The mitochondrial lipid shunt modulator, e.g., as described herein, and compositions thereof can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, anti-apoptitic agents and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the disclosure are kits (e.g., pharmaceutical packs). The inventive kits may be useful for preventing and/or treating a neurodegenerative disease such as ALS, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a mitochondrial lipid shunt modulator, e.g., as described herein. In some embodiments, the mitochondrial lipid shunt modulator, e.g., as described herein, provided in the container and the second container are combined to form one-unit dosage form.

Thus, in one aspect, provided are kits including a first container comprising a mitochondrial lipid shunt modulator, e.g., as described herein, or a pharmaceutically acceptable salt, solvate, hydrate, tautomer, or stereoisomer thereof, or a pharmaceutical composition thereof. In certain embodiments, the kit of the disclosure includes a first container comprising a mitochondrial lipid shunt modulator, e.g., as described herein, or a pharmaceutically acceptable salt thereof, or a pharmaceutical composition thereof. In certain embodiments, the kits are useful in preventing and/or treating a disease, disorder, or condition described herein in a subject (e.g., a neurodegenerative disease, such as ALS). In certain embodiments, the kits further include instructions for administering the mitochondrial lipid shunt modulator, e.g., as described herein, or a pharmaceutically acceptable salt, solvate, hydrate, tautomer, or stereoisomer thereof, or a pharmaceutical composition thereof, to a subject to prevent and/or treat a neurodegenerative disease, e.g., ALS.

### ENUMERATED EMBODIMENTS

1. A method of modulating the oxidation of a fatty acid in a cell or subject exhibiting aberrant fatty acid oxidation, comprising administering to the cell or subject a mitochondrial lipid shunt modulator.
2. The method of embodiment 1, wherein the cell or subject exhibits aberrant fatty acid oxidation, e.g., prior to administration of the mitochondrial lipid shunt modulator.
3. The method of any one of embodiments 1-2, wherein the aberrant fatty acid oxidation comprises the prolongation of the elevation of fatty acid oxidation in response to starvation or metabolic stress.
4. The method of embodiment 3, wherein the aberrant fatty acid oxidation comprises the prolongation of the elevation of fatty acid oxidation in response to starvation.
5. The method of any one of embodiments 3-4, wherein the aberrant fatty acid oxidation comprises the prolongation of the elevation of fatty acid oxidation in response to metabolic stress.
6. The method of any one of embodiments 1-5, wherein the aberrant fatty acid oxidation comprises an increase (e.g., at least 1, 2, 3, 4, 5, 7.5, 10, 15, 20% increase) in fatty acid oxidation, e.g., compared with a reference standard (e.g., a healthy cell).
7. The method of embodiment 6, wherein the increase comprises an increase in fatty acid oxidation for about 5, 10, 15, 30, 45 minutes, 1, 1.5, 2, 3, 4, 5, 6, 8, 10, 12, 16, 20, 24, 30, 36, or 48 hours, e.g., compared with a reference standard (e.g., a healthy cell).
8. The method of any one of embodiments 1-7, wherein the aberrant fatty acid oxidation comprises an increase in cellular ATP levels (e.g., at least 1, 2, 3, 4, 5, 7.5, 10, 15, 20% increase), e.g., compared with a reference standard (e.g., a healthy cell).
9. The method of any one of embodiments 2-8, wherein the aberrant fatty acid oxidation results in the accumulation of oxidative damage in the cell or subject.
10. The method of any one of embodiments 2-8, wherein the aberrant fatty acid oxidation results in the accumulation of reactive oxygen species, acyl CoA, or carbonylation of proteins.
11. The method of any one of embodiments 1-11, wherein the modulating comprises reducing the oxidation of a fatty acid compared with a reference standard.
12. The method of embodiment 11, wherein the reducing comprises reducing the oxidation of a fatty acid by about 1, 2, 3, 4, 5, 7.5, 10, 15, 20%, e.g., compared to a reference standard.
13. The method of any one of embodiments 1-12, wherein the oxidation comprises beta-oxidation.
14. The method of any one of embodiments 1-13, wherein the oxidation is catalyzed by an enzyme.
15. The method of any one of embodiments 1-14, wherein the oxidation is catalyzed by an acyl CoA dehydrogenase.
16. The method of any one of embodiments 1-15, wherein the oxidation is catalyzed by hydroxyacyl-CoA dehydrogenase trifunctional multienzyme complex subunit alpha.
17. The method of any one of embodiments 1-16, wherein the fatty acid comprises a medium chain, long chain fatty acid or a very long chain fatty acid.
18. The method of any one of embodiments 1-17, wherein the fatty acid comprises a medium chain fatty acid.
19. The method of any one of embodiments 1-17, wherein the fatty acid comprises a long chain fatty acid.
20. The method of any one of embodiments 1-17, wherein the fatty acid comprises a very long chain fatty acid.
21. The method of any one of embodiments 1-17, wherein the fatty acid comprises linoleic acid, stearic acid, oleic acid, palmitic acid, linoelaidic acid, arachidonic acid, erucic acid, eicosapentaenoic acid, or docosahexaenoic acid.
22. The method of any one of embodiments 1-17, wherein the fatty acid comprises linoleic acid.
23. The method of any one of embodiments 1-17, wherein the fatty acid stearic acid.
24. The method of any one of embodiments 1-17, wherein the fatty acid oleic acid.
25. The method of any one of embodiments 1-17, wherein the fatty acid comprises palmitic acid.
26. The method of any one of embodiments 1-17, wherein the fatty acid comprises linoelaidic acid.
27. The method of any one of embodiments 1-17, wherein the fatty acid comprises arachidonic acid.
28. The method of any one of embodiments 1-17, wherein the fatty acid comprises 25 eicosapentaenoic acid.
29. The method of any one of embodiments 1-17, wherein the fatty acid comprises docosahexaenoic acid.
30. The method of any one of embodiments 1-29, wherein the cell or subject has a mutation in a gene.
31. The method of embodiment 30, wherein the gene is selected from a gene in the mitochondrial lipid shunt pathway.
32. The method of any one of embodiments 30-31, wherein the gene is selected from TDP43, FUS, OPTN, VCP, UBQLN1, CHCHD10, ALS2, ATX2, CCNF, C9orf72, SOD1, SETX, VAPB, SPG11, SQSTM1, PFN1, TBK1, and TIA1.
33. The method of any one of embodiments 30-31, wherein the gene is selected from TDP43.
34. The method of any one of embodiments 30-31, wherein the gene is FUS.
35. The method of any one of embodiments 30-31, wherein the gene is OPTN.
36. The method of any one of embodiments 30-31, wherein the gene is VCP.
37. The method of any one of embodiments 30-31, wherein the gene is UBQLN1.
38. The method of any one of embodiments 30-31, wherein the gene is CHCHD10.
39. The method of any one of embodiments 30-31, wherein the gene is ALS2.
40. The method of any one of embodiments 30-31, wherein the gene is ATX2.
41. The method of any one of embodiments 30-31, wherein the gene is CCNF.
42. The method of any one of embodiments 30-31, wherein the gene is C9orf72.
43. The method of any one of embodiments 30-31, wherein the gene is SOD1.
44. The method of any one of embodiments 30-31, wherein the gene is SETX.
45. The method of any one of embodiments 30-31, wherein the gene is VAPB.
46. The method of any one of embodiments 30-31, wherein the gene is SPG11.
47. The method of any one of embodiments 30-31, wherein the gene is SQSTM1.
48. The method of any one of embodiments 30-31, wherein the gene is PFN1.
49. The method of any one of embodiments 30-31, wherein the gene is TBK1.
50. The method of any one of embodiments 30-31, wherein the gene is TIA1.
51. The method of any one of embodiments 30-32 and 34, wherein the mutation is a mutation in the FUS gene.
52. The method of embodiment 51, wherein the FUS mutation is selected from R521G and R522G.
53. The method of embodiment 51 or 52, wherein the FUS mutation is R521G.
54. The method of embodiment 51 or 52, wherein the FUS mutation is R522G.
55. The method of any one of embodiments 30-33, wherein the mutation is a mutation in the TDP43 gene.
56. The method of embodiment 55, wherein the TDP43 mutation is selected from Q331K, M337V, and G298S.
57. The method of embodiment 55, wherein the TDP43 mutation is Q331K.
58. The method of embodiment 55, wherein the TDP43 mutation is M337V.
59. The method of embodiment 55, wherein the TDP43 mutation is G298S.
60. The method of any one of embodiments 30-32 and 43, wherein the mutation is a mutation in the SOD1 gene.
61. The method of embodiment 60, wherein the SOD1 mutation is selected from G93A, G37R, and G85R.
62. The method of embodiment 60, wherein the SOD1 mutation is G93A.
63. The method of embodiment 60, wherein the SOD1 mutation is G37R.
64. The method of embodiment 60, wherein the SOD1 mutation is G85R.
65. The method of any one of embodiments 1-64, wherein the cell or subject further comprises a decrease in fatty acid import into lipid droplets, e.g., compared with a reference standard.
66. The method of embodiment 65, wherein the cell or subject further comprises a decrease in lipid droplet size and/or lipid droplet number (e.g., about 1.5×, 2×, 3×, 4×, 5×, or 10×), e.g., compared with a reference standard.
67. The method of any one of embodiments 1-66, wherein the mitochondrial lipid shunt modulator comprises a VDAC modulator, a CPT1 modulator, or an ACSL modulator.
68. The method of any one of embodiments 1-67, wherein the mitochondrial lipid shunt modulator comprises a VDAC modulator.
69. The method of any one of embodiments 1-67, wherein the mitochondrial lipid shunt modulator comprises a CPT1 modulator.
70. The method of any one of embodiments 1-67, wherein the mitochondrial lipid shunt modulator comprises an ACSL modulator.
71. The method of any one of embodiments 1-70, wherein the mitochondrial lipid shunt modulator comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) e.g., as described herein.
72. The method of any one of embodiments 1-70, wherein the mitochondrial lipid shunt modulator comprises a compound of Formula (I), e.g., as described herein.
73. The method of any one of embodiments 1-70, wherein the mitochondrial lipid shunt modulator comprises a compound of Formula (II), e.g., as described herein.
74. The method of any one of embodiments 1-70, wherein the mitochondrial lipid shunt modulator comprises a compound of Formula (III), e.g., as described herein.
75. The method of any one of embodiments 1-70, wherein the mitochondrial lipid shunt modulator comprises a compound of Formula (IV), e.g., as described herein.
76. The method of any one of embodiments 1-70, wherein the mitochondrial lipid shunt 25 modulator comprises a compound of Formula (V), e.g., as described herein.
77. The method of any one of embodiments 1-70, wherein the mitochondrial lipid shunt modulator comprises a compound of Formula (VI), e.g., as described herein.
78. The method of any one of embodiments 1-70, wherein the mitochondrial lipid shunt modulator comprises a compound of Formula (VII), e.g., as described herein.
79. The method of any one of embodiments 1-70, wherein the mitochondrial lipid shunt modulator comprises a compound of Formula (VIII), e.g., as described herein.
80. The method of any one of embodiments 1-70, wherein the mitochondrial lipid shunt modulator comprises a compound of Formula (IX), e.g., as described herein.
81. The method of any one of embodiments 1-70, wherein the mitochondrial lipid shunt modulator comprises a compound of Formula (X), e.g., as described herein.
82. The method of any one of embodiments 1-70, wherein the mitochondrial lipid shunt modulator comprises a compound of Formula (XI), e.g., as described herein.
83. The method of any one of embodiments 1-70, wherein the mitochondrial lipid shunt modulator comprises a compound of Formula (XII), e.g., as described herein.
84. The method of any one of embodiments 1-70, wherein the mitochondrial lipid shunt modulator comprises a compound of Formula (XIII), e.g., as described herein.
85. The method of any one of embodiments 1-70, wherein the mitochondrial lipid shunt modulator comprises a compound of Formula (XIV), e.g., as described herein.
86. The method of any one of embodiments 1-70, wherein the mitochondrial lipid shunt modulator comprises a compound of Formula (XV), e.g., as described herein.
87. The method of any one of embodiments 1-70, wherein the mitochondrial lipid shunt modulator comprises a compound of Formula (XVI), e.g., as described herein.
88. The method of any one of embodiments 1-70, wherein the mitochondrial lipid shunt 30 modulator comprises a compound of Formula (XVII), e.g., as described herein.
89. The method of any one of embodiments 1-70, wherein the mitochondrial lipid shunt modulator comprises a compound of Formula (XVIII), e.g., as described herein.
90. The method of any one of embodiments 1-70, wherein the mitochondrial lipid shunt modulator comprises a compound of Formula (XIX), e.g., as described herein.
91. The method of any one of embodiments 1-90, wherein the mitochondrial lipid shunt modulator comprises a compound selected from (1)-(573) or a pharmaceutically acceptable salt or ester thereof, e.g., as described herein.
92. The method of any one of embodiments 1-91, wherein the mitochondrial lipid shunt modulator is selected from the group consisting of: erastin, erstin, efsevin, olesoxime, teglicar, perhexiline, amiodarone, 2-tetradecylglydate, PA22, AG-17, AG-825, AS-604850, BAY 43-9006, CAY 10561, CAY 10571, D4476, sulindac, sulindac sulfone (exisulind), itraconazole, torin-1, nintedanib, ruxolitinib, SB-505124, SB-203580, leelamine, LY294002, LY364947, ML-9, O-1918, PD169316, emodin, KN-93, bisindolylmaleimide IX, 17β-hydroxywortmannin, GSK1059615, (S)-H-1152, PD 166326, PI3-Kinase alpha inhibitor 2, piceatannol, pimasertib, RG-13022, roscovitine, SB-202190, SB-415286, SMI-4a, sphingosine inhibitor compound, TGX-221, U0126, and WEHI-9625, or an analog thereof.
93. The method of any one of embodiments 1-92, wherein the mitochondrial lipid shunt modulator is selected from the group consisting of: sulindac and sulindac sulfone (exisulind).
94. The method of any one of embodiments 1-93, wherein the mitochondrial lipid shunt modulator is sulindac.
95. The method of any one of embodiments 1-93, wherein the mitochondrial lipid shunt modulator is sulindac sulfone.
96. The method of any one of embodiments 1-92, wherein the mitochondrial lipid shunt modulator is (S)-H-1152.
97. The method of any one of embodiments 1-92, wherein the mitochondrial lipid shunt modulator is ruxolitinib.
98. The method of any one of embodiments 1-92, wherein the mitochondrial lipid shunt modulator is itraconazole.
99. The method of any one of embodiments 1-92, wherein the mitochondrial lipid shunt modulator is erastin.
100. The method of any one of embodiments 1-92, wherein the mitochondrial lipid shunt modulator is erstin.
101. The method of any one of embodiments 1-92, wherein the mitochondrial lipid shunt modulator is efsevin.
102. The method of any one of embodiments 1-92, wherein the mitochondrial lipid shunt modulator is olesoxime.
103. The method of any one of embodiments 1-92, wherein the mitochondrial lipid shunt modulator is teglicar.
104. The method of any one of embodiments 1-92, wherein the mitochondrial lipid shunt modulator is perhexiline.
105. The method of any one of embodiments 1-92, wherein the mitochondrial lipid shunt modulator is amiodarone.
106. The method of any one of embodiments 1-92, wherein the mitochondrial lipid shunt 30 modulator is 2-tetradecylglydate.
107. The method of any one of embodiments 1-92, wherein the mitochondrial lipid shunt modulator is PA22.
108. The method of any one of embodiments 1-92, wherein the mitochondrial lipid shunt modulator is CAY10561.
109. The method of any one of embodiments 1-92, wherein the mitochondrial lipid shunt modulator is WEHI-9625.
110. The method of any one of embodiments 1-92, wherein the mitochondrial lipid shunt modulator is leelamine.
111. The method of any one of embodiments 1-92, wherein the mitochondrial lipid shunt modulator is 17p-hydroxywortmannin.
112. The method of any one of embodiments 1-92, wherein the mitochondrial lipid shunt modulator is KN-93.
113. The method of any one of embodiments 1-92, wherein the mitochondrial lipid shunt 20 modulator is SMI-4a.
114. The method of any one of embodiments 1-92, wherein the mitochondrial lipid shunt modulator is nintedanib.
115. The method of any one of embodiments 1-92, wherein the mitochondrial lipid shunt modulator is LY364947.
116. The method of any one of embodiments 1-92, wherein the mitochondrial lipid shunt modulator is emodin.
117. The method of any one of embodiments 1-92, wherein the mitochondrial lipid shunt modulator is D4476.
117. The method of any one of embodiments 1-92, wherein the mitochondrial lipid shunt modulator is SB-505124.
118. The method of any one of embodiments 1-92, wherein the mitochondrial lipid shunt modulator is torin-1.
119. The method of any one of embodiments 1-92, wherein the mitochondrial lipid shunt modulator is SB-203580.
120. The method of any one of embodiments 1-92, wherein the mitochondrial lipid shunt modulator is LY294002.
121. The method of any one of embodiments 1-92, wherein the mitochondrial lipid shunt modulator is ML-9.
122. The method of any one of embodiments 1-92, wherein the mitochondrial lipid shunt 20 modulator is roscovitine.
123. The method of any one of embodiments 1-92, wherein the mitochondrial lipid shunt modulator is TGX-221.
124. The method of any one of embodiments 1-92, wherein the mitochondrial lipid shunt modulator is pimasertib.
125. The method of any one of embodiments 1-92, wherein the mitochondrial lipid shunt modulator is piceatannol.
126. The method of any one of embodiments 1-92, wherein the mitochondrial lipid shunt modulator is RG-13022.
127. The method of any one of embodiments 1-92, wherein the mitochondrial lipid shunt modulator is bisindolylmaleimide IX.
128. The method of any one of embodiments 1-92, wherein the mitochondrial lipid shunt modulator is O-1918.
129. The method of any one of embodiments 1-92, wherein the mitochondrial lipid shunt modulator is SB-202190.
130. The method of any one of embodiments 1-92, wherein the mitochondrial lipid shunt modulator is SB-415286.
131. The method of any one of embodiments 1-130, wherein the mitochondrial lipid shunt modulator modulates the binding of VDAC2 to GSK3 or hexokinase.
132. The method of any one of embodiments 1-131, wherein the mitochondrial lipid shunt modulator modulates the binding of VDAC2 to GSK3.
133. The method of any one of embodiments 1-131, wherein the mitochondrial lipid shunt modulator modulates the binding of VDAC2 to hexokinase.
134. The method of any one of embodiments 1-132, wherein the modulating of GSK binding to VDAC2 results in modulating VDAC2 activity.
135. The method of any one of embodiments 1-132 or 134, wherein the modulating of GSK binding to VDAC2 results in modulating the phosphorylation state of VDAC2.
136. The method of any one of embodiments 1-135, wherein the mitochondrial lipid shunt modulator modulates the expression of VDAC or VDAC2.
137. The method of any one of embodiments 1-136, wherein the mitochondrial lipid shunt modulator modulates the expression of VDAC.
138. The method of any one of embodiments 1-136, wherein the mitochondrial lipid shunt modulator modulates the expression of VDAC 2.
139. The method of embodiments 1-138, wherein the mitochondrial lipid shunt modulator is an inhibitor of a protein kinase that regulates the activity of GSK3 binding to VDAC2, directly or through a regulatory pathway.
140. The method of embodiments 1-54, wherein the modulating of GSK3 binding to VDAC2 is achieved by modulating the activity of upstream kinases, e.g., kinases that deactivate or modulate GSK3 activity.
141. The method of any one of embodiment 140, wherein the mitochondrial lipid shunt modulator binds to carnitine palmitoyltransferase I (CPTI).
142. The method of any one of embodiments 1-141, wherein the mitochondrial lipid shunt modulator binds to long chain acyl CoA synthetase (ACSL).
143. The method of any one of embodiments 1-142, wherein the modulating of fatty acid oxidation comprises modulating the structural organization VDAC2 clustering in the mitochondrial membrane surface.
143. A method of modulating the formation of stress granules in a cell or subject exhibiting aberrant fatty acid oxidation, comprising administering to the cell or subject a mitochondrial lipid shunt modulator.
144. The method of embodiment 143, wherein the modulating comprises increasing the formation of stress granules (e.g., assisting formation of stress granules).
145. The method of any one of embodiments 143 or 144, wherein increasing stress granule formation results in modulating VDAC2 activity.
145. The method of any one of embodiments 142-144, wherein increasing stress granule formation results in decreasing VDAC2 activity.
147. The method of embodiment 145 or 146, wherein increasing stress granule formation results in reduction of aberrant fatty acid oxidation.
148. The method of any one of embodiments 145-147, wherein the modulating comprises modulating the association of stress granules with the mitochondria and/or lipid droplets.
149. A method of modulating one or more of:
   (i) the number of stress granules;
   (ii) morphology of a stress granule, e.g., the size, shape, surface area to volume ratio;
   (iii) the protein or nucleic acid composition of a stress granule;
   (iv) the internal mobility or internal dynamics of a stress granule; and
   (v) the phase of a stress granule;
   in a cell or subject exhibiting aberrant fatty acid oxidation, comprising administering to the cell or subject a mitochondrial lipid shunt modulator.
150. The method of embodiment 149, comprising (i).
151. The method of any one of embodiments 149-150, comprising (ii).
152. The method of any one of embodiments 149-151, comprising (iii).
153. The method of any one of embodiments 149-152, comprising (iv).
154. The method of any one of embodiments 149-153 comprising (v).
155. A method of modulating the formation of lipid droplets in a cell or subject exhibiting aberrant fatty acid oxidation, comprising administering to the cell or subject a mitochondrial lipid shunt modulator.
156. The method of embodiment 155, wherein the modulating comprises reducing the formation of lipid droplets and/or preventing association of lipid droplets with the mitochondria or stress granules.
157. A method of modulating one or more of:
   (i) the number of lipid droplets;
   (ii) morphology of a lipid droplet, e.g., the size, shape, surface area to volume ratio;
   (iii) the protein or nucleic acid composition of a lipid droplet;
   (iv) the internal mobility or internal dynamics of a lipid droplet;
   (v) the phase of a lipid droplet; and
   (vi) the agglomeration state of a lipid droplet;
   in a cell or subject exhibiting aberrant fatty acid oxidation, comprising administering to the cell or subject a mitochondrial lipid shunt modulator.
158. The method of embodiment 157, comprising (i).
159. The method of any one of embodiments 157-158, comprising (ii).
160. The method of any one of embodiment 157-159, comprising (iii).
161. The method of any one of embodiments 157-160 comprising (iv).
162. The method of any one of embodiments 157-161, comprising (v).
163. The method of any one of embodiments 157-162, comprising (vi).
164. The method of any one of embodiments 149-163, wherein modulating the formation and/or size or number of lipid droplets results in modulating VDAC2.
165. The method of any one of embodiments 149-164, wherein the mitochondrial lipid shunt modulator comprises a VDAC modulator, a CPT1 modulator, or an ACSL modulator.
166. The method of any one of embodiments 149-165, wherein the mitochondrial lipid shunt modulator comprises a VDAC modulator.
167. The method of any one of embodiments 149-166, wherein the mitochondrial lipid shunt modulator comprises a CPT1 modulator.
168. The method of any one of embodiments 149-167, wherein the mitochondrial lipid shunt modulator comprises an ACSL modulator.
169. The method of any one of 149-168, wherein the mitochondrial lipid shunt modulator comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt thereof, e.g., as described herein.
170. The method of any one of embodiments 149-169, wherein the mitochondrial lipid shunt modulator comprises a compound of Formula (I) or a pharmaceutically acceptable salt thereof, e.g., as described herein.
171. The method of any one of embodiments 149-169, wherein the mitochondrial lipid shunt modulator comprises a compound of Formula (II) or a pharmaceutically acceptable salt thereof, e.g., as described herein.
172. The method of any one of embodiments 149-169, wherein the mitochondrial lipid shunt modulator comprises a compound of Formula (III) or a pharmaceutically acceptable salt thereof, e.g., as described herein.
173. The method of any one of embodiments 149-169, wherein the mitochondrial lipid shunt modulator comprises a compound of Formula (IV) or a pharmaceutically acceptable salt thereof, e.g., as described herein.
174. The method of any one of embodiments 149-169, wherein the mitochondrial lipid shunt modulator comprises a compound of Formula (V) or a pharmaceutically acceptable salt thereof, e.g., as described herein.
175. The method of any one of embodiments 149-169, wherein the mitochondrial lipid shunt modulator comprises a compound of Formula (VI) or a pharmaceutically acceptable salt thereof, e.g., as described herein.
176. The method of any one of embodiments 149-169, wherein the mitochondrial lipid shunt modulator comprises a compound of Formula (VII) or a pharmaceutically acceptable salt thereof, e.g., as described herein.
177. The method of any one of embodiments 149-169, wherein the mitochondrial lipid shunt modulator comprises a compound of Formula (VIII) or a pharmaceutically acceptable salt thereof, e.g., as described herein.
178. The method of any one of embodiments 149-169, wherein the mitochondrial lipid shunt modulator comprises a compound of Formula (IX) or a pharmaceutically acceptable salt thereof, e.g., as described herein.
179. The method of any one of embodiments 149-169, wherein the mitochondrial lipid shunt modulator comprises a compound of Formula (X) or a pharmaceutically acceptable salt thereof, e.g., as described herein.
180. The method of any one of embodiments 149-169, wherein the mitochondrial lipid shunt modulator comprises a compound of Formula (XI) or a pharmaceutically acceptable salt thereof, e.g., as described herein.
181. The method of any one of embodiments 149-169, wherein the mitochondrial lipid shunt modulator comprises a compound of Formula (XII) or a pharmaceutically acceptable salt thereof, e.g., as described herein.
182. The method of any one of embodiments 149-169, wherein the mitochondrial lipid shunt modulator comprises a compound of Formula (XIII) or a pharmaceutically acceptable salt thereof, e.g., as described herein.
183. The method of any one of embodiments 149-169, wherein the mitochondrial lipid shunt modulator comprises a compound of Formula (XIV) or a pharmaceutically acceptable salt thereof, e.g., as described herein.
185. The method of any one of embodiments 149-169, wherein the mitochondrial lipid shunt modulator comprises a compound of Formula (XV) or a pharmaceutically acceptable salt thereof, e.g., as described herein.
186. The method of any one of embodiments 149-1699, wherein the mitochondrial lipid shunt modulator comprises a compound of Formula (XVI) or a pharmaceutically acceptable salt thereof, e.g., as described herein.
187. The method of any one of embodiments 149-169, wherein the mitochondrial lipid shunt modulator comprises a compound of Formula (XVII) or a pharmaceutically acceptable salt thereof, e.g., as described herein.
188. The method of any one of embodiments 149-169, wherein the mitochondrial lipid shunt modulator comprises a compound of Formula (XVIII) or a pharmaceutically acceptable salt thereof, e.g., as described herein.
189. The method of any one of embodiments 149-188, wherein the mitochondrial lipid shunt modulator comprises a compound selected from (1)-(573) or a pharmaceutically acceptable salt or ester thereof, e.g., as described herein.
190. The method of any one of embodiments 149-189, wherein the mitochondrial lipid shunt modulator is selected from erastin, erstin, efsevin, olesoxime, teglicar, 2-tetradecylglydate, PA22, AG-17, AG-825, AS-604850, BAY 43-9006, CAY 10561, CAY 10571, D4476, sulindac, sulindac sulfone (exisulind), itraconazole, torin-1, nintedanib, ruxolitinib, SB-505124, SB-203580, leelamine, LY294002, LY364947, ML-9, O-1918, PD169316, emodin, KN-93, bisindolylmaleimide IX, 170-hydroxywortmannin. GSK1059615, (S)-H-1152, PD 166326, PI3-Kinase alpha inhibitor 2, piceatannol, pimasertib, RG-13022, roscovitine, SB-202190, SB-415286, SMI-4a, sphingosine inhibitor compound, TGX-221, U0126, and WEHI-9625, or an analog thereof.
191. The method of any one of embodiments 149-190, wherein the mitochondrial lipid shunt modulator is selected from the group consisting of: sulindac, sulindac sulfone (exisulind).
192. The method of any one of embodiments 149-190, wherein the mitochondrial lipid shunt modulator is sulindac.
193. The method of any one of embodiments 149-190, wherein the mitochondrial lipid shunt modulator is sulindac sulfone.
194. The method of any one of embodiments 149-190, wherein the mitochondrial lipid shunt modulator is (S)-H-1152.
195. The method of any one of embodiments 148-189, wherein the mitochondrial lipid shunt modulator is ruxolitinib.
196. The method of any one of embodiments 149-190, wherein the mitochondrial lipid shunt modulator is itraconazole.
197. The method of any one of embodiments 149-190, wherein the mitochondrial lipid shunt modulator is erastin.
198. The method of any one of embodiments 149-190, wherein the mitochondrial lipid shunt modulator is erstin.
199. The method of any one of embodiments 149-190, wherein the mitochondrial lipid shunt modulator is efsevin.
200. The method of any one of embodiments 149-190, wherein the mitochondrial lipid shunt modulator is olesoxime.
201. The method of any one of embodiments 149-190, wherein the mitochondrial lipid shunt modulator is teglicar.
202. The method of any one of embodiments 149-190, wherein the mitochondrial lipid shunt modulator is perhexiline.
203. The method of any one of embodiments 149-190, wherein the mitochondrial lipid shunt modulator is amiodarone.
204. The method of any one of embodiments 149-190, wherein the mitochondrial lipid shunt modulator is 2-tetradecylglydate.
205. The method of any one of embodiments 149-190, wherein the mitochondrial lipid shunt modulator is PA22.
206. The method of any one of embodiments 149-190, wherein the mitochondrial lipid shunt modulator is CAY10561.
207. The method of any one of embodiments 149-190, wherein the mitochondrial lipid shunt modulator is WEHI-9625.
208. The method of any one of embodiments 149-190, wherein the mitochondrial lipid shunt modulator is leelamine.
209. The method of any one of embodiments 149-190, wherein the mitochondrial lipid shunt modulator is 17p-hydroxywortmannin.
210. The method of any one of embodiments 149-190, wherein the mitochondrial lipid shunt modulator is KN-93.
211. The method of any one of embodiments 149-190, wherein the mitochondrial lipid shunt modulator is SMI-4a.
212. The method of any one of embodiments 149-190, wherein the mitochondrial lipid shunt modulator is nintedanib.
213. The method of any one of embodiments 149-190, wherein the mitochondrial lipid shunt modulator is LY364947.
214. The method of any one of embodiments 149-190, wherein the mitochondrial lipid shunt modulator is emodin.
215. The method of any one of embodiments 149-190, wherein the mitochondrial lipid shunt modulator is D4476.
216. The method of any one of embodiments 149-190, wherein the mitochondrial lipid shunt modulator is SB-505124.
217. The method of any one of embodiments 149-190, wherein the mitochondrial lipid shunt modulator is torin-1.
218. The method of any one of embodiments 149-190, wherein the mitochondrial lipid shunt modulator is SB-203580.
219. The method of any one of embodiments 149-190, wherein the mitochondrial lipid shunt modulator is LY294002.
220. The method of any one of embodiments 149-190, wherein the mitochondrial lipid shunt modulator is ML-9.
221. The method of any one of embodiments 149-190, wherein the mitochondrial lipid shunt modulator is roscovitine.
222. The method of any one of embodiments 149-190, wherein the mitochondrial lipid shunt modulator is TGX-221.
223. The method of any one of embodiments 149-190, wherein the mitochondrial lipid shunt modulator is pimasertib.
224. The method of any one of embodiments 149-190, wherein the mitochondrial lipid shunt modulator is piceatannol.
225. The method of any one of embodiments 149-190, wherein the mitochondrial lipid shunt modulator is RG-13022.
226. The method of any one of embodiments 149-190, wherein the mitochondrial lipid shunt modulator is bisindolylmaleimide IX.
227. The method of any one of embodiments 149-190, wherein the mitochondrial lipid shunt modulator is O-1918.
228. The method of any one of embodiments 149-190, wherein the mitochondrial lipid shunt modulator is SB-202190.
229. The method of any one of embodiments 149-190, wherein the mitochondrial lipid shunt modulator is SB-415286.
230. The method of any one of embodiments 149-229, wherein the mitochondrial lipid shunt modulator modulates the binding of VDAC2 to GSK3 or hexokinase.
231. The method of any one of embodiments 149-230, wherein the mitochondrial lipid shunt modulator modulates the binding of VDAC2 to GSK3.
232. The method of any one of embodiments 149-230, wherein the mitochondrial lipid shunt modulator modulates the binding of VDAC2 to hexokinase.
233. The method of any one of embodiments 149-232, wherein the modulating of GSK binding to VDAC2 results in modulating VDAC2 activity.
234. The method of any one of embodiments 149-231 and 233, wherein the modulating of GSK binding to VDAC2 results in modulating the phosphorylation state of VDAC2.
235. The method of any one of embodiments 149-234, wherein the mitochondrial lipid shunt modulator modulates the expression of VDAC or VDAC2.
236. The method of any one of embodiments 149-235, wherein the mitochondrial lipid shunt modulator modulates the expression of VDAC.
237. The method of any one of embodiments 147-235, wherein the mitochondrial lipid shunt modulator modulates the expression of VDAC2.
238. The method of embodiments 149-237, wherein the mitochondrial lipid shunt modulator is an inhibitor of a protein kinase that regulates the activity of GSK3 binding to VDAC2, directly or through a regulatory pathway.
239. The method of embodiments 149-238, wherein the modulating of GSK3 binding to VDAC2 is achieved by modulating the activity of upstream kinases, e.g., kinases that deactivate or modulate GSK3 activity.
240. The method of any one of embodiments 149-239, wherein the mitochondrial lipid shunt modulator binds to carnitine palmitoyltransferase I (CPTI).
241. The method of any one of embodiments 149-240, wherein the mitochondrial lipid shunt modulator binds to long chain acyl CoA synthetase (ACSL).
242. The method of any one of embodiments 149-241, wherein the modulating of fatty acid oxidation comprises modulating the structural organization VDAC2 clustering in the mitochondrial membrane surface.
243. A method of treating a neurodegenerative disease, e.g., amyotrophic lateral sclerosis, in a subject, wherein the subject (e.g., a cell in the subject) exhibits one or more of the following:
   i) aberrant fatty acid oxidation;
   ii) reduced fatty acid import into lipid droplets;
   iii) reduced size and/or number of lipid droplets;
   iv) decreased formation of stress granules;
   v) formation of aberrant stress granules; and
   vi) impaired VDAC2 function;
   the method comprising administering to the subject a mitochondrial lipid shunt modulator.
244. The method of embodiment 243, comprising (i).
245. The method of any one of embodiments 243-244, comprising (iI).
246. The method of any one of embodiments 243-245, comprising (iii).
247. The method of any one of embodiments 243-246, comprising (iv).
248. The method of any one of embodiments 243-247, comprising (v).
249. The method of any one of embodiments 243-248, comprising (vi).
250. A method of treating a neurodegenerative disease, e.g., amyotrophic lateral sclerosis, in a subject, wherein the subject has or is identified as having a mutation in one or more of the following genes:
   i) TDP-43;
   ii) C9orf72; and
   iii) FUS;
   the method comprising administering to the subject a mitochondrial lipid shunt modulator.
251. The method of embodiment 250, wherein the subject has a mutation in (i).
252. The method of embodiment 251, wherein the subject has a mutation in (ii).
253. The method of embodiment 252, wherein the subject has a mutation in (iii).
254. A method of evaluating a compound for the ability to act as a mitochondrial lipid shunt modulator, modulate fatty acid oxidation, regulate lipid droplet size or number, and/or regulate stress granule formation, comprising:
   i) providing a library comprising a plurality of compounds;
   ii) providing a plurality of cells, wherein one or more cells of the plurality is disposed in a reaction chamber;
   iii) contacting a cell in the reaction chamber with a compound of the plurality of compounds; and
   iv) evaluating a cell for modulation of fatty acid oxidation, regulation of lipid droplet size or number, agglomeration state, and/or regulation of stress granule formation.
255. The method of embodiment 254, wherein plurality of compounds comprises a VDAC modulator, a CPT1 modulator, or an ACSL modulator.
256. The method of embodiment 254, wherein plurality of compounds comprises a VDAC modulator.
257. The method of embodiment 254, wherein plurality of compounds comprises a CPT1 modulator.
258. The method of embodiment 254, wherein plurality of compounds comprises a ACSL modulator.
259. The method of any one of embodiments 254-258, wherein the plurality of compounds comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt thereof, e.g., as described herein.
260. The method of any one of embodiments 254-259, wherein the mitochondrial lipid shunt modulator is selected from erastin, erstin, efsevin, olesoxime, teglicar, 2-tetradecylglydate, PA22, AG-17, AG-825, AS-604850, BAY 43-9006, CAY 10561, CAY 10571, D4476, sulindac, sulindac sulfone (exisulind), itraconazole, torin-1, nintedanib, ruxolitinib, SB-505124, SB-203580, leelamine, LY294002, LY364947, ML-9, O-1918, PD169316, emodin, KN-93, bisindolylmaleimide IX, 17p-hydroxywortmannin, GSK1059615, (S)-H-1152, PD 166326, PI3-Kinase alpha inhibitor 2, piceatannol, pimasertib, RG-13022, roscovitine, SB-202190, SB-415286, SMI-4a, sphingosine inhibitor compound, TGX-221, U0126, and WEHI-9625, or an analog thereof.
261. The method of any one of embodiments 254-260, wherein the one or more cell comprises a TDP43, FUS, OPTN, VCP, UBQLN1, CHCHD10, ALS2, ATX2, CCNF, C9orf72, SOD1, SETX, VAPB, SPG11, SQSTM1, PFN1, TBK1, or TIA1, mutation
262. The method of any one of embodiments 254-261, wherein the evaluating comprises evaluating a cell for modulation of fatty acid oxidation.
263. The method of any one of embodiments 254-262, wherein the evaluating comprises regulation of lipid droplet size or number.
264. A method of evaluating the effect of a compound or plurality of compounds administered to a cell or human subject, for the ability to act as a mitochondrial lipid shunt modulator, modulate fatty acid oxidation, regulate lipid droplet size or number, and/or regulate stress granule formation, and the effects of which will be correlated with established a neurodegenerative disease (e.g., amyotrophic lateral sclerosis) or a pathophysiological disease marker, for use as:
   i) a disease status diagnostic
   ii) a disease progression diagnostic/biomarker
   iii) a biomarker or diagnostic of therapeutic efficacy of administered compounds.
265. The method of any one of embodiments 1-264, wherein the mitochondrial lipid shunt modulator results in an increase in stress granule number and/or size, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard.
266. The method of any one of embodiments 1-264, wherein the increase in stress granule number and/or size is greater than 25%, 50%, 75%, or more.
267. The method of any one of embodiments 1-266, wherein the mitochondrial lipid shunt modulator results in an increase in lipid droplet number and/or size, e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or more, e.g., compared to a reference standard.
268. The method of any one of embodiments 1-267, wherein the increase in lipid droplet number and/or size is greater than 25%, 50%, 75%, or more.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1: Evaluating Lipid Shunt Modulators for Effect on Lipid Droplet Size

Knockout and endogenously tagged HEK293T cells were constructed using CRISPR/Cas9 protocol and plasmids described in Ran et. al (Ran et al., 2013). Knockout cell 25 lines were verified by western blotting, immunofluorescence. Genomic DNA was sequenced to verify disrupted region in knockout or fidelity of endogenous tagging. Endogenous tagging was performed by fusing tagging construct pCtag (linker-GFP/DDR2/mCherry/mCherry-VenusC-polyA-Puromycin) to the region upstream of the stop codon (˜500 bp) and downstream of stop codon (˜500 bp). PCR product containing homologous regions flanking the tagging construct was 30 co-transfected with px330-gRNA corresponding construct. Endogenous tagging was verified by western blotting, immunofluorescence staining, and genomic DNA sequencing.

Endogenous tagging of TDP43 was performed by fusing tagging construct pCtag-GFP-PURO (Amen and Kaganovich, STAR Protocols (2020)) to the region upstream of the stop codon (˜500 bp) and downstream of stop codon (˜500 bp). Q331K mutation was introduced by site directed mutagenesis. PCR product containing homologous regions (with the Q331K mutation) flanking the tagging construct was co-transfected with px330-gRNA corresponding construct (Ran et al., 2013). Endogenous tagging was verified by western blotting, immunofluorescence staining, and genomic DNA sequencing. CRISPR specificity was profiled using Digenome-Seq web tool (http://www.rgenome.net/cas-offinder/) (Bae et al., 2014). The following target sequences are used to modify genomic DNA: endogenous tagging of TDP43-‘GTCTTCTGGCTGGGGAATGTAGACAG’

Other ALS-associated mutants can be introduced similarly with different px330-gRNA plasmid and dsDNA fragments (80-100 bp, containing the mutation) corresponding to the mutation region.

To prepare the cells in microtiter plates, HEK293T cells were maintained in high glucose DMEM supplemented with 10% fetal bovine serum (FBS), 1% penicillin/streptomycin, at 37° C./5% CO2, SH-SY5Y cells were maintained in high glucose 1:1 F12/DMEM media supplemented with 10% FBS, 1% penicillin/streptomycin at 37° C./5% CO2. Cells modified via CRISPR/Cas9 were maintained as above with addition of puromycin (2 pg/ml, Sigma) during selection of clonal populations. Cells were seeded into microtiter plates at 50% confluency.

Exemplary lipid shunt modulators were dissolved in DMSO and introduced to the cells seeded in plates. After the cells were allowed to equilibrate with the compounds, the cells were subjected to a chosen stressor, such as starvation. The stress could be applied either before or after introduction of the compounds.

### Example 2: Evaluation of Exemplary Compounds on Stress Granule Size and Morphology

Stress Granules (SGs) were visualized following 12 hrs of starvation (media lacking glucose, lipids, and amino acids), and size/morphology analyzed using image analysis software, eg, NIS elements. We constructed a CRISPR/Cas9 tagged polyA-binding protein (PABPC1) fused to a photoconvertible fluorescent protein Dendra2 in HEK293T cell line to assess Stress Granule formation kinetics and internal dynamics (Gurskaya et al., 2006, Ran et al., 2013, Chudakov et al., 2007a). PABPC1 is an abundant Stress Granule component (Aulas et al., 2017, Jain et al., 2016). Stress Granule internal dynamics were measured by photoconverting the Dendra2 fluorophore from green to red in one point of the Stress Granule, and measuring the time needed for equilibration of the red color. Stress Granules are judged to have aberrant internal dynamics if the fluorophore equilibration takes longer than healthy controls in a statistically significant manner.

### Example 3: Evaluation of Exemplary Compounds on Lipid Droplet Number, Morphology, and Size

Lipid Droplets (LDs) are visualized with both BODIPY™ 558/568 C12 (4,4-Difluoro-5-(2-Thienyl)-4-Bora-3a,4α-Diaza-s-Indacene-3-Dodecanoic Acid, Thermo Fischer Scientific) and Bodipy (BODIPY™ 493/503 (4,4-Difluoro-1,3,5,7,8-Pentamethyl-4-Bora-3a,4α-Diaza-s-Indacene). Bodipy is a live cell dye, that selectively stains lipid droplets, and Bodipy-C12 is a fluorescent fatty acid (C12) that accumulates in the lipid droplets. This co-staining will allow us to identify to what extent fatty acids are accumulating in the lipid droplets, by using the [FI [Bodipy-C12]/[Bodipy]]ₑₓₚₑᵣᵢₘₑₙₜ/[FI [Bodipy-C12]/[Bodipy]]_{control} formula. For live cell imaging we will 384-well microscope glass bottom plates (IBIDI), or Cellview cell culture dish (Greiner Bio One). Confocal images are acquired using a fluorescent microscope, temperature and CO₂ incubator, using a 60× PlanApo VC oil objective NA 1.40. 488 nm, and 561 nm (Coherent, OBIS) lasers will be used to visualize the Bodipy and Bodipy-C12 staining. Number and size of lipid droplet clusters can be measured with standard image analysis software, including NIS Elements.

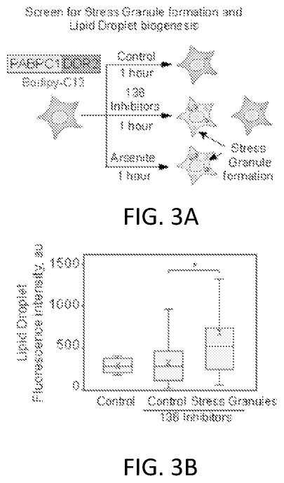

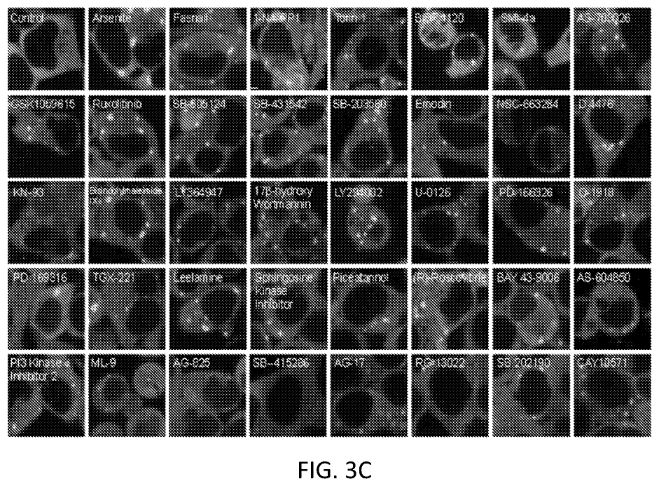

A library of 136 small molecule inhibitors (listed in Table 1) was screened for concurrent effects on fatty acid accumulation and SG formation. Cells expressing PABPC1-DDR2 were seeded on a 96-well glass-bottom plates and grown to 80-90% confluency. Inhibitor stock solutions in DMSO were added (final concentration 100 μM) to the media for 1 h, and fatty acid dye (Bodipy-C12, Red, 1 μM) was added 30 min prior to the experiment. Following appropriate starvation or control protocols, the cells were visualized by confocal microscopy and inclusion formation and fatty acid accumulation was assessed. Using this endogenously tagged PABPC1 cell line, the inhibitors were thus screened for the ability to induce lipid droplet formation, while also scoring stress-granule assembly (as depicted in FIG. 3A.).

Thirty-eight test molecules (inhibitors) induced PABPC1-positive inclusion formation (FIG. 3B). The majority of the molecules that we examined are known kinase inhibitors, so the high proportion of positive hits correlates with the known role of stress granules in cellular signaling, which involves multiple diverse protein kinases. The upregulation of LDs was estimated by calculating the accumulation of LD dye (fluorescent C12-Bodipy) in the samples that formed SGs and in the samples that did not result in SG accumulation (FIGS. 3B-3C). Despite variation, SG-forming cells significantly upregulated LDs, while the rest of the treated cells were not different form an untreated control (FIGS. 3B-3C). Thus, SG formation can be used as a predictor of LD biogenesis, and both SG formation and LD biogenesis can be used as markers of potential ALS-therapeutic treatments.

Variations of these readouts may include: 1. Simultaneous accumulation of SGs (observed as fluorescent puncta throughout the cell as opposed to diffuse fluorescent signal in normal/control conditions) and LDs (observed as large fluorescent puncta of fluorescent FAs in a chase experiment whereby fluorescent FAs are added to the media), 2. Increase in intracellular fluorescent FA signal (more than 50% above non-starved control), and 3. Accumulation of fluorescent FAs in puncta (50% more signal than control). These readouts can also be supported, augmented or substituted by measuring oxygen consumption rates and other direct metabolic readouts, using, for example, the Seahorse assay described below, in Example 4.

Table 1 lists the 136 small molecule inhibitors examined in these studies. Lipid effects are semi-quantitatively summarized, wherein “0” indicates no LD formation, “1” indicates traces of lipid present, “2” indicates widespread lipid and occasional lipid puncta (LD formation), and “3” indicates widespread lipid and frequent, highly localized lipid puncta or LD formation.

| Compound | Primary Target | Score |
|---|---|---|
| Control - No starvation | | 0 |
| Arsenite | Non-specific or multi-mechanism | 0 |
| | inhibitor of fatty acid oxidation | |
| Fasnall | inhibitor of fatty acid synthase (FASN) | 1 |
| PD 98059 | noncompetitive inhibitor of the MAPK | 1 |
| | pathway | |
| PD 169316 | selective inhibitor of p38 MAPK | 0 |
| TGX-221 | selective inhibitor of PI3K | 1 |
| (S)-H-1152 | potent, specific, ATP-competitive | 2 |
| | inhibitor of ROCK (Ki = 1.6 nM). | |
| AS-605240 | potent inhibitor of PI3-kinase γ | 1 |
| D-erythro-sphingosine | inhibits protein kinase C and | 3 |
| C18 | phosphatidic acid phosphohydrolase | |
| Lauric Acid Leelamide | Lauric acid leelamide is the lauric (C- | 0 |
| | 12) amide analog of leelamine. | |
| | Leelamine inhibits pyruvate | |
| | dehydrogenase kinase (PDHK) | |
| TG003 | Cdc2-like kinase (Clk) inhibitor | 0 |
| PKC 412 | reversible inhibitor of serine/threonine | 0 |
| | and tyrosine kinases, including | |
| | conventional PKC isoforms (α, β, and | |
| | γ), Syk, FLK1, Akt, PKA, c-Kit, C-Fgr, | |
| | c-Src, FLT3, PDFRβ, VEGFR1, and | |
| | VEGFR2 | |
| Doramapimod | a highly potent inhibitor of p38 MAPK | 0 |
| Paclitaxel | A potent mitotic inhibitor | 0 |
| Erlotinib | tyrosine kinase inhibitor which acts on | 0 |
| | the epidermal growth factor receptor | |
| | (EGFR), inhibiting EGFR-associated | |
| | kinase activity | |
| NVP-BEZ235 | a dual inhibitor of class I PI3Ks and | 0 |
| | mTOR | |
| Phthalazinone | potent, selective inhibitor of Aurora | 0 |
| pyrazole | kinase A | |
| AG-879 | A non-specific tyrphostin ErbB2 | 0 |
| | inhibitor | |
| 1-NA-PP1 | a reversible inhibitor of Src-family | 3 |
| | tyrosine kinases that have been mutated, | |
| | by a single base substitution, to become | |
| | ‘analog sensitive’ (as), as compared to | |
| | the wild-type kinase | |
| Torin 1 | a potent, selective inhibitor of mTOR | 0 |
| | (IC50 = 2 and 10 nM for mTORC1 and | |
| | mTORC2, respectively) | |
| BIBF 1120 | A VEGFR, FGFR, and PDGFR | 1 |
| | inhibitor | |
| SMI-4a | Pim kinase inhibitor | 3 |
| CAY 10657 | a thiophenecarboximide derivative | 1 |
| | proposed to inhibit IKK2 | |
| AS-703026 | selectively binds to and inhibits | 0 |
| | MEK1/2, | |
| Chelerythrine | a potent, cell permeable inhibitor of | 0 |
| | protein kinase C | |
| CAY 10578 | A potent and selective CK2 inhibitor | 0 |
| AZD 7762 | A selective checkpoint kinase inhibito | 1 |
| GSK 1059615 | A potent PI3K inhibitor | 1 |
| Ruxolitinib | A potent, selective JAK1/JAK2 | 1 |
| | inhibitor | |
| Necrostatin-1 | A RIP1 kinase inhibitor | 0 |
| SB-505124 | Inhibitor of receptors ALK4, ALK5, | 1 |
| | and ALK7 | |
| INK128 | Inhibitor of TORC1/2 | 0 |
| LY294002 | a selective phosphatidylinositol 3- | 0 |
| | kinase (PI3K) inhibitor | |
| SB-431542 | Inhibitor of receptors ALK4, ALK5, | 0 |
| | and ALK7 | |
| PD 184161 | A potent MEK1/2 inhibitor | 0 |
| Valproic Acid | class I HDAC inhibitor | 1 |
| PD 0325901 | MEK inhibitor | 0 |
| SB203580 | A specific p38 MAPK inhibitor | 0 |
| VX-702 | inhibitor of p38 MAP kinases | 0 |
| Emodin | Natural CK2 inhibitor and ER agonist | 0 |
| CHIR99021 | A selective GSK3 inhibitor | 0 |
| BIO | A potent, selective, and reversible | 0 |
| | GSK3 inhibitor | |
| Imatinib | An inhibitor of c-Abl, Bcr-Abl, | 0 |
| | PDGFR, and c-Kit | |
| Leelamine HCl | An inhibitor of pyruvate dehydrogenase | 2 |
| | kinase | |
| Arachidonic acid | A novel fatty acid amide | 0 |
| leelamide | | |
| PP2 | A selective inhibitor of Src tyrosine | 0 |
| | kinases | |
| 3-methyl-adenine | An inhibitor of autophagy | 0 |
| Y-27632 | a ROCK inhibitor | 0 |
| Bisinolyl maleimide | PKC & PKA | 0 |
| IV | | |
| Bisinolyl maleimide V | S6K | 0 |
| NSC 663284 | Inhibitor of Cdc25 isoforms | 0 |
| D 4476 | Inhibitor of CK1 and ALK5 | 0 |
| NU 7026 | Inhibitor of DNA-dependent protein | 0 |
| | kinase | |
| U-0126 | A MEK inhibitor and AMPK activator | 0 |
| H-9 | A potent, nonspecific kinase inhibitor | 0 |
| Indirubin-3-monoxime | GSK3β and cyclin-dependent kinases | 0 |
| NU 6102 | A potent Cdk1 and Cdk2 inhibitor | 0 |
| KN-62 | Ca2+/calmodulin-dependent kinase type | 0 |
| | II | |
| KN-93 | Ca2+/calmodulin-dependent kinase type | 3 |
| | II | |
| CGP 57380 | Inhibitor of MAPK-interacting kinase1 | 0 |
| Iso-olomoucine | An inactive stereoisomer of olomoucine | 0 |
| (S)-Glycyl-H-1152 | ROCK inhibitor | 0 |
| KN-92 | An inactive control compound for a | 0 |
| | CaMKII inhibitor | |
| Bisindolylmaleimide | PKCs & mixed other kinases | 0 |
| IX | | |
| ST638 | Tyrosine kinase inhibitor | 0 |
| Staurosporine | A potent inhibitor of protein kinase C | 0 |
| LY 364947 | Inhibitor of TGF-β type-1 receptors | 0 |
| SB 203580 | A specific p38 MAPK inhibitor | 0 |
| CAY 10621 | Selective inhibitor of SPHK1 | 0 |
| YM-201636 | Inhibitor of PIKfyve | 0 |
| PD 166326 | inhibitor of c-src and mixed receptor | 0 |
| | tyrosine kinases, including c-abl | |
| AS-041164 | Selective inhibitor of PI3Kγ | 0 |
| NVP-AEW541 | An IGF-IR antagonist | 0 |
| PP242 | Potent inhibitor of mTOR kinase in | 0 |
| | both mTORC1 and mTORC2 | |
| ABT-869 | A dual VEGFR and PDGFR family | 0 |
| | kinase inhibitor | |
| CAY 10622 | A ROCK1 and ROCK2 | 0 |
| 17b-hydroxy | PI3K | 0 |
| Wortmannin | | |
| O-1918 | antagonist of abnormal cannabidiol- | 0 |
| | mediated effects | |
| SU 6668 | mixed receptor tyrosine kinases | 0 |
| PHA-767491 | potent Cdc7 kinase inhibitor | 0 |
| AS-252424 | potent, selective for PI3-kinase γ | 3 |
| CAY 10505 | PI3Kγ | 2 |
| PI-103 | A potent PI3-kinase inhibitor | 2 |
| PIK-75 | Selective for PI3K p110α inhibitor | 3 |
| Sphingosine kinase | SPHK1 | 0 |
| inhibitor 2 | | |
| Piceatannol | potent resveratrol analog | 0 |
| SC-1 | promotes self-renewal of murine | 1 |
| | embryonic stem cells | |
| (R)-Roscovitine | potent for cyclin-dependent kinase 2 | 2 |
| BAY 43-9006 | a multi-kinase inhibitor | 0 |
| (Sorafenib) | | |
| CAY 10561 | selective for ERK | 3 |
| AS-604850 | selective for PI3Kγ | 3 |
| PI3 kinase alpha | A PI3K p110α | 2 |
| inhibitor 2 | | |
| CAY 10567 | Akt1 translocation inhibitor | 1 |
| ML-9 | PKB/Akt | 0 |
| Triciribine | potent inhibitor of Akt activation | 0 |
| Erbstatin analog | EGFR tyrosine kinase | 1 |
| Kenpaullone | of cyclin-dependent kinase and GSK3β | 1 |
| Olomoucine | cyclin-dependent kinases | 1 |
| AG-494 | EGF receptor kinase | 0 |
| AG-825 | Her2/Neu tyrosine kinase | 0 |
| AG-1478 | EGF receptor kinase | 0 |
| SB-216763 | GSK3a & b | 0 |
| SB-514286 | GSK3 | 0 |
| AG-17 | EGF receptor kinase | 2 |
| H-8 | nonspecific kinase inhibitor | 2 |
| LFM-A13 | BTK | 2 |
| SC-514 | IKK2 | 0 |
| Apigenin | CK2 | 0 |
| AG-18 | EGF receptor kinase | 3 |
| DRB | CTD kinase | 1 |
| RG-13022 | EGF receptor kinase | 0 |
| RG-14620 | EGF receptor kinase | 0 |
| AG-490 | JAK & mixed | 0 |
| AG-82 | EGF receptor kinase | 0 |
| AG-99 | EGF receptor kinase | 0 |
| AG-213 | EGF receptor kinase | 0 |
| AG-183 | EGF receptor kinase | 0 |
| Lavendustin C | EGF receptor kinase, Ca2+/calmodulin- | 1 |
| | dependent kinase type II and mixed | |
| | kinases | |
| ZM 336372 | Raf-1 | 0 |
| 5-Iodotubercidin | mixed kinases | 0 |
| SB 202190 | p38 MAP kinase | 0 |
| CAY 10571 | p38 & mixed mechanisms | 0 |
| Nilotinib | Bcr/Abl & mixed kinases | 0 |
| SP 600125 | pan-JNK | 0 |
| L-threo-sphingosine | a D-erythro-sphingosine analog | 0 |
| C18 | | |
| Myricetin | JAK1/STAT3 & MEK; inhibits TBARS | 0 |
| | formation | |
| HA-1077 | ROCK & mixed kinases | 0 |
| AG-370 | PDGF receptor kinase | 0 |
| Wortmannin | PI3K | 0 |
| AG-1296 | mixed protein kinases | 0 |
| Janex 1 (WHI-P131) | JAKs | 0 |
| CAY 10574 | Cdk2-cyclin E and Cdk9 inhibitor | 0 |
| CAY 10575 | IKK-ε | 0 |
| CAY 10576 | IKK-ε | 1 |
| NH125 | mammalian eEF-2K & mixed bacterial | 2 |
| | histidine kinases | |
| TWS119 | potent GSK3b inhibitor | 0 |
| NSC 210902 | Casein Kinase 2 (CK2) | 0 |
| CAY 10577 | Casein Kinase 2 (CK2) | 0 |

### Example 4: Evaluation of Exemplary Compounds on Fatty Acid Oxidation

The complementary assay for fatty acid trafficking will be how much cell are relying on the fatty acid oxidation in the mitochondria. For that a Seahorse XFe96 Flux Analyzer (Agilent) may be used with the commercially available MitoFuel Kit that is used to measure fatty acids metabolic dependency, for example, oxygen consumption rate. Cells will be seeded on a 96-well plate (Agilent), measurements were performed at a 70-90% confluency. Stress conditions that induce stress granule formation to recapitulate ALS-pathology, e.g. starvation, will be used.

### Example 5: Evaluating FAO Impairment in ALS-Patient Derived iPSCs

### Experimental Model and Subject Details

Patient-derived iPSCs (Cedar Sinai) were maintained in mTeSR1 (STEM Cell Technologies) media on Matrigel-coated plates. Differentiation to neural progenitor cells was carried out using STEMdiff neural induction protocol (STEM CELL technologies, with addition of SMADi). Neural progenitor cells were maintained in STEMdiff neural progenitor medium (STEM CELL Technologies).

Cells were grown to 70/6-90% confluency. After a PBS wash, the media was replaced with freshly prepared DMEM media without FBS, glucose, pyruvate, L-glutamine, and with 0.25 g/100 mL FA free bovine serum albumin. D-glucose (4 g/L), L-glutamine (2 mM), and synthetically defined lipid mixture 1 (1 μL/10 mL, Sigma) were added to the media in control conditions. Natural starvation was performed by incubating 90% confluent cells for 4 days without changing the medium.

Cells derived from an ALS patient with a mutation in TDP43 were compared with cells derived from a healthy control. First, induced pluripotent stem cells (iPSCs) were differentiated along a neuronal lineage in order to implement a starvation protocol without depriving cells of SC maintenance factors (FIGS. 2A-2D). Next, the time of starvation was optimized according to changes in induced fatty-acid oxidation, as it varies with the cell line and the type of starvation. TDP43 mutant cells failed to downregulate fatty-acid oxidation during long-term starvation compared with the healthy controls, similar to that observed in immortalized cell lines.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A method of modulating the oxidation of a fatty acid in a cell or subject exhibiting aberrant fatty acid oxidation, comprising administering to the cell or subject a mitochondrial lipid shunt modulator.

2. The method of claim 1, wherein the cell or subject exhibits aberrant fatty acid oxidation prior to administration of the mitochondrial lipid shunt modulator.

3. The method of claim 2, wherein the aberrant fatty acid oxidation comprises the prolongation of the elevation of fatty acid oxidation in response to starvation or metabolic stress.

4. The method of claim 1, wherein the aberrant fatty acid oxidation comprises an increase (e.g., at least 1, 2, 3, 4, 5, 7.5, 10, 15, 20% increase) in fatty acid oxidation, e.g., compared with a reference standard (e.g., a healthy cell).

5. The method of claim 4, wherein the increase comprises an increase in fatty acid oxidation for about 5, 10, 15, 30, 45 minutes, 1, 1.5, 2, 3, 4, 5, 6, 8, 10, 12, 16, 20, 24, 30, 36, or 48 hours, e.g., compared with a reference standard (e.g., a healthy cell).

6. The method of claim 1, wherein the aberrant fatty acid oxidation comprises an increase in cellular ATP levels (e.g., at least 1, 2, 3, 4, 5, 7.5, 10, 15, 20% increase), e.g., compared with a reference standard (e.g., a healthy cell).

7. The method of claim 1, wherein the aberrant fatty acid oxidation results in the accumulation of oxidative damage in the cell or subject (e.g., reactive oxygen species, acyl CoA, carbonylation of proteins).

8. The method of claim 1, wherein the modulating comprises reducing the oxidation of a fatty acid compared with a reference standard.

9. The method of claim 8, wherein the reducing comprises reducing the oxidation of a fatty acid by about 1, 2, 3, 4, 5, 7.5, 10, 15, 20%, e.g., compared to a reference standard.

10. The method of claim 1, wherein the oxidation comprises beta-oxidation.

11. The method of claim 1, wherein the oxidation is catalyzed by an enzyme, e.g., an acyl CoA dehydrogenase, e.g., hydroxyacyl-CoA dehydrogenase trifunctional multienzyme complex subunit alpha.

12. The method of claim 1, wherein the fatty acid comprises a medium chain, long chain fatty acid or a very long chain fatty acid.

13. The method of claim 1, wherein the fatty acid comprises linoleic acid, stearic acid, oleic acid, palmitic acid, linoelaidic acid, arachidonic acid, erucic acid, eicosapentaenoic acid, or docosahexaenoic acid.

14. The method of claim 1, wherein the cell or subject has a mutation in a gene.

15. The method of claim 14, wherein the gene is selected from a gene in the mitochondrial lipid shunt pathway.

16. The method of claim 14, wherein the gene is selected from TDP43, FUS OPTN, VCP, UBQLN1, CHCHD10, ALS2, ATX2, CCNF, C9orf72, SOD1, SETX, VAPB, SPG11, SQSTM1, PFN1, TBK1, and TIA1.

17. The method of claim 14, wherein the mutation is a mutation in the FUS gene.

18. The method of claim 17, wherein the FUS mutation is selected from R521G and R522G.

19. The method of claim 14, wherein the mutation is a mutation in the TDP43 gene.

20. The method of claim 19, wherein the TDP43 mutation is selected from Q331K, M337V, and G298S.

21. The method of claim 14, wherein the mutation is a mutation in the SOD1 gene.

22. The method of claim 21, wherein the SOD1 mutation is selected from G93A, G37R, and G85R.

23. The method of claim 1, wherein the cell or subject further comprises a decrease in fatty acid import into lipid droplets, e.g., compared with a reference standard.

24. The method of claim 23, wherein the cell or subject further comprises a decrease in lipid droplet size and/or lipid droplet number (e.g., about 1.5×, 2×, 3×, 4×, 5×, or 10×), e.g., compared with a reference standard.

25. The method of claim 1, wherein the mitochondrial lipid shunt modulator comprises a VDAC modulator, a CPT1 modulator, or an ACSL modulator.

26. The method of claim 1, wherein the mitochondrial lipid shunt modulator comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) e.g., as described herein.

27. The method of claim 1, wherein the mitochondrial lipid shunt modulator comprises a compound selected from (1)-(573) or a pharmaceutically acceptable salt or ester thereof, e.g., as described herein.

28. The method of claim 1, wherein the mitochondrial lipid shunt modulator is selected from erastin, erstin, efsevin, olesoxime, teglicar, perhexiline, amiodarone, 2-tetradecylglydate, and PA22, or an analog thereof.

29. The method of claim 1, wherein the mitochondrial lipid shunt modulator modulates the binding of VDAC2 to GSK3 or hexokinase.

30. The method of claim 1, wherein the modulating of GSK binding to VDAC2 results in modulating VDAC2 activity (e.g., phosphorylation of VDAC2).

31. The method of claim 1, wherein the mitochondrial lipid shunt modulator modulates the expression of VDAC or VDAC2.

32. The method of claim 1, wherein the mitochondrial lipid shunt modulator is an inhibitor of a protein kinase that regulates the activity of GSK3 binding to VDAC2, directly or through a regulatory pathway.

33. The method of claim 1, wherein the modulating of GSK3 binding to VDAC2 is achieved by modulating the activity of upstream kinases, e.g., kinases that deactivate or modulate GSK3 activity.

34. The method of claim 1, wherein the mitochondrial lipid shunt modulator binds to carnitine palmitoyltransferase I (CPTI).

35. The method of claim 1, wherein the mitochondrial lipid shunt modulator binds to long chain acyl CoA synthetase (ACSL).

36. The method of any one of claims 1-35, wherein the modulating of fatty acid oxidation comprises modulating the structural organization VDAC2 clustering in the mitochondrial membrane surface.

37. A method of modulating the formation of stress granules in a cell or subject exhibiting aberrant fatty acid oxidation, comprising administering to the cell or subject a mitochondrial lipid shunt modulator.

38. The method of claim 37, wherein the modulating comprises increasing the formation of stress granules (e.g., assisting formation of stress granules).

39. The method of any one of claims 37-38, wherein increasing stress granule formation results in modulating (e.g., decreasing) VDAC2 activity.

40. The method of claim 39, wherein increasing stress granule formation results in reduction of aberrant fatty acid oxidation.

41. The method of any one of claims 39-40, wherein the modulating comprises modulating the association of stress granules with the mitochondria and/or lipid droplets.

42. A method of modulating one or more of:
(i) the number of stress granules;
(ii) morphology of a stress granule, e.g., the size, shape, surface area to volume ratio;
(iii) the protein or nucleic acid composition of a stress granule;
(iv) the internal mobility or internal dynamics of a stress granule; and
(v) the phase of a stress granule;
in a cell or subject exhibiting aberrant fatty acid oxidation, comprising administering to the cell or subject a mitochondrial lipid shunt modulator.

43. The method of claim 42, comprising (i).

44. The method of any one of claims 42-43, comprising (ii).

45. The method of any one of claims 42-44, comprising (iii).

46. The method of any one of claims 42-45, comprising (iv).

47. The method of any one of claims 42-46, comprising (v).

48. A method of modulating the formation of lipid droplets in a cell or subject exhibiting aberrant fatty acid oxidation, comprising administering to the cell or subject a mitochondrial lipid shunt modulator.

49. The method of claim 48, wherein the modulating comprises reducing the formation of lipid droplets and/or preventing association of lipid droplets with the mitochondria or stress granules.

50. A method of modulating one or more of:
(i) the number of lipid droplets;
(ii) morphology of a lipid droplet, e.g., the size, shape, surface area to volume ratio;
(iii) the protein or nucleic acid composition of a lipid droplet;
(iv) the internal mobility or internal dynamics of a lipid droplet;
(v) the phase of a lipid droplet; and
(vi) the agglomeration state of a lipid droplet;
in a cell or subject exhibiting aberrant fatty acid oxidation, comprising administering to the cell or subject a mitochondrial lipid shunt modulator.

51. The method of claim 50, comprising (i).

52. The method of claim 50, comprising (ii).

53. The method of claim 50, comprising (iii).

54. The method of claim 50, comprising (iv).

55. The method of claim 50, comprising (v).

56. The method of claim 50, comprising (vi).

57. The method of claim 50, wherein modulating the formation and/or size or number of lipid droplets results in modulating VDAC2.

58. The method of claim 50, wherein the mitochondrial lipid shunt modulator comprises a VDAC modulator, a CPT1 modulator, or an ACSL modulator.

59. The method of claim 50, wherein the mitochondrial lipid shunt modulator comprises a compound of Formula (I), (II), (III), (IV), (V), (VI), (VII), (VIII), (IX), (X), (XI), (XII), (XIII), (XIV), (XV), (XVI), (XVII), (XIX), (XX), (XXI), (XXII), (XXIII), (XXIV), (XXV), (XXVI), (XXVII), (XXVIII), or (XXIX) or a pharmaceutically acceptable salt thereof, e.g., as described herein.

60. The method of claim 50, wherein the mitochondrial lipid shunt modulator comprises a compound selected from (1)-(573) or a pharmaceutically acceptable salt or ester thereof, e.g., as described herein.

61. The method of claim 50, wherein the mitochondrial lipid shunt modulator is selected from erastin, erstin, efsevin, olesoxime, teglicar, 2-tetradecylglydate, and PA22, or an analog thereof.

62. The method of claim 50, wherein the mitochondrial lipid shunt modulator modulates the binding of VDAC2 to GSK3 or hexokinase.

63. The method of claim 50, wherein the modulating of GSK binding to VDAC2 results in modulating VDAC2 activity (e.g., phosphorylation of VDAC2).

64. The method of claim 50, wherein the mitochondrial lipid shunt modulator modulates the expression of VDAC or VDAC2.

65. The method of claim 50, wherein the mitochondrial lipid shunt modulator is an inhibitor of a protein kinase that regulates the activity of GSK3 binding to VDAC2, directly or through a regulatory pathway.

66. The method of claim 50, wherein the modulating of GSK3 binding to VDAC2 is achieved by modulating the activity of upstream kinases, e.g., kinases that deactivate or modulate GSK3 activity.

67. The method of claim 50, wherein the mitochondrial lipid shunt modulator binds to carnitine palmitoyltransferase I (CPTI).

68. The method of any one of claims 42-67, wherein the mitochondrial lipid shunt modulator binds to long chain acyl CoA synthetase (ACSL).

69. The method of claim 50, wherein the modulating of fatty acid oxidation comprises modulating the structural organization VDAC2 clustering in the mitochondrial membrane surface.

70. A method of treating a neurodegenerative disease, e.g., amyotrophic lateral sclerosis, in a subject, wherein the subject (e.g., a cell in the subject) exhibits one or more of the following:
i) aberrant fatty acid oxidation;
ii) reduced fatty acid import into lipid droplets;
iii) reduced size and/or number of lipid droplets;
iv) decreased formation of stress granules;
v) formation of aberrant stress granules; and
vi) impaired VDAC2 function;
the method comprising administering to the subject a mitochondrial lipid shunt modulator, e.g., as described herein.

71. A method of treating a neurodegenerative disease, e.g., amyotrophic lateral sclerosis, in a subject, wherein the subject has or is identified as having a mutation in one or more of the following genes:
i) TDP-43;
ii) C9orf72; and
iii) FUS;
the method comprising administering to the subject a mitochondrial lipid shunt modulator, e.g., as described herein.

72. A method of evaluating a compound for the ability to act as a mitochondrial lipid shunt modulator, modulate fatty acid oxidation, regulate lipid droplet size or number, and/or regulate stress granule formation, comprising:
i) providing a library comprising a plurality of compounds;
ii) providing a plurality of cells, wherein one or more cells of the plurality is disposed in a reaction chamber;
iii) contacting a cell in the reaction chamber with a compound of the plurality of compounds; and
iv) evaluating a cell for modulation of fatty acid oxidation, oxidation, regulation of lipid droplet size or number, agglomeration state, and/or regulation of stress granule formation.

73. A method of evaluating the effect of a compound or plurality of compounds administered to a cell or human subject, for the ability to act as a mitochondrial lipid shunt modulator, modulate fatty acid oxidation, regulate lipid droplet size or number, and/or regulate stress granule formation, and the effects of which will be correlated with established a neurodegenerative disease (e.g., amyotrophic lateral sclerosis) or a pathophysiological disease marker, for use as:
i) a disease status diagnostic
ii) a disease progression diagnostic/biomarker
iii) a biomarker or diagnostic of therapeutic efficacy of administered compounds.
